(19) United States
(12) Patent Application Publication
(10) Pub. No.: US 2026/0347424 A1
(43) Pub. Date: Nov. 5, 2026

(54) INTERFERON PROPROTEINS AND USES THEREOF

(71) Applicant: Regeneron Pharmaceuticals, Inc., Tarrytown, NY (US)

(72) Inventors: Eva-Maria WEICK, New York, NY (US); Nicolin BLOCH, Elmsford, NY (US); Vidur GARG, White Plains, NY (US); Erica ULLMAN, Yorktown Heights, NY (US); Tong ZHANG, New Rochelle, NY (US); Chia-Yang LIN, Scarsdale, NY (US); Jiaxi WU, Pleasantville, NY (US); Eric SMITH, New York, NY (US)

(73) Assignee: Regeneron Pharmaceuticals, Inc., Tarrytown, NY (US)

(21) Appl. No.: 19/104,019

(22) PCT Filed: Aug. 18, 2023
(86) PCT No.: PCT/US2023/072512

§ 371 Date: Feb. 14, 2025

Related U.S. Application Data
(60) Provisional application No. 63/399,040, filed on Aug. 18, 2022.
(60) Provisional application No. 63/383,804, filed on Nov. 15, 2022.
(60) Provisional application No. 63/481,303, filed on Jan. 24, 2023.

(51) Int. Cl. C07K 14/555; A61K 38/00; C07K 16/28

(52) U.S. Cl. CPC C07K 14/555; C07K 16/2818; A61K 38/00; C07K 2317/522; C07K 2317/526; C07K 2317/55; C07K 2317/565; C07K 2319/30

(57) ABSTRACT

The present disclosure provides protease-activated IFN proproteins. The IFN proproteins comprises an IFN moiety that is sterically hindered from binding to its receptor through attachment to an Fc moiety via a protease-cleavable linker. Upon the action of a protease, e.g., at a tumor site, the protease-cleavable linker is cleaved, thereby releasing and activating the IFN moiety. IFN proproteins optionally further comprise a targeting moiety, e.g., a targeting moiety that recognizes a tumor-associated antigen and directs the proprotein to a tumor site. The disclosure further provides pharmaceutical compositions comprising the IFN proproteins, and methods of use of the IFN proproteins in therapy, as well as nucleic acids encoding the IFN proproteins, recombinant cells that express the IFN proproteins and methods of producing the IFN proproteins.

### 1. CROSS-REFERENCE TO RELATED APPLICATIONS

This application claims the priority benefit of U.S. provisional application No. 63/399,040, filed Aug. 18, 2022, U.S. provisional application No. 63/383,804, filed Nov. 15, 2022, and U.S. provisional application No. 63/481,303, filed Jan. 24, 2023, the contents of each of which are incorporated herein in their entireties by reference thereto.

### 2. SEQUENCE LISTING

The instant application contains a Sequence Listing which has been submitted electronically and is hereby incorporated by reference in its entirety. Said copy, created on Aug. 15, 2023, is named RGN-020WO_SL.xml and is 361, 113 bytes in size.

### 3. BACKGROUND

Type I interferons (IFNs) are thought to directly suppress tumor cell proliferation. Type I IFNs have utility in treatment of several types of cancer, including hematological tumors (chronic myeloid leukemia, hairy cell leukemia, multiple myeloma, and non-Hodgkin's lymphomas) and solid tumors (melanoma, renal carcinoma, and Kaposi's sarcoma). See, e.g., Zitvogel et al., 2015, Nat Rev Immunol 15:405-414 and Antonelli et al., 2015, Cytokine Growth Factor Rev 26:121-131.

A particular advantage of Type I IFN treatment is its ability to intervene at multiple points in the generation of anti-tumor immune responses, including stimulation of the innate and adaptive cytotoxic lymphocyte populations, negative regulation of suppressive cell types, its impact on tumor cells by inhibiting proliferation, and by modulating apoptosis, differentiation, migration, and cell surface antigen expression (Parker et al., 2016, Nature Reviews Cancer 16:131-144).

One of the biggest barriers to the use of Type I IFNs in the clinic is the severe side effects associated with such treatments. The most frequently encountered side effects are flu-like symptoms, hematological toxicity, elevated transaminases, nausea, fatigue, and psychiatric sequelae. These side effects hamper reaching and maintaining the doses needed for maximal therapeutic effect, and their occurrence can outweigh clinical benefit of Type I IFN treatment entirely (Lotrich, 2009, Dialogues Clin Neurosci 11:417-425). Type I IFNs signal through IFNAR1/IFNAR2 complex that are expressed on most cells and tissues in the body. Therefore, the ability to preferentially or specifically deliver active Type I IFNs to tumor-reactive immune cells (see, e.g., Diamond et al., 2011, J Exp Med. 208 (10): 1989-2003; Cauwels et al., 2018, Cancer Res. 78 (2): 463-474) or to the tumor microenvironment is imperative for continued clinical use of Type I IFN. Strategies are needed to modify Type I IFN in order to obtain new forms of the drug which preferentially exert their activity on tumor-reactive immune cells and/or at the tumor, and also to reduce side effects on normal IFNAR-expressing cells.

Thus, there is a need in the art for novel Type I IFN therapies with improved therapeutic efficacy and safety profiles.

### 4. SUMMARY

The present disclosure relates to IFN proproteins that are activated by proteases, e.g., proteases expressed in the tumor environment.

The IFN proproteins comprise an IFN moiety that is sterically hindered from binding to its receptor, configured so the IFN moiety is activated by cleavage of a linker in the IFN proprotein by a protease, resulting in alleviation of the steric hindrance of the IFN moiety. The IFN proproteins may further comprise a targeting moiety that directs the IFN proprotein to a particular tissue or cell type.

Typically, the IFN proproteins of the disclosure comprise two polypeptide chains, each comprising, from N- to C-terminus, a first linker, an interferon (IFN) moiety, a second linker, and an Fc domain. In some embodiments, the first linker and the second linker are both protease-cleavable linkers (PCLs). In other embodiments, only one of the first linker and the second linker is a PCL, with the other being a non-cleavable linker (NCL). Accordingly, in some embodiments, the first linker is a PCL, and the second linker is an NCL. In other embodiments, the first linker is an NCL, and the second linker is a PCL.

The IFN proproteins may further comprise, e.g., N-terminal to one or both Fc domains, a targeting moiety (or a component thereof, e.g., one chain of a Fab). The targeting moiety comprises an antigen-binding domain (“ABD”) that can, for example, bind to a target molecule present on the tumor surface (e.g., a tumor associated antigen) or other component in the tumor microenvironment (e.g., extracellular matrix (“ECM”) or tumor lymphocytes).

Exemplary IFN moieties that can be used in the IFN proproteins of the disclosure are described in Section 6.3.

Protease-cleavable linkers that can be used in the IFN proproteins of the disclosure are described in Section 6.4.

Non-cleavable linkers that can be used in the IFN proproteins of the disclosure are described in Section 6.5.

Targeting moieties that can be used in the IFN proproteins of the disclosure are described in Section 6.6 and targeting moiety formats are disclosed in Section 6.7.

Fc domains that can be incorporated into the IFN proproteins of the disclosure are described in Section 6.8.

Exemplary IFN proproteins of the disclosure are described in Section 6.2 and numbered embodiments 1 to 146.

The disclosure further provides nucleic acids encoding the IFN proproteins of the disclosure. The nucleic acids encoding the IFN proproteins can be a single nucleic acid (e.g., a vector encoding all polypeptide chains of an IFN proprotein) or a plurality of nucleic acids (e.g., two or more vectors encoding the different polypeptide chains of an IFN proprotein). The disclosure further provides host cells and cell lines engineered to express the nucleic acids and IFN proproteins of the disclosure. The disclosure further provides methods of producing an IFN proprotein of the disclosure. Exemplary nucleic acids, host cells, and cell lines, and methods of producing an IFN proprotein are described in Section 6.9 and numbered embodiments 147 to 149.

The disclosure further provides pharmaceutical compositions comprising the IFN proproteins of the disclosure. Exemplary pharmaceutical compositions are described in Section 6.10 and numbered embodiment 150.

Further provided herein are methods of using the IFN proproteins and the pharmaceutical compositions of the disclosure, e.g., for treating cancer. Exemplary methods are described in Section 6.11 and numbered embodiments 151 to 201.

### 5. BRIEF DESCRIPTION OF THE FIGURES

FIGS. 1A-1C are cartoons representing configurations of three IFN proproteins of the disclosure with protease-cleavable linkers flanking the interferon moiety. FIG. 1A represents an IFN proprotein having the overall configuration antibody-PCL-IFN-PCL-Fc; FIG. 1B represents a single hinge IFN proprotein having the configuration Fab-PCL-IFN-PCL-hinge-Fc; and FIG. 1C represents a double hinge IFN proprotein having the configuration Fab-hinge-PCL-IFN-PCL-hinge-Fc. Although shown as having targeting moieties in the forms of Fabs, the VH, VL and CL domains of the Fabs are optional for non-targeted IFN proproteins or could be replaced by other targeting moieties such as scFvs.

FIGS. 2A-2F are cartoons representing configurations of six IFN proproteins of the disclosure with protease-cleavable linkers (PCLs) only on one side of the interferon moiety and non-cleavable linkers (NCLs) on the other side of the interferon moiety. FIG. 2A represents an IFN proprotein having the overall configuration antibody-NCL-IFN-PCL-Fc; FIG. 2B represents a single hinge IFN proprotein having the configuration Fab-NCL-IFN-PCL-hinge-Fc; FIG. 2C represents a double hinge IFN proprotein having the configuration Fab-hinge-NCL-IFN-PCL-hinge-Fc; FIG. 2D represents an IFN proprotein having the overall configuration antibody-PCL-IFN-NCL-Fc; FIG. 2E represents a single hinge IFN proprotein having the configuration Fab-PCL-IFN-NCL-hinge-Fc; and FIG. 2F represents a double hinge IFN proprotein having the configuration Fab-hinge-PCL-IFN-NCL-hinge-Fc. Although shown as having targeting moieties in the forms of Fabs, the VH, VL and CL domains of the Fabs are optional for non-targeted IFN proproteins or could be replaced by other targeting moieties such as scFvs.

FIG. 3 is a table of exemplary targeted IFN proproteins according to FIG. 1 and their constituent polypeptide chains. TM refers to a targeting moiety; HC refers to an antibody heavy chain; LC refers to an antibody light chain; IFN refers to an interferon (IFN) moiety and ΔN and ΔC refers to N- and C-terminal truncations, respectively, in the IFN sequence of the IFN moiety (e.g., as described in Section 6.3); PCL refers to a protease-cleavable linker (e.g., as described in Section 6.4), Fc refers to an Fc domain (e.g., as described in Section 6.8); hinge refers to a hinge sequence of an antibody and LongHinge and ShortHinge refer to full length or truncated versions of an immunoglobulin hinge sequence (e.g., as described in Section 6.8.3). Further, although shown to have protease-cleavable linkers (PCLs) flanking the IFN moiety, one of the PCLs in each of Chain 1 and Chain 2 may be replaced by a non-cleavable linker (NCL), e.g., as described in Section 6.5.

FIGS. 4A-4C are the size exclusion ultra-performance liquid chromatography (SE-UPLC) profiles of exemplary IFN molecules that may be incorporated into the IFN proprotein constructs of the disclosure. FIG. 4A illustrates the SEC profile of an IFN molecule, Fc-IFNα1, with an N-terminal Fc domain and C-terminal IFN moiety; FIG. 4B illustrates the SEC profile of an IFN molecule, Fc-IFNα2b, with an N-terminal Fc and C-terminal IFN moiety; and FIG. 4C illustrates the SEC profile of an IFN molecule, IFNα2b-Fc, with an N-terminal IFN moiety and C-terminal Fc domain.

FIGS. 5A-5C depict the in vitro activity of exemplary IFN molecules that were linked to an Fc molecule either on the N- or the C-terminus. The cartoon images in FIG. 5A represent the N- and C-terminus Fc-fusions of IFN. Fc-IFN is the overall representation of IFN molecules with an N-terminal Fc domain and a C-terminal IFN moiety, whereas IFN-Fc is the overall representation of IFN molecules with an N-terminal IFN moiety and a C-terminal Fc domain. FIG. 5B is a graph showing the in vitro activity of exemplary IFN molecules, Fc-IFNα2b and IFNα2b-Fc, in comparison to unlinked IFNα2b. FIG. 5C is a graph showing the activity of Fc-IFN molecules in comparison to different unlinked IFNs.

FIGS. 6A-6D are the SE-UPLC profiles of exemplary mutant IFN molecules that may be incorporated into the IFN proprotein constructs of the disclosure. FIG. 6A illustrates the SE-UPLC profile of a mutant IFN molecule, Fc-IFNα2bR33A. FIG. 6B illustrates the SE-UPLC profile of a mutant IFN molecule, Fc-IFNα2bR149A. FIG. 6C illustrates the SE-UPLC profile of a mutant IFN molecule, Fc-IFNα2bR120A. FIG. 6D illustrates the SE-UPLC profile of a mutant IFN molecule, Fc-IFNα2bS152A.

FIGS. 7A-7B depict the in vitro activity of exemplary mutant IFN molecules that may be incorporated into the IFN proprotein constructs of the disclosure. The cartoon images in FIG. 7A represent the overall structure of wildtype (WT) or mutant (Mut) Fc-IFN molecules. FIG. 7B is a graph showing the in vitro activity of Fc-IFNα2b molecules with mutations affecting either the IFNAR1 or IFNAR2 interface.

FIGS. 8A-8C are the SE-UPLC profiles of exemplary IFN proprotein constructs. FIG. 8A illustrates the SE-UPLC profile of a single hinge full-length IFN proprotein, aPD1-singleHinge-FLIFN. FIG. 8B illustrates the SE-UPLC profile of a double hinge IFN proprotein, aPD1-doubleHingeLong-IFN. FIG. 8C illustrates the SEC profile of an IFN proprotein with two Fc regions.

FIGS. 9A-9B show the in vitro enzymatic cleavage of single and double hinge IFN proproteins. FIG. 9A is a gel image that illustrates the cleavage of three single hinge proproteins, two double hinge IFN proproteins, and a positive control by uPA. Similarly, FIG. 9B is a gel image that illustrates the cleavage of the same single and double IFN proproteins by MMP2 and MMP9.

FIGS. 10A-10B depict the in vitro activity of exemplary IFN proproteins in comparison to IFNα2b and Fc-IFNα2b. FIG. 10A is a graph showing the in vitro activity of two single hinge IFN proproteins (full length and truncated) and one double hinge IFN proprotein. FIG. 10B is a graph that illustrates changes in activity of the same IFN proproteins in FIG. 10A by the addition of MMP buffer alone (dashed line) or MMP buffer and enzyme mixture (dotted line).

### 6. DETAILED DESCRIPTION

### 6.1. Definitions

As used herein, the following terms are intended to have the following meanings:

ABD chain, targeting moiety chain: Targeting moieties and antigen binding sites (ABDs) within them can exist as one (e.g., in the case of an scFv or scFab) polypeptide chain or form through the association of more than one polypeptide chains (e.g., in the case of a Fab or an Fv). As used herein, the terms “ABD chain” and “targeting moiety chain” refer to all or a portion of an ABD or targeting moiety that exists on a single polypeptide chain. The use of the term “ABD chain” or “targeting moiety chain” is intended for convenience and descriptive purposes only and does not connote a particular configuration or method of production. Further, the reference to an ABD or targeting moiety when describing an IFN proprotein encompasses an ABD chain or targeting moiety chain unless the context dictates otherwise. Thus, when describing an IFN proprotein in which an Fc domain is operably linked to a targeting moiety, the Fc domain may be covalently linked directly or indirectly (e.g., via a linker) through a peptide bond to, e.g., (1) a first ABD or targeting moiety chain of a Fab or Fv (with the other components of the Fab or Fv on a second, associated ABD or targeting moiety chain) or (2) an ABD or targeting moiety chain containing an scFv or scFab.

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Activate, activation: The terms “activation”, “activation”, and the like in conjunction with an IFN proprotein of the disclosure refers to the protease-mediated enzymatic cleavage of a protease-cleavable linker that results in the release of an IFN moiety from a sterically-blocking constant domain.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding domain: The term “antigen-binding domain” or “ABD” as used herein refers to a portion of an antibody or antibody fragment (e.g., a targeting moiety) that has the ability to bind to an antigen non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F (ab) 2 fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23:1126-1136).

Associated: The term “associated” in the context of an IFN proprotein refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional IFN proprotein. Examples of associations that might be present in an IFN proprotein of the disclosure include (but are not limited to) associations between Fc domains to form an Fc region (homodimeric or heterodimeric as described in Section 6.8), associations between VH and VL regions in a Fab or Fv, and associations between CH1 and CL in a Fab.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, esophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), A1-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Constant domain: The terms “constant domain” refers to a CH1, CH2, CH3 or CL domain of an immunoglobulin.

The term “CH1 domain” refers to the heavy chain constant region linking the variable domain to the hinge in a heavy chain constant domain. In some embodiments, the term “CH1 domain” refers to the region of an immunoglobulin molecule spanning amino acids 118 to 215 (EU numbering). The term “CH1 domain” encompasses wildtype CH1 domains as well as variants thereof (e.g., non-naturally-occurring CH1 domains or modified CH1 domains). For example, the term “CH1 domain” includes wildtype IgG1, IgG2, IgG3 and IgG4 CH1 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH1 domains include CH1 domains with mutations that modify a biological activity of an antibody, such as ADCC, CDC or half-life.

The term “CH2 domain” refers to the heavy chain constant region linking the hinge to the CH3 domain in a heavy chain constant domain. In some embodiments, the term “CH2 domain” refers to the region of an immunoglobulin molecule spanning amino acids 238 to 340 (EU numbering). The term “CH2 domain” encompasses wildtype CH2 domains as well as variants thereof (e.g., non-naturally-occurring CH2 domains or modified CH2 domains). For example, the term “CH2 domain” includes wildtype IgG1, IgG2, IgG3 and IgG4 CH2 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH2 domains include CH2 domains with mutations that modify a biological activity of an antibody, such as ADCC, CDC, purification, dimerization and half-life.

The term “CH3 domain” refers to the heavy chain constant region that is C-terminal to the CH2 domain in a heavy chain constant domain. In some embodiments, the term “CH3 domain” refers to the region of an immunoglobulin molecule spanning amino acids 341 to 447 (EU numbering). The term “CH3 domain” encompasses wildtype CH3 domains as well as variants thereof (e.g., non-naturally-occurring CH3 domains or modified CH3 domains). For example, the term “CH3 domain” includes wildtype IgG1, IgG2, IgG3 and IgG4 CH3 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH3 domains include CH3 domains with mutations that modify a biological activity of an antibody, such as ADCC, CDC, purification, dimerization and half-life.

The term “CL domain” refers to the constant region of an immunoglobulin light chain. The term “CL domain” encompasses wildtype CL domains (e.g., kappa or lambda light chain constant regions) as well as variants thereof (e.g., non-naturally-occurring CL domains or modified CL domains). For example, the term “CL domain” includes wildtype kappa and lambda constant domains and variants thereof having 1, 2, 3, 4, 5, 1-3, 1-5, 3-5 and/or at most 5, 4, 3, 2, or 1 mutations, e.g., substitutions, deletions and/or additions.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case, it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. In some embodiments an Fc domain comprises a CH2 domain followed by a CH3 domain, with or without a hinge region N-terminal to the CH2 domain. The term “Fc region” refers to the region of formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Half Antibody: The term “half antibody” refers to a molecule that comprises at least one Fc domain and can associate with another molecule comprising an Fc through, e.g., a disulfide bridge or molecular interactions. A half antibody can be composed of one polypeptide chain or more than one polypeptide chains (e.g., the two polypeptide chains of a Fab). An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABD. Yet another example of a half antibody is a polypeptide comprising an scFv domain, a CH2 domain and a CH3 domain.

The IFN proproteins of the disclosure typically comprise two half antibodies, each comprising an IFN moiety flanked by protease-cleavable linkers, with an Fc domain C-terminal to the C-terminal protease-cleavable linker and a constant domain N-terminal to the N-terminal protease cleavable linker. One or both half antibodies in the IFN proproteins may further comprise a targeting moiety.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing a IFN proprotein of the disclosure, a host cell is preferably a cell line of mammalian origin or mammalian-like characteristics, such as monkey kidney cells (COS, e.g., COS-1, COS-7), HEK293), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., derivatives that grow at higher density than the original cell lines and/or glycan profile modified derivatives and and/or site-specific integration site derivatives.

Interferon: The term “interferon” as used herein refers to a full-length interferon or to a modified interferon, for example a truncated and/or mutant interferon. In some embodiments, the modified interferon is attenuated as compared to the corresponding wildtype interferon (e.g., retains less than 50%, less than 40%, less than 30%, less than 20%, or less than 10%, less than 1%, less than 0.1% or less than 0.05% activity in an in vitro luciferase reporter assay as described in Section 8.2.4). In some embodiments, the modified interferon is attenuated by a range bounded by any two of the foregoing values, e.g., 0.05%-50%, 0.1%-20%, 0.1%-10%, 0.05%-5%, 1%-20%, and so on and so forth. In other embodiments the modified interferon substantially retains the biological activity of the corresponding wildtype interferon (e.g., retains at least 50% activity in an in vitro luciferase reporter assay as described in Section 8.2.4). Interferons include Type I interferons (e.g., interferon-α and interferon-β) as well as Type II interferons (e.g., interferon-v).

Linker: The term “linker” as used herein refers to a protease-cleavable linker or a non-cleavable linker.

Non-cleavable linker: A non-cleavable linker as used herein refers to a peptide whose amino acid sequence lacks a substrate sequence for a protease, e.g., a protease as described in Section 6.4.1, that recognizes and cleaves a specific sequence motif, e.g., a substrate as described in Section 6.4.2.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a IFN proprotein of the disclosure, separate components (e.g., an Fc domain and an IFN moiety) can be operably linked directly or through peptide linker sequences. In the context of a nucleic acid encoding a fusion protein, such as a half antibody of an IFN proprotein of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Proprotein: A “proprotein” is a protein precursor that is inactive, and which can be activated by proteolysis by a protease. Thus, proproteins are “protease activatable”.

Protease: The term “protease” as used herein refers to any enzyme that catalyzes hydrolysis of a peptide bond. Generally, the proteases useful in the present disclosure, e.g., the proteases described in Section 6.4.1, recognize and cleaves a specific sequence motif, e.g., a substrate as described in Section 6.4.2. Preferably, the proteases are expressed at higher levels in cancer tissues as compared to normal tissues.

Protease-cleavable linker: As used herein, the term “protease-cleavable linker” or “PCL” refers to a peptide whose amino acid sequence contains one or more (e.g., two, three or more) substrate sequences for one or more proteases. Exemplary protease-cleavable linkers are described in Section 6.4 and exemplary protease-cleavable linker sequences are disclosed in Section 6.4.4.

Recognize: The term “recognize” as used herein refers to an antibody or antibody fragment (e.g., a targeting moiety) that finds and interacts (e.g., binds) with its epitope.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single-chain Fv” or “scFv” as used herein refers to ABDs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL and be arranged in the N- to C-terminal order VH-VL or VL-VH, typically separated by a linker, for example a linker as set forth in Table E.

Spacer: As used herein, the term “spacer” refers to a peptide, the amino acid sequence of which is not a substrate for a protease, incorporated into a linker containing a substrate. A spacer can be used to separate the substrate from other domains in a molecule, for example an ABD. In some aspects, residues in the spacer minimize aminopeptidase and/or exopeptidase action to prevent cleavage of N-terminal amino acids.

Specifically (or selectively) binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In preferred embodiments, the subject is human.

Substrate: The term “substrate” refers to peptide sequence on which a protease will act and within which the protease will cleave a peptide bond.

Target Molecule: The term “target molecule” as used herein refers to any biological molecule (e.g., protein, carbohydrate, lipid or combination thereof) expressed on a cell surface or in the extracellular matrix that can be specifically bound by a targeting moiety in an IFN proprotein of the disclosure.

Targeting Moiety: The term “targeting moiety” as used herein refers to any molecule or binding portion (e.g., an immunoglobulin or an antigen binding fragment) thereof that can bind to a cell surface or extracellular matrix molecule at a site to which an IFN proprotein of the disclosure is to be localized, for example on tumor cells or on lymphocytes in the tumor microenvironment. In some embodiments, the targeting moiety binds to a TAA. In other embodiments, the targeting moiety binds to a TCA. The targeting moiety can also have a functional activity in addition to localizing an IFN proprotein to a particular site. For example, a targeting moiety that binds to a checkpoint inhibitor such as PD1 can also exhibit anti-tumor activity or enhance the anti-tumor activity by IFN, for example by inhibiting PD1 signaling.

T-Cell Antigen, TCA: The term “T-cell antigen” or “TCA” refers to a molecule (typically a protein, carbohydrate, lipid or some combination thereof) that is expressed on the surface of a T-lymphocyte and is useful for the preferential targeting of a pharmacological agent to a particular site. In some embodiments, the site is cancer tissue and/or the T-cell antigen is a tumor reactive lymphocyte antigen, a cell surface molecule of tumor or viral lymphocytes, or a checkpoint inhibitor expressed on a T-lymphocyte.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen, TAA: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Treat, Treatment, Treating: As used herein, the terms “treat”, “treatment” and “treating” refer to the reduction or amelioration of the progression, severity and/or duration of a disorder, or the amelioration of one or more symptoms (preferably, one or more discernible symptoms) of a disorder resulting from the administration of one or more IFN receptor agonists (e.g., IFN proproteins that are capable of agonizing an IFN receptor, for example following activation) of the disclosure. In some embodiments, the disorder is a proliferative disorder and the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Universal Light Chain, UCL: The term “universal light chain” or “ULC” as used herein refers to a light chain variable region (VL) that can pair with more than on heavy chain variable region (VL). In the context of a targeting moiety, the term “universal light chain” or “ULC” refers to a light chain polypeptide capable of pairing with the heavy chain region of the targeting moiety and also capable of pairing with other heavy chain regions. ULCs can also include constant domains, e.g., a CL domain of an antibody. Universal light chains are also known as “common light chains.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. IFN Proproteins

The present disclosure relates to IFN proproteins comprising an IFN moiety that is attenuated by steric hindrance from flanking constant domains. The IFN proprotein is configured such that upon encountering a protease, e.g., a protease that is overexpressed in the tumor environment, the protease-cleavable linker is cleaved and IFN is released. This is achieved by incorporating an IFN moiety between constant/Fc domains of an antibody, flanked by protease-cleavable linkers (PCL). Accordingly, these proproteins are sometimes referred to as “internal” IFN constructs. IFN proproteins capable of agonizing an IFN receptor, for example following activation, are sometimes referred to herein as “IFN receptor agonists.”

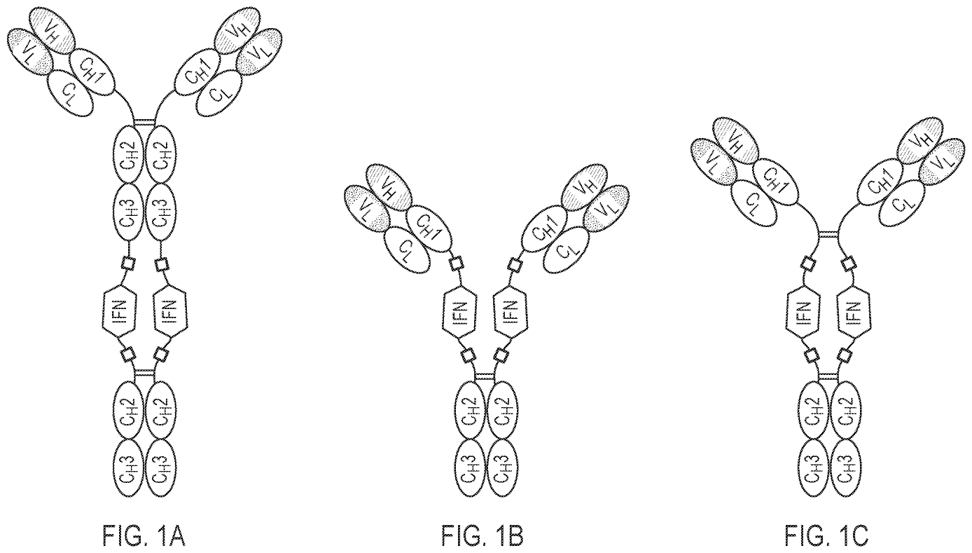

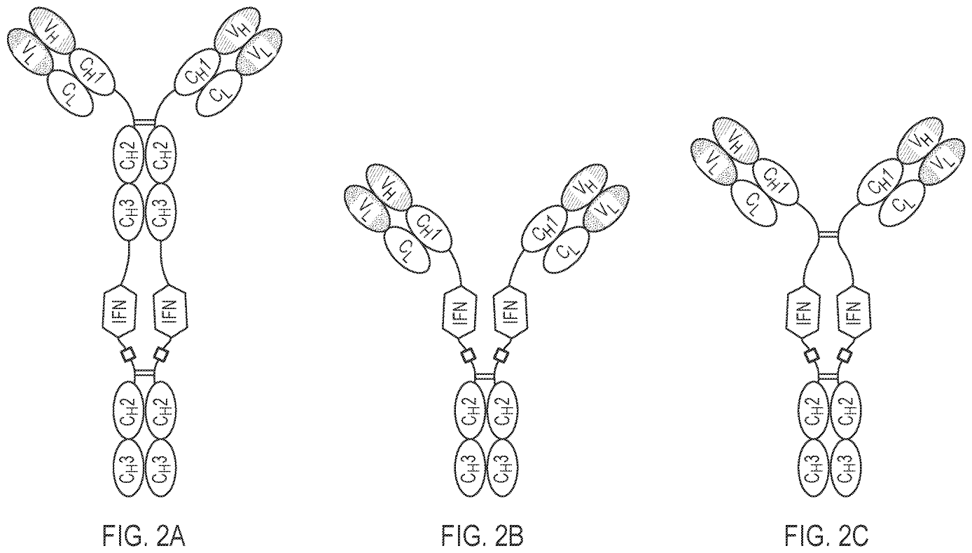

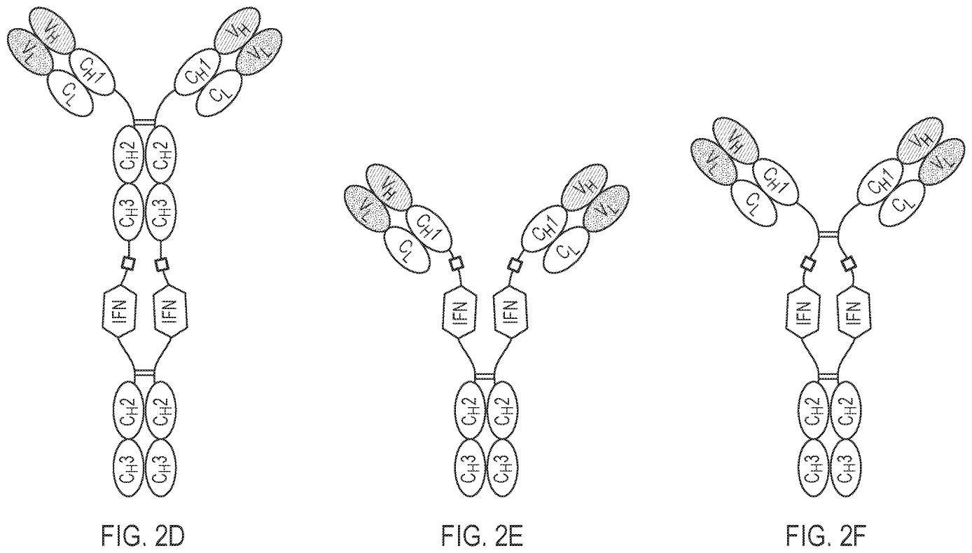

Generally, the IFN proproteins are composed of two half antibodies, comprising a pair of Fc domains that associate to form an Fc region (typically comprising hinge sequences), N-terminal to which are linkers which may be cleavable or non-cleavable, the IFN moieties, and additional linkers which may be cleavable or non-cleavable. In some embodiments, each half antibody comprises two protease-cleavable linkers, flanking the IFN moiety. Exemplary IFN proproteins with two protease-cleavable linkers in each half antibody are illustrated in FIGS. 1A-1C. In other embodiments, each half antibody comprises a single protease-cleavable linker, on one side of the IFN moiety, with the linker on the other side of the IFN moiety being a non-cleavable linker. Exemplary IFN proproteins with one protease-cleavable linker in each half antibody are illustrated in FIGS. 2A-2F. Further to the N-terminal of the protease-cleavable linkers are antibody constant domains, either entire constant domains comprising Fc domains, which associate to form another Fc region, or only portions of constant domains (e.g., CH1 domains).

Exemplary configurations of IFN proproteins of the disclosure are depicted in FIGS. 1A-1C and FIGS. 2A-2F. As illustrated, the IFN moiety and the surrounding linkers may comprise hinge domains at only their C-termini (“single hinge,” e.g., as illustrated in FIG. 1A, FIGS. 1B, 2A, 2B, 2D and 2E) or both their N- and C-termini (“double hinge”, e.g., as illustrated in FIGS. 1C, 2C and 2F).

Generally, the IFN proprotein comprises:
a) a first polypeptide chain comprising:
   i) a first immunoglobulin constant domain;
   ii) a first linker;
   iii) a first Type I interferon (IFN) moiety;
   iv) a second linker and
   v) a first Fc domain; and
b) a second polypeptide chain comprising:
   i) a second immunoglobulin constant domain;
   ii) a third linker;
   iii) a second Type I interferon (IFN) moiety;
   iv) a fourth linker; and
   v) a second Fc domain associated with the first Fc domain to form an Fc region;

In some embodiments, the first, second, third and fourth linkers are all protease-cleavable linkers. In other embodiments, only two of the first, second, third and fourth linkers are protease-cleavable linkers. For example, in certain embodiments, the first and third linkers are protease-cleavable linkers and the second and four linkers are non-cleavable linkers. In other embodiments, the first and third linkers are non-cleavable linkers and the second and four linkers are protease-cleavable linkers.

Preferably, the IFN moieties in the IFN proprotein are sterically hindered from binding to an IFN receptor by the Fc domains and/or constant domains.

An exemplary IFN proprotein is depicted in FIG. 1A, where the targeting moiety (depicted as a Fab domain represented by a VH-CH1 paired with a VL-CL) is optional. The IFN proprotein of FIG. 1A comprises a first polypeptide chain, a second polypeptide chain, an optional third polypeptide chain and an optional fourth polypeptide chain, wherein:
a) the first polypeptide chain comprises:
   i) an optional first VH1 domain;
   ii) a first CH1 domain;
   iii) a first Fc domain comprising a hinge domain, a CH2 domain and a CH3 domain, wherein the CH3 domain is the first immunoglobulin constant domain;
   iv) a first protease cleavable linker (PCL);
   v) a first Type I interferon (IFN) moiety;
   vi) a second protease cleavable linker (PCL); and
   vii) a second Fc domain;
b) the second polypeptide chain comprises;
   i) an optional second VH1 domain;
   ii) a second CH1 domain;
   iii) a third Fc domain comprising a hinge domain, a CH2 domain and a CH3 domain, wherein the CH3 domain is the second immunoglobulin constant domain;
   iv) a third protease cleavable linker (PCL);
   v) a second Type I interferon (IFN) moiety;
   vi) a fourth protease cleavable linker (PCL); and
   vii) a fourth Fc domain;
c) the optional third polypeptide chain comprises:
   i) a first VL domain;
   ii) a first CL domain; and
d) the fourth polypeptide chain comprises:
   i) a second VL domain; and
   ii) a second CL domain;
      wherein the first polypeptide chain is associated with the second polypeptide chain such that the first Fc domain and the third Fc domain associate to form an Fc region and the second Fc domain and the fourth Fc domain form another Fc region.

When present, the third polypeptide chain associates with the first polypeptide chain such that the first VH, CH1, VL and CL form a first targeting moiety and the fourth polypeptide chain associates with the second polypeptide chain polypeptide such that the second VH, CH1, VL and CL form the second targeting moiety. Alternatively, the first and second polypeptide chains may include scFvs at their N-termini.

Thus, the first polypeptide chain (together with the third polypeptide chain, if present) represents a first half antibody and the second polypeptide chain (together with the fourth polypeptide chain, if present) represents a second half antibody.

Another exemplary IFN proprotein is depicted in FIG. 1B, where the targeting moiety (depicted as a Fab domain represented by a VH-CH1 paired with a VL-CL) is optional. The IFN proprotein of FIG. 1B comprises a first polypeptide chain, a second polypeptide chain, an optional third polypeptide chain and an optional fourth polypeptide chain, wherein:
a) the first polypeptide chain comprises:
   i) an optional first VH1 domain;
   ii) a first CH1 domain;
   iii) a first protease cleavable linker (PCL);
   iv) a first Type I interferon (IFN) moiety;
   v) a second protease cleavable linker (PCL); and
   vi) a first Fc domain;
b) the second polypeptide chain comprises;
   i) an optional second VH1 domain;
   ii) a second CH1 domain;
   iii) a third protease cleavable linker (PCL);
   iv) a second Type I interferon (IFN) moiety;
   v) a fourth protease cleavable linker (PCL); and
   vi) a second Fc domain;
c) the optional third polypeptide chain comprises:
   i) a first VL domain;
   ii) a first CL domain; and
d) the fourth polypeptide chain comprises:
   i) a second VL domain; and
   ii) a second CL domain;
      wherein the first polypeptide chain is associated with the second polypeptide chain such that the first Fc domain and the second Fc domain form an Fc region.

When present, the third polypeptide chain associates with the first polypeptide chain such that the first VH, CH1, VL and CL form a first targeting moiety and the fourth polypeptide chain associates with the second polypeptide chain polypeptide such that the second VH, CH1, VL and CL form the second targeting moiety. Alternatively, the first and second polypeptide chains may include scFvs at their N-termini.

Thus, the first polypeptide chain (together with the third polypeptide chain, if present) represents a first half antibody and the second polypeptide chain (together with the fourth polypeptide chain, if present) represents a second half antibody.

A variant of the IFN proprotein of FIG. 1B is depicted in FIG. 1C. The IFN proprotein of FIG. 1C further includes a first hinge domain between the first CH1 domain and the first protease-cleavable linker and a second hinge domain between the second CH1 domain and the third protease cleavable linker.

Another exemplary IFN proprotein is depicted in FIG. 2A, where the targeting moiety (depicted as a Fab domain represented by a VH-CH1 paired with a VL-CL) is optional. The IFN proprotein of FIG. 2A comprises a first polypeptide chain, a second polypeptide chain, an optional third polypeptide chain and an optional fourth polypeptide chain, wherein:
a) the first polypeptide chain comprises:
   i) an optional first VH1 domain;
   ii) a first CH1 domain;
   iii) a first Fc domain comprising a hinge domain, a CH2 domain and a CH3 domain, wherein the CH3 domain is the first immunoglobulin constant domain;
   iv) a first non-cleavable linker (NCL);
   v) a first Type I interferon (IFN) moiety;
   vi) a first protease cleavable linker (PCL); and
   vii) a second Fc domain;
b) the second polypeptide chain comprises;
   i) an optional second VH1 domain;
   ii) a second CH1 domain;
   iii) a third Fc domain comprising a hinge domain, a CH2 domain and a CH3 domain, wherein the CH3 domain is the second immunoglobulin constant domain;
   iv) a second non-cleavable linker (NCL);
   v) a second Type I interferon (IFN) moiety;
   vi) a second protease-cleavable linker (PCL); and
   vii) a fourth Fc domain;
c) the optional third polypeptide chain comprises:
   i) a first VL domain;
   ii) a first CL domain; and
d) the fourth polypeptide chain comprises:
   i) a second VL domain; and
   ii) a second CL domain;
      wherein the first polypeptide chain is associated with the second polypeptide chain such that the first Fc domain and the third Fc domain associate to form an Fc region and the second Fc domain and the fourth Fc domain form another Fc region.

When present, the third polypeptide chain associates with the first polypeptide chain such that the first VH, CH1, VL and CL form a first targeting moiety and the fourth polypeptide chain associates with the second polypeptide chain polypeptide such that the second VH, CH1, VL and CL form the second targeting moiety. Alternatively, the first and second polypeptide chains may include scFvs at their N-termini.

Thus, the first polypeptide chain (together with the third polypeptide chain, if present) represents a first half antibody and the second polypeptide chain (together with the fourth polypeptide chain, if present) represents a second half antibody.

Another exemplary IFN proprotein is depicted in FIG. 2B, where the targeting moiety (depicted as a Fab domain represented by a VH-CH1 paired with a VL-CL) is optional. The IFN proprotein of FIG. 2B comprises a first polypeptide chain, a second polypeptide chain, an optional third polypeptide chain and an optional fourth polypeptide chain, wherein:
a) the first polypeptide chain comprises:
   i) an optional first VH1 domain;
   ii) a first CH1 domain;
   iii) a first non-cleavable linker (NCL);
   iv) a first Type I interferon (IFN) moiety;
   v) a first protease-cleavable linker (PCL); and
   vi) a first Fc domain;
b) the second polypeptide chain comprises;
   i) an optional second VH1 domain;
   ii) a second CH1 domain;
   iii) a second non-cleavable linker (NCL);
   iv) a second Type I interferon (IFN) moiety;
   v) a second protease-cleavable linker (PCL); and
   vi) a second Fc domain;
c) the optional third polypeptide chain comprises:
   i) a first VL domain;
   ii) a first CL domain; and
d) the fourth polypeptide chain comprises:
   i) a second VL domain; and
   ii) a second CL domain;
      wherein the first polypeptide chain is associated with the second polypeptide chain such that the first Fc domain and the second Fc domain form an Fc region.

When present, the third polypeptide chain associates with the first polypeptide chain such that the first VH, CH1, VL and CL form a first targeting moiety and the fourth polypeptide chain associates with the second polypeptide chain polypeptide such that the second VH, CH1, VL and CL form the second targeting moiety. Alternatively, the first and second polypeptide chains may include scFvs at their N-termini.

Thus, the first polypeptide chain (together with the third polypeptide chain, if present) represents a first half antibody and the second polypeptide chain (together with the fourth polypeptide chain, if present) represents a second half antibody.

A variant of the IFN proprotein of FIG. 2B is depicted in FIG. 2C. The IFN proprotein of FIG. 2C further includes a first hinge domain between the first CH1 domain and the first non-cleavable linker and a second hinge domain between the second CH1 domain and the second non-cleavable linker.

Another exemplary IFN proprotein is depicted in FIG. 2D, where the targeting moiety (depicted as a Fab domain represented by a VH-CH1 paired with a VL-CL) is optional. The IFN proprotein of FIG. 2D comprises a first polypeptide chain, a second polypeptide chain, an optional third polypeptide chain and an optional fourth polypeptide chain, wherein:
a) the first polypeptide chain comprises:
   i) an optional first VH1 domain;
   ii) a first CH1 domain;
   iii) a first Fc domain comprising a hinge domain, a CH2 domain and a CH3 domain, wherein the CH3 domain is the first immunoglobulin constant domain;
   iv) a first protease-cleavable linker (PCL);
   v) a first Type I interferon (IFN) moiety;
   vi) a first non-cleavable linker (NCL); and
   vii) a second Fc domain;
b) the second polypeptide chain comprises;
   i) an optional second VH1 domain;
   ii) a second CH1 domain;
   iii) a third Fc domain comprising a hinge domain, a CH2 domain and a CH3 domain, wherein the CH3 domain is the second immunoglobulin constant domain;
   iv) a second protease cleavable linker (PCL);
   v) a second Type I interferon (IFN) moiety;
   vi) a second non-cleavable linker (NCL); and
   vii) a fourth Fc domain;
c) the optional third polypeptide chain comprises:
   i) a first VL domain;
   ii) a first CL domain; and
d) the fourth polypeptide chain comprises:
   i) a second VL domain; and
   ii) a second CL domain;
      wherein the first polypeptide chain is associated with the second polypeptide chain such that the first Fc domain and the third Fc domain associate to form an Fc region and the second Fc domain and the fourth Fc domain form another Fc region.

When present, the third polypeptide chain associates with the first polypeptide chain such that the first VH, CH1, VL and CL form a first targeting moiety and the fourth polypeptide chain associates with the second polypeptide chain polypeptide such that the second VH, CH1, VL and CL form the second targeting moiety. Alternatively, the first and second polypeptide chains may include scFvs at their N-termini.

Thus, the first polypeptide chain (together with the third polypeptide chain, if present) represents a first half antibody and the second polypeptide chain (together with the fourth polypeptide chain, if present) represents a second half antibody.

Another exemplary IFN proprotein is depicted in FIG. 2E, where the targeting moiety (depicted as a Fab domain represented by a VH-CH1 paired with a VL-CL) is optional. The IFN proprotein of FIG. 2E comprises a first polypeptide chain, a second polypeptide chain, an optional third polypeptide chain and an optional fourth polypeptide chain, wherein:
a) the first polypeptide chain comprises:
   i) an optional first VH1 domain;
   ii) a first CH1 domain;
   iii) a first protease cleavable linker (PCL);
   iv) a first Type I interferon (IFN) moiety;
   v) a first non-cleavable linker (NCL); and
   vi) a first Fc domain;
b) the second polypeptide chain comprises;
   i) an optional second VH1 domain;
   ii) a second CH1 domain;
   iii) a second protease cleavable linker (PCL);
   iv) a second Type I interferon (IFN) moiety;
   v) a second non-cleavable linker (NCL); and
   vi) a second Fc domain;
c) the optional third polypeptide chain comprises:
   i) a first VL domain;
   ii) a first CL domain; and
d) the fourth polypeptide chain comprises:
   i) a second VL domain; and
   ii) a second CL domain;
      wherein the first polypeptide chain is associated with the second polypeptide chain such that the first Fc domain and the second Fc domain form an Fc region.

When present, the third polypeptide chain associates with the first polypeptide chain such that the first VH, CH1, VL and CL form a first targeting moiety and the fourth polypeptide chain associates with the second polypeptide chain such that the second VH, CH1, VL and CL form the second targeting moiety. Alternatively, the first and second polypeptide chains may include scFvs at their N-termini.

Thus, the first polypeptide chain (together with the third polypeptide chain, if present) represents a first half antibody and the second polypeptide chain (together with the fourth polypeptide chain, if present) represents a second half antibody.

A variant of the IFN proprotein of FIG. 2E is depicted in FIG. 2F. The IFN proprotein of FIG. 2E further includes a first hinge domain between the first CH1 domain and the first protease-cleavable linker and a second hinge domain between the second CH1 domain and the second protease cleavable linker.

Typically, the Fc domains in the IFN proproteins depicted in FIGS. 1A-1C and FIGS. 2A-2F include hinge domains.

Accordingly, the IFN proproteins typically include two to four protease-cleavable linkers. Cleavage of all protease-cleavable linkers in IFN proproteins with four protease-cleavable linkers results in release of an activated IFN protein comprising the IFN moiety and lacking the C-terminal Fc moiety, the N-terminal constant domain, and, if present, a targeting moiety. Cleavage of all protease-cleavable linkers in IFN proproteins with two protease-cleavable linkers results in removal of one of the sterically blocking portions and yields an IFN molecule comprising a targeting moiety (antibody-IFN) or an Fc domain (IFN-Fc).

In some embodiments, this configuration is advantageously utilized for IFN proproteins comprising a targeting moiety that binds to a TAA or ECM target molecule that is expressed in the tumor environment. Without intending to be bound by theory, the inventors believe that in this configuration, the targeting moiety targets the IFN proprotein to the tumor environment, where proteases cleave the protease-cleavable linkers resulting in the release of an IFN protein comprising the IFN moiety and linker sequences. This locally activated IFN protein then induces an immune response against the cancer cells.

Sequence and length of hinge and linker sequences can be varied, as can the sequence of the IFN moiety (containing either the full-length or N- and/or C-terminal truncated IFN sequence). Exemplary IFN moieties are described in Section 6.3 and include IFNα- and IFNβ-based moieties as described in Sections 6.3.1 and 6.3.2 below. Exemplary protease cleavable linker sequences as disclosed in Section 6.4. Exemplary non-cleavable linker and hinge sequences are disclosed in Sections 6.5 and 6.8.3, respectively. Exemplary targeting moieties are disclosed in Section 6.6.

### 6.3. The IFN Moiety

There are two major classes of IFNs: Type I (IFN-α subtypes, IFN-β, etc.) and Type II (IFN-γ). Additional IFNs (IFN-like cytokines; IFN-λ subtype) have also been identified.

The IFN moiety of the disclosure may comprise any wild type or modified (e.g., truncated and/or mutant) IFN or IFN-like cytokine sequence but preferably is a Type I IFN moiety. Type I IFNs bind a heterodimeric plasma membrane receptor IFNAR made of IFNAR1 and IFNAR2 that is ubiquitously expressed in all nucleated cells. Ligand binding is initiated by high-affinity receptor subunit IFNAR2 (Piehler et al., 2012, Immunological Reviews, doi.org/10.1111/imr. 12001). As such, Type I IFNs are able to act on virtually all cells of the body. Sixteen Type I interferon subtypes have been identified, which vary in their intrinsic variability in affinity to IFNAR2 and activity.

In some embodiments, the Type I IFN moiety is an interferon-α (IFNα) moiety. In other embodiments, Type I IFN moiety is an interferon-β (IFNβ) moiety.

In other embodiments, the Type I IFN moiety is an interferon-ω (IFNω), interferon-ε (IFNε) or interferon-κ (IFNκ) moiety.

The Type I IFN moiety may comprise a sequence that varies from a wild-type IFN sequence by one or more mutations, e.g., substitutions, deletions, or insertions. Substitutions that attenuate IFN activity by reducing receptor binding may suitably be used. Amino acids with N- or C-terminal deletions (or truncations) may also be used, e.g., a truncation of 1, 2, 3, 4, 5, 6, 7, 8, 9, 10, 11, 12, 13, 14 or 15 amino acids from the N- and/or C-termini of a mature Type I IFN. Without being bound by theory, the present inventors believe that the terminal truncations impose additional steric constraints on the IFN moiety and reduce IFN activity until cleavage of a protease-cleavable linker in the IFN proproteins.

Further details of exemplary Type I IFN moieties are provided below.

### 6.3.1. Interferon-α Moieties

The IFNα gene is a member of the alpha interferon gene cluster on chromosome 9. The encoded cytokine is a member of the Type I interferon family that is produced in response to viral infection as a key part of the innate immune response with potent antiviral, antiproliferative and immunomodulatory properties. IFNα refers to a family of proteins, with at least 15 known subtypes of human IFNα. The major subtypes identified are IFNα1, IFNα2, IFNα8, IFNα10, IFNα14 and IFNα21.

The IFNα1 gene has two allelic variants: IFNα1a and IFNα1b. The amino acid sequence of human IFNα1a is assigned UniProtKB accession number P01562, reproduced below with the signal peptide is underlined:

(SEQ ID NO: 1)
MASPFALLMV LVVLSCKSSC SLGCDLPETH SLDNRRTLML
LAQMSRISPS SCLMDRHDFG FPQEEFDGNQ FQKAPAISVL
HELIQQIFNL FTTKDSSAAW DEDLLDKFCT ELYQQLNDLE
ACVMQEERVG ETPLMNADSI LAVKKYFRRI TLYLTEKKYS
PCAWEVVRAE IMRSLSLSTN LQERLRRKE

The human IFNα1b gene differs the IFNα1a allelic variant by one base change in the coding region, leading to a single change in amino acid sequence (Val114 instead of Ala114 in the mature protein, corresponding to Val137 instead of Ala137 in the full-length polypeptide).

There are three allelic variants of IFNα2 alleles, IFNα2a, IFNα2b and IFNα2c. Allele IFNα2b is the predominant allele while allele IFNα2a is less predominant and IFNα2c only a minor allelic variant. The amino acid sequence of human IFNα2 is assigned UniProtKB accession number P01563. The sequence of the IFNα2b allele is reproduced below with the signal peptide is underlined:

(SEQ ID NO: 2)
MALTFALLVA LLVLSCKSSC SVGCDLPQTH SLGSRRTLML
LAQMRRISLF SCLKDRHDFG FPQEEFGNQF QKAETIPVLH
EMIQQIFNLF STKDSSAAWD ETLLDKFYTE LYQQLNDLEA
CVIQGVGVTE TPLMKEDSIL AVRKYFQRIT LYLKEKKYSP
CAWEVVRAEI MRSFSLSTNL QESLRSKE

IFNα2b has an arginine (R) at position 23 of the mature protein while IFNα2a has a lysine (K). Thus, in some embodiments, the IFNα2 moiety has an arginine at the position corresponding to position 23 of the mature protein. In other embodiments, the IFNα2 moiety has a lysine at the position corresponding to position 23 of the mature protein.

In various aspects, the IFNα moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFNα1a, IFNα1b, and/or IFNα2b, IFNα2a, or IFNα2c or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of mature IFNα1a, IFNα1b, and/or IFNα2b, IFNα2a, or IFNα2c).

In some embodiments, the IFNα moiety has one or more amino acid substitutions, e.g., substitutions that alter IFNAR binding and/or agonism. Exemplary substitutions are found in WO 2013/107791, U.S. Pat. No. 8,258,263, WO2007/000769A2, WO2008/124086, WO2010/030671, WO2018/144999A1, and WO2015/007520, WO 2013/059885, WO2020156467A1, WO2021/126929A1. In some embodiments, the IFNα moiety comprises:
a) one or more substitutions selected from L15A, A19W, R22A, R23A, L26A, F27A, L30A, L30V, K31A, D32A, R33K or R33A or R33Q, H34A, D35A, Q40A, H57Y, E58N, Q61S, F64A, N65A, T69A, L80A, D82E, Y85A, T861, Y89A, D114R or D114A, L117A, R120A or R120E or R120K, K121E, R125A, K133A, K134A, R144A, A145G or A145M, M148A, R149A, R149K, S152A, L153A, N156A; and/or
b) one or more substitutions at amino acids 57-89 and 159-165 described in WO2007000769A2; and/or
c) one or more amino acid substitutions at 9, 17, 47, 65, 66, 117, 123, 128, 147, and 157 to alanine, glycine, or threonine described in WO2021126929A1.

The amino acid positions of the foregoing substitutions are given with reference to mature IFNα2b.

In further embodiments, the IFNα moiety comprises one or more amino acid substitutions set forth in Table 1. Table 1 sets forth IFNα substitutions identified by reference to the amino acid position within the sequence of IFNα2.

TABLE 1
Exemplary IFNα mutations (with reference to the sequence of mature IFNα2)

| IFNα sequence mutation(s) | Source/impact of substitution(s) |
|---|---|
| L15A | WO2018014068A9 |
| R22A | WO2018014068A9 |
| R23A | WO2018014068A9 |
| S25A | WO2018014068A9 |
| L26A | Thomas et al., 2011, Cell, 146(4): 621-632 |
| F27A | Thomas et al., 2011, Cell, 146(4): 621-632 |
| L30A | Thomas et al., 2011, Cell, 146(4): 621-632 |
| L30V | WO2018014068A9 |
| K31A | WO2018014068A9 |
| D32A | WO2018014068A9 |
| R33K | WO2018014068A9 |
| R33Q | WO2018014068A9 |
| R33A | WO2013059885A2 |
| H34A | WO2018014068A9 |
| D35E | WO2016201337A1 |
| Q40A | WO2018014068A9 |
| H57A | Thomas et al., 2011, Cell, 146(4): 621-632 |
| H57S | WO2013134138A1 |
| E58A | Thomas et al., 2011, Cell, 146(4): 621-632 |
| Q61A | WO2016201337A1 |
| Q90A | Piehler et al., 2012, Immunological Reviews, |
| | doi.org/10.1111/imr.12001 |
| E96A | Piehler et al., 2012, Immunological Reviews, |
| | doi.org/10.1111/imr.12001 |
| D114R | WO2018014068A9 |
| L117A | WO2018014068A9 |
| R120A | WO2018014068A9 |
| R120E | WO2018014068A9 |
| Q124R | WO2022015711A1 |
| R125A | WO2018014068A9 |
| R125E | WO2018014068A9 |
| K131A | WO2018014068A9 |
| E132A | WO2018014068A9 |
| K133A | WO2018014068A9 |
| K134A | WO2018014068A9 |
| L135A | WO2013134138A1 |
| R144A | WO2022015711A1 |
| R144D | WO2018014068A9 |
| R144E | WO2018014068A9 |
| R144G | WO2018014068A9 |
| R144H | WO2018014068A9 |
| R144I | WO2018014068A9 |
| R144K | WO2018014068A9 |
| R144L | WO2013059885A2 |
| R144N | WO2018014068A9 |
| R144Q | WO2018014068A9 |
| R144S | WO2013059885A2 |
| R144T | WO2013059885A2 |
| R144V | WO2018014068A9 |
| R144Y | WO2013059885A2 |
| A145D | WO2013059885A2 |
| A145E | WO2018014068A9 |
| A145G | WO2018014068A9, WO2022015711A1 |
| A145H | WO2013059885A2 |
| A145I | WO2018014068A9 |
| A145K | WO2013059885A2 |
| A145L | WO2018014068A9 |
| A145M | WO2018014068A9 |
| A145N | WO2018014068A9 |
| A145Q | WO2018014068A9 |
| A145S | WO2018014068A9 |
| A145T | WO2018014068A9 |
| A145V | WO2018014068A9 |
| A145Y | WO2013059885A2 |
| M148A | WO2018014068A9 |
| R149A | WO2022015711A1, Piehler et al., 2012, |
| | Immunological Reviews, doi.org/10.1111/imr.12001 |
| R149K | Piehler et al., 2012, Immunological Reviews, |
| | doi.org/10.1111/imr.12001 |
| S152A | Piehler et al., 2012, Immunological Reviews, |
| | doi.org/10.1111/imr.12001, WO2022015711A1 |
| L153A | WO2018014068A9 |
| N156A | WO2018014068A9 |
| R162A | WO2016201337A1 |
| E165D | WO2016201337A1 |
| L30A, H57Y, E58N, Q61S | WO2018014068A9 |
| R33A, H57Y, E58N, Q61S | WO2013059885A2 |
| H57S, E58S, Q61S | Alter IFNα-IFNAR1 binding affinity at Site 1 |
| H57Y, E58N, Q61S | WO2007000769A2 |
| N65A, L80A, Y85A, Y89A | WO2018014068A9 |
| N65A, L80A, Y85A, Y89A, | WO2018014068A9 |
| D114A | |
| N65A, L80A, Y85A, Y89A, | WO2018014068A9 |
| L174A | |
| N65A, L80A, Y85A, Y89A, | WO2018014068A9 |
| R120A | |
| Y85A, Y89A, D114A | WO2018014068A9 |
| Q90A, R120A | Piehler et al., 2012, Immunological Reviews, |
| | doi.org/10.1111/imr.12001 |
| D114A, R120A | WO2018014068A9 |
| L117A, R120A | WO2018014068A9 |
| L117A, R120A, K121A | WO2018014068A9 |
| R120A, K121A | WO2018014068A9 |
| R120E, K121E | WO2018014068A9 |
| R144A, H57Y, E58N, Q61S | WO2013059885A2 |
| M148A, H57Y, E58N, Q61S | WO2018014068A9 |
| R149A, R162A | WO2013134138A1 |
| L153A, H57Y, E58N, Q61S | WO2018014068A9 |
| Deletion of residues L161 to | WO2018014068A9 |
| E165 | |

In some embodiments, the IFNα moiety comprises an amino acid sequence comprising the amino acid substitution R33A or R33K, Q90A, E96A, R120A, A145M, R149A or R149K, S152A, or any combination of two or more of the foregoing, e.g., Q90A+R120A or A145M+R149K.

The sequences of exemplary IFNα moieties that can be utilized in the IFN proproteins of the disclosure are set forth in Table 2 below:

TABLE 2
Exemplary IFNα Moieties

| | | SEQ ID |
|---|---|---|
| Construct | Sequence | NO: |
| IFNα1a | CDLPETHSLDNRRTLMLLAQMSRISPSSCLMDRHDFGFPQEEFDG | |
| | NQFQKAPAISVLHELIQQIFNLFTTKDSSAAWDEDLLDKFCTELYQQ | |
| | LNDLEACVMQEERVGETPLMNADSILAVKKYFRRITLYLTEKKYSP | |
| | CAWEVVRAEIMRSLSLSTNLQERLRRKE | |
| IFNα1b | CDLPETHSLDNRRTLMLLAQMSRISPSSCLMDRHDFGFPQEEFDG | 3 |
| | NQFQKAPAISVLHELIQQIFNLFTTKDSSAAWDEDLLDKFCTELYQQ | |
| | LNDLEACVMQEERVGETPLMNVDSILAVKKYFRRITLYLTEKKYSP | |
| | CAWEVVRAEIMRSLSLSTNLQERLRRKE | |
| IFNα2a | CDLPQTHSLGSRRTLMLLAQMRKISLFSCLKDRHDFGFPQEEFGN | 4 |
| | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMRSFSLSTNLQESLRSKE | |
| IFNα2b | CDLPQTHSLGSRRTLMLLAQMRRISLFSCLKDRHDFGFPQEEFGN | 5 |
| | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEWVRAEIMRSFSLSTNLQESLRSKE | |
| IFNα2b | CDLPQTHSLGSRRTLMLLAQMRRISLFSCLKDAHDFGFPQEEFGN | 6 |
| (R33A) | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMRSFSLSTNLQESLRSKE | |
| IFNα2b | CDLPQTHSLGSRRTLMLLAQMRRISLFSCLKDKHDFGFPQEEFGN | 7 |
| (R33K) | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMRSFSLSTNLQESLRSKE | |
| IFNα2b | CDLPQTHSLGSRRTLMLLAQMRRISLFSCLKDRHDFGFPQEEFGN | 8 |
| (Q90A) | QFQKAETIPVLHEMIQQIFNLFSTKDSSAAWDETLLDKFYTELYAQL | |
| | NDLEACVIQGVGVTETPLMKEDSILAVRKYFQRITLYLKEKKYSPCA | |
| | WEVVRAEIMRSFSLSTNLQESLRSKE | |
| IFNα2b | CDLPQTHSLGSRRTLMLLAQMRRISLFSCLKDRHDFGFPQEEFGN | 9 |
| (E96A) | QFQKAETIPVLHEMIQQIFNLFSTKDSSAAWDETLLDKFYTELYQQL | |
| | NDLAACVIQGVGVTETPLMKEDSILAVRKYFQRITLYLKEKKYSPCA | |
| | WEVVRAEIMRSFSLSTNLQESLRSKE | |
| IFNα2b | CDLPQTHSLGSRRTLMLLAQMRRISLFSCLKDRHDFGFPQEEFGN | 10 |
| (R120A) | QFQKAETIPVLHEMIQQIFNLFSTKDSSAAWDETLLDKFYTELYQQL | |
| | NDLEACVIQGVGVTETPLMKEDSILAVAKYFQRITLYLKEKKYSPCA | |
| | WEVVRAEIMRSFSLSTNLQESLRSKE | |
| IFNα2b | CDLPQTHSLGSRRTLMLLAQMRRISLFSCLKDRHDFGFPQEEFGN | 11 |
| (A145M) | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRMEIMRSFSLSTNLQESLRSKE | |
| IFNα2b | CDLPQTHSLGSRRTLMLLAQMRRISLFSCLKDRHDFGFPQEEFGN | 12 |
| (R149A) | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMASFSLSTNLQESLRSKE | |
| IFNα2b | CDLPQTHSLGSRRTLMLLAQMRRISLFSCLKDRHDFGFPQEEFGN | 13 |
| (R149K) | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMKSFSLSTNLQESLRSKE | |
| IFNα2b | CDLPQTHSLGSRRTLMLLAQMRRISLFSCLKDRHDFGFPQEEFGN | 14 |
| (S152A) | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMRSFALSTNLQESLRSKE | |
| ΔN-IFN-ΔC | THSLGSRRTLMLLAQMRRISLFSCLKDRHDFGFPQEEFGNQFQKA | 15 |
| | ETIPVLHEMIQQIFNLFSTKDSSAAWDETLLDKFYTELYQQLNDLEA | |
| | CVIQGVGVTETPLMKEDSILAVRKYFQRITLYLKEKKYSPCAWEVV | |
| | RAEIMRSFSLSTNLQ | |
| IFN-ΔC | CDLPQTHSLGSRRTLMLLAQMRRISLFSCLKDRHDFGFPQEEFGN | 16 |
| | QFQKAETIPVLHEMIQQIFNLFSTKDSSAAWDETLLDKFYTELYQQL | |
| | NDLEACVIQGVGVTETPLMKEDSILAVRKYFQRITLYLKEKKYSPCA | |
| | WEVVRAEIMRSFSLSTNLQ | |

### 6.3.2. Interferon 1-β Moieties

Interferon 1-β (IFN1β or IFN 1-β) is a cytokine that is naturally produced by the immune system in response to biological and chemical stimuli. IFN1β is a glycosylate, secreted monomer having a molecular weight of around 22 kDa that is produced in large quantities by fibroblasts and as such it is also known as fibroblast interferon. IFN1β binds to the IFNAR receptor composed of the IFNAR1 and IFNAR2 dimers to induce signaling via the JAK/STAT pathway and other pathways. IFN1β can also function by binding to IFNAR1 alone and signal independently of the Jak-STAT pathways (see, e.g., Stanifer et al., 2019, Int. J Mol. Sci. 20 (6): 1445).

IFN1β contains 5 α-helices designated A (YNLLGFLQRSSNFQCQKLL (SEQ ID NO: 18)), B (KEDAALTIYEMLQNIFAIF (SEQ ID NO: 19)), C (ETIVENLLANVYHQINHLKTVLEEKL (SEQ ID NO: 20)), D (SSLHLKRYYGRILHYLKA (SEQ ID NO: 21)), and E (HCAWTIVRVEILRNFYFINRLT (SEQ ID NO: 22)). The five α-helices are interconnected by loops of 2-28 residues designated AB, BC, CD and DE loops. It has been reported that the A helix in the AB loop and the E helix in the DE loop are involved in the binding of IFN1β to the IFNAR receptor.

Two types of IFN1β have been described: Interferon 1-β1 (IFN1β1) and Interferon 1-3 (IFN1β3).

The amino acid sequence of human IFNβ precursor is listed under GenBank: accession number AAA36040.1 and reproduced below (with the signal peptide underlined):

(SEQ ID NO: 23)
MTNKCLLQIA LLLCFSTTAL SMSYNLLGFL QRSSNFQCQK
LLWQLNGRLE YCLKDRMNFD IPEEIKQLQQ FQKEDAALTI
YEMLQNIFAI FRQDSSSTGW NETIVENLLA NVYHQINHLK
TVLEEKLEKE DFTRGKLMSS LHLKRYYGRI LHYLKAKEYS
HCAWTIVRVE ILRNFYFINR LTGYLRN

In various aspects, the IFNβ moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFN1β1 or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFN1β1).

In various embodiments, the IFNβ moiety comprises one or more amino acid substitutions and/or deletions as compared to IFN1β1. In some embodiments, the substitution is a C17S (with reference to the mature IFN1β1) and the deletions are one of the C-terminal truncations described in US 2009/0025106 A1 as IFN-ΔI, IFNA2, IFNA3, IFNA4, IFNA5, IFNA6, IFN-Δ7, IFN-Δδ, IFNA9, and IFN-ΔI O.

### 6.3.3. Other Type I Interferons

In certain aspects, the Type I IFN moiety is other than an IFNα or IFNβ moiety, e.g., an interferon-ω (IFNω), interferon-ε (IFNε) or interferon-κ (IFNκ) moiety.

Human IFNω is identified by UniProt accession no. P05000 and the IFNω1 allele has the amino acid sequence set forth below, with the signal sequence underlined:

(SEQ ID NO: 24)
MALLFPLLAALVMTSYSPVGSLGCDLPQNHGLLSRNTLVLLHQMRRISPF
LCLKDRRDFRFPQEMVKGSQLQKAHVMSVLHEMLQQIFSLFHTERSSAAW
NMTLLDQLHTGLHQQLQHLETCLLQVVGEGESAGAISSPALTLRRYFQGI
RVYLKEKKYSDCAWEVVRMEIMKSLFLSTNMQERLRSKDRDLGSS

In various aspects, the IFNω moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFN1ω1 or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFN1001).

Human IFNε is identified by UniProt accession no. Q86WN2 and has the amino acid sequence set forth below, with the signal sequence underlined:

(SEQ ID NO: 25)
MIIKHFFGTVLVLLASTTIFSLDLKLIIFQQRQVNQESLKLLNKLQTLSI
QQCLPHRKNFLLPQKSLSPQQYQKGHTLAILHEMLQQIFSLFRANISLDG
WEENHTEKFLIQLHQQLEYLEALMGLEAEKLSGTLGSDNLRLQVKMYFRR
IHDYLENQDYSTCAWAIVQVEISRCLFFVFSLTEKLSKQGRPLNDMKQEL
TTEFRSPR

In various aspects, the IFNε moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFN1ε or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFN1ε).

Human IFNκ is identified by UniProt accession no. Q9P0W0 and has the amino acid sequence set forth below, with the signal sequence underlined:

(SEQ ID NO: 26)
MSTKPDMIQKCLWLEILMGIFIAGTLSLDCNLLNVHLRRVTWQNLRHLSSM
SNSFPVECLRENIAFELPQEFLQYTQPMKRDIKKAFYEMSLQAFNIFSQHT
FKYWKERHLKQIQIGLDQQAEYLNQCLEEDKNENEDMKEMKENEMKPSEAR
VPQLSSLELRRYFHRIDNFLKEKKYSDCAWEIVRVEIRRCLYYFYKFTALF
RRK

In various aspects, the IFNκ moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFN1κ or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFN1κ).

### 6.4. Protease-Cleavable Linkers

The IFN proproteins of the disclosure typically comprise four linkers, referred to in the numbered embodiments below as the first, second, third and fourth linkers, with the first and second linkers on one polypeptide chain and the third and fourth linkers on another polypeptide chain. Two to four of the linkers are protease-cleavable linkers. In some embodiments, the first, second, third and fourth linkers are all protease-cleavable linkers. In other embodiments, only two of the first, second, third and fourth linkers are protease-cleavable linkers. For example, in certain embodiments, the first and third linkers are protease-cleavable linkers and the second and fourth linkers are non-cleavable linkers. In other embodiments, the first and third linkers are non-cleavable linkers and the second and fourth linkers are protease-cleavable linkers.

A protease-cleavable linker can range from 8 amino acids to 100 or more amino acids. In various embodiments, the protease-cleavable linker ranges from 8 amino acids to 15 amino acids, from 10 amino acids to 20 amino acids, 20 amino acids to 80, and in certain aspects a non-cleavable peptide linker ranges from 20 amino acids to 60 amino acids, 20 amino acids to 40 amino acids, from 30 amino acids to 50 amino acids, from 20 amino acids to 80 amino acids, or from 30 amino acids to 70 amino acids in length.

The protease-cleavable linkers comprise one or more substrate sequences for one or more proteases, for example one or more of the proteases set forth in Section 6.4.1. The one or more substrate sequences, e.g., one or more of the substrate sequences set forth in Section 6.4.2, are typically (but not necessarily) flanked by one or more spacer sequences, e.g., spacer sequences as described in Section 6.4.3. Each protease-cleavable linker can include one, two, three or more substrate sequences. The spacer sequences can be adjoining, overlapping, or separated by spacer sequences. Preferably, the C- and N-termini of the protease-cleavable linkers contain spacer sequences.

In various aspects of IFN proproteins comprising four protease-cleavable linkers, the first and third protease-cleavable linkers are cleavable by the same protease and/or the second and fourth protease-cleavable linkers are cleavable by the same protease. In some embodiments, the protease is a protease set forth in Table A.

In further aspects of IFN proproteins comprising four protease-cleavable linkers, the first and third protease-cleavable linkers comprise the same substrate sequence(s) and/or the second and fourth protease-cleavable linkers comprise the same substrate sequence(s). In some embodiments, the substrate sequence(s) are set forth in Table B. In further embodiments, the first and third protease-cleavable linkers also comprise the same spacer sequence(s) and/or the second and fourth protease-cleavable linkers also comprise the same spacer sequence(s). In some embodiments, the spacer sequence(s) are set forth in Table C.

In further aspects IFN proproteins comprising four protease-cleavable linkers, the first and third linkers comprise the same linker sequence(s) and/or the second and fourth linkers comprise the same linker sequence(s). In some embodiments, the linker sequence(s) are set forth in Table D.

In some embodiments of IFN proproteins comprising four protease-cleavable linkers, the first and third protease-cleavable linkers are the same as the second and fourth protease-cleavable linkers.

In other embodiments, the first and third protease-cleavable linkers are different from the second and fourth protease-cleavable linkers.

In the foregoing aspects and embodiments, the different linkers may be cleavable by the same protease, different proteases, or when a linker comprises multiple substrate sequences, the different linkers may be cleavable by multiple proteases, one or more of which are common and one or more of which are different.

Exemplary protease-cleavable linker sequences are set forth in Section 6.4.4

### 6.4.1. Proteases

Exemplary protease whose substrate sequences can be incorporated into the protease-cleavable linkers are set forth in Table A below.

TABLE A
Exemplary Proteases for Substrate Cleavage

| | | |
|---|---|---|
| ADAMS, ADAMTS, e.g. | Caspases, e.g., | MMP24 |
| ADAM8 | Caspase 1 | MMP26 |
| ADAM9 | Caspase 2 | MMP27 |
| ADAM10 | Caspase 3 | |
| ADAM12 | Caspase 4 | |
| ADAM15 | Caspase 5 | |
| ADAM17/TACE | Caspase 6 | |
| ADAMDEC1 | Caspase 7 | |
| ADAMTS1 | Caspase 8 | Cysteine proteinases, e.g., |
| ADAMTS4 | Caspase 9 | Cruzipain |
| ADAMTS5 | Caspase 10 | Legumain |
| | Caspase 14 | Otubain-2 |
| Aspartate proteases, e.g., | | |
| BACE | Cysteine cathepsins, e.g., | KLKs, e.g., |
| Renin | Cathepsin B | KLK4 |
| | Cathepsin C | KLK5 |
| Aspartic cathepsins, e.g., | Cathepsin K | KLK6 |
| Cathepsin D | Cathepsin L | KLK7 |
| Cathepsin E | Cathepsin S | KLK8 |
| | Cathepsin V/L2 | KLK10 |
| NS3/4A | Cathepsin X/Z/P | KLK11 |
| PACE4 | | KLK13 |
| Plasmin | MMPs, e.g., | KLK14 |
| PSA | MMP1 | |
| tPA | MMP2 | Metallo proteinases, e.g., |
| Thrombin | MMP3 | Meprin |
| Tryptase | | Neprilysin |
| uPA | MMP7 | PSMA |
| | MMP8 | BMP-1 |
| Type II Transmembrane | MMP9 | |
| Serine Proteases (TTSPs), | MMP10 | |
| e.g., | | |
| DESC1 | MMP11 | Serine proteases, e.g., |
| DPP-4 | MMP12 | activated protein C |
| FAP | MMP13 | Cathepsin A |
| Hepsin | MMP14 | Cathepsin G |
| Matriptase-2 | MMP15 | Chymase |
| MT/SP1/Matriptase | MMP16 | coagulation factor proteases |
| | MMP17 | (e.g., FVIIa, FIXa, FXa, |
| | | FXIa, FXIIa) |
| TMPRSS2 | MMP19 | Human Neutrophil Elastase |
| TMPRSS3 | MMP20 | Lactoferrin |
| TMPRSS4 | MMP23 | |

In particular embodiments, the protease is matrix metalloprotease (MMP)-2, MMP-9, legumain asparaginyl endopeptidase, thrombin, fibroblast activation protease (FAP), MMP-1, MMP-3, MMP-7, MMP-8, MMP-12, MMP-13, MMP-14, membrane type 1 matrix metalloprotease (MT1-MMP), plasmin, transmembrane protease, serine (TMPRSS-3/4), cathepsin A, cathepsin B, cathepsin D, cathepsin E, cathepsin F, cathepsin H, cathepsin K, cathepsin L, cathepsin L2, cathepsin O, cathepsin S, caspase 1, caspase 2, caspase 3, caspase 4, caspase 5, caspase 6, caspase 7, caspase 8, caspase 9, caspase 10, caspase 11, caspase 12, caspase 13, caspase 14, human neutrophil elastase, urokinase/urokinase-type plasminogen activator (uPA), a disintegrin and metalloprotease (ADAM) 10, ADAM12, ADAM17, ADAM with thrombospondin motifs (ADAMTS), ADAMTS5, beta secretase (BACE), granzyme A, granzyme B, guanidinobenzoatase, hepsin, matriptase, matriptase 2, meprin, neprilysin, prostate-specific membrane antigen (PSMA), tumor necrosis factor-converting enzyme (TACE), kallikrein-related peptidase (KLK) 3, KLK5, KLK7, KLK11, NS3/4 protease of hepatitis C virus (HCV-NS3/4), tissue plasminogen activator (tPA), calpain, calpain 2, glutamate carboxypeptidase II, plasma kallikrein, AMSH-like protease, AMSH, γ-secretase component, antiplasmin cleaving enzyme (APCE), decysin 1, apoptosis-related cysteine peptidase, or N-acetylated alpha-linked acidic dipeptidase-like 1.

### 6.4.2. Substrates

Exemplary substrate sequences that are cleavable by a tumor protease and can be incorporated into the protease-cleavable linkers are set forth in Table B below.

TABLE B
Substrate Sequences for Protease-Cleavable Linkers

| | | Cleaving | SEQ ID |
|---|---|---|---|
| Substrate Sequence | Designation | Protease | NO: |
| (DE)₈RPLALWRS(DR)₈ | SU1 | MMP7 | 27 |
| AARGPAIH | SU2 | | 28 |
| AAYHLVSQ | SU3 | Collagenase | 29 |
| AGLGISST | SU4 | Collagenase | 30 |
| AGLGVVER | SU5 | Collagenase | 31 |
| ALAL | SU6 | Lysosomal | 32 |
| | | Enzyme | |
| ALFFSSPP | SU7 | | 33 |
| ALFKSSFP | SU8 | | 34 |
| ALLLALL | SU9 | TOP | 35 |
| AQFVLTEG | SU10 | Collagenase | 36 |
| AQNLLGMV | SU11 | | 37 |
| AVGLLAPP | SU12 | Serine protease | 38 |
| DAFK | SU13 | Urokinase | 39 |
| | | plasminogen | |
| | | activator (uPA) | |
| DEVD | SU14 | Caspase-3 | 40 |
| DEVDP | SU15 | Caspase-3 | 41 |
| DPRSFL | SU16 | Thrombin | 42 |
| DVAQFVLT | SU17 | Collagenase | 43 |
| DVLK | SU18 | Plasmin | 44 |
| DWLYWPGI | SU19 | | 45 |
| EDDDDKA | SU20 | Enterokinase | 46 |
| EP(Cit)G(Hof)YL | SU21 | MMP2, MMP9, | 47 |
| | | MMP14 | |
| EPQALAMS | SU22 | Collagenase | 48 |
| ESLPVVAV | SU23 | Collagenase | 49 |
| ESPAYYTA | SU24 | MMP | 50 |
| F(Pip)RS | SU25 | Thrombin | |
| FK | SU26 | Lysosomal | |
| | | Enzyme | |
| FPRPLGITGL | SU27 | | 51 |
| FRLLDWQW | SU28 | | 52 |
| GFLG | SU29 | Lysosomal | 53 |
| | | Enzyme | |
| GGAANLVRGG | SU30 | MMP11 | 54 |
| GGGRR | SU31 | Urokinase | 55 |
| | | plasminogen | |
| | | activator (uPA) | |
| GGPRGLPG | SU32 | Cathepsin K | 56 |
| GGQPSGMWGW | SU33 | | 57 |
| GGSIDGR | SU34 | Factor Xa | 58 |
| GGWHTGRN | SU35 | | 59 |
| GIAGQ | SU36 | Collagenase | 60 |
| GKAFRR | SU37 | Kallikrein 2 | 61 |
| GPAGLYAQ | SU38 | | 62 |
| GPAGMKGL | SU39 | | 63 |
| GPEGLRVG | SU40 | Collagenase | 64 |
| GPLGIAGI | SU41 | Collagenase | 65 |
| GPLGVRG | SU42 | | 66 |
| GPQGIAGQ | SU43 | Collagenase | 67 |
| GPQGLLGA | SU44 | Collagenase | 68 |
| GPRSFG | SU45 | | 69 |
| GPRSFGL | SU46 | | 70 |
| GPSHLVLT | SU47 | | 71 |
| GVSQNYPIVG | SU48 | HIV Protease | 72 |
| GVVQASCRLA | SU49 | CMV Protease | 73 |
| GWEHDG | SU50 | Interleukin 1β | 74 |
| | | converting enzyme | |
| HSSKLQ | SU51 | Prostate Specific | 75 |
| | | Antigen | |
| HSSKLQEDA | SU52 | Prostate Specific | 76 |
| | | Antigen | |
| HSSKLQL | SU53 | Prostate Specific | 77 |
| | | Antigen | |
| HTGRSGAL | SU54 | | 78 |
| IDGR | SU55 | Factor Xa | 79 |
| IEGR | SU56 | Factor Xa | 80 |
| ILPRSPAF | SU57 | | 81 |
| IPVSLRSG | SU58 | MMP | 82 |
| ISSGL | SU59 | MMP | 83 |
| ISSGLL | SU60 | MMP | 84 |
| ISSGLLS | SU61 | MMP | 85 |
| ISSGLLSS | SU62 | MMP | 86 |
| ISSGLSS | SU63 | MMP | 87 |
| KGSGDVEG | SU64 | Caspase-3 | 88 |
| KQEQNPGST | SU65 | FAP | 89 |
| KRALGLPG | SU66 | MMP7 | 90 |
| LAAPLGLL | SU67 | | 91 |
| LAPLGLQRR | SU68 | | 92 |
| LAQKLKSS | SU69 | | 93 |
| LAQRLRSS | SU70 | | 94 |
| LEATA | SU71 | MMP9 | 95 |
| LKAAPRWA | SU72 | | 96 |
| LLAPSHRA | SU73 | | 97 |
| LPGGLSPW | SU74 | | 98 |
| LSGRSANI | SU75 | Serine protease | 99 |
| LSGRSANP | SU76 | Serine protease | 100 |
| LSGRSDDH | SU77 | Serine protease | 101 |
| LSGRSDIH | SU78 | Serine protease | 102 |
| LSGRSDNH | SU79 | Serine protease | 103 |
| LSGRSDNI | SU80 | Serine protease | 104 |
| LSGRSDNP | SU81 | Serine protease | 105 |
| LSGRSDQG | SU82 | Serine protease | 106 |
| LSGRSDQH | SU83 | Serine protease | 107 |
| LSGRSDTH | SU84 | Serine protease | 108 |
| LSGRSDYH | SU85 | Serine protease | 109 |
| LSGRSGNH | SU86 | Serine protease | 110 |
| LVLASSSFGY | SU87 | Herpes Simplex | 111 |
| | | Virus Protease | |
| MDAFLESS | SU88 | Collagenase | 112 |
| MGLFSEAG | SU89 | | 113 |
| MIAPVAYR | SU90 | | 114 |
| MVLGRSLL | SU91 | | 115 |
| NLL | SU92 | Cathepsin B | |
| NTLSGRSENHSG | SU93 | | 116 |
| NTLSGRSGNHGS | SU94 | | 117 |
| PAGLWLDP | SU95 | | 118 |
| PGGPAGIG | SU96 | | 119 |
| PIC(Et)FF | SU97 | Cathepsin D | 120 |
| PLGC(me)AG | SU98 | MMP | 121 |
| PLGL | SU99 | | 122 |
| PLGLAG | SU100 | MMP | 123 |
| PLGLAX | SU101 | MMP | 124 |
| PLGLWA | SU102 | MMP | 125 |
| PLGLWSQ | SU103 | MMP | 126 |
| PLTGRSGG | SU104 | | 127 |
| PMAKK | SU105 | | 128 |
| PPRSFL | SU106 | Thrombin | 129 |
| PR(S/T)(L/I)(S/T) | SU107 | MMP9 | |
| PRFRIIGG | SU108 | Plasmin | 130 |
| PVGYTSSL | SU109 | | 131 |
| PVQPIGPQ | SU110 | Collagenase | 132 |
| QALAMSAI | SU111 | Collagenase | 133 |
| QGRAITFI | SU112 | | 134 |
| QNQALRMA | SU113 | | 135 |
| RGPA | SU114 | | 136 |
| RGPAFNPM | SU115 | | 137 |
| RGPATPIM | SU116 | | 138 |
| RKSSIIIRMRDVVL | SU117 | Plasmin | 139 |
| RLQLKAC | SU118 | MMP | 140 |
| RLQLKL | SU119 | MMP | 141 |
| RMHLRSLG | SU120 | | 142 |
| RPSPMWAY | SU121 | | 143 |
| RQARVVNG | SU122 | Matriptase | 144 |
| SAGFSLPA | SU123 | | 145 |
| SAPAVESE | SU124 | Collagenase | 146 |
| SARGPSRW | SU125 | | 147 |
| SGEPAYYTA | SU126 | | 148 |
| SGGPLGVR | SU127 | | 149 |
| SGRIGFLRTA | SU128 | MMP14 | 150 |
| SGRSA | SU129 | Urokinase | 151 |
| | | plasminogen | |
| | | activator (uPA) | |
| SGRSANPRG | SU130 | | 152 |
| SMLRSMPL | SU131 | | 153 |
| SPLPLRVP | SU132 | | 154 |
| SPLTGRSG | SU133 | | 155 |
| SPRSIMLA | SU134 | | 156 |
| SSRGPAYL | SU135 | | 157 |
| SSRHRRALD | SU136 | Plasmin | 158 |
| SSSFDKGKYKKGDDA | SU137 | Plasmin | 159 |
| SSSFDKGKYKRGDDA | SU138 | Plasmin | 160 |
| STFPFGMF | SU139 | | 161 |
| TARGPSFK | SU140 | | 162 |
| TGRGPSWV | SU141 | | 163 |
| TSGRSANP | SU142 | | 164 |
| TSTSGRSANPRG | SU143 | | 165 |
| VAGRSMRP | SU144 | | 166 |
| VAQFVLTE | SU145 | Collagenase | 167 |
| VHMPLGFLGP | SU146 | | 168 |
| VPLSLYSG | SU147 | MMP9 | 169 |
| VVPEGRRS | SU148 | | 170 |
| WATPRPMR | SU149 | | 171 |
| YGAGLGVV | SU150 | Collagenase | 172 |
| HPVGLLAR | SU151 | | 173 |

### 6.4.3. Spacers

Exemplary spacer sequences that can be incorporated into the protease-cleavable linkers are set forth in Table C below. In addition to the spacer sequences set forth in Table C, any of the non-cleavable linker sequences described in Section 6.5, e.g., the non-cleavable linker sequences set forth in Table E, or portions thereof can be used as spacer sequences. In some embodiments, spacer sequences are absent entirely from the protease-cleavable linkers.

TABLE C
Spacer Sequences for Protease-Cleavable Linkers

| Spacer Sequence | Designation | SEQ ID NO: |
|---|---|---|
| (GGGGS)ₙ | SP1 | 174 |
| (GGGS)ₙ | SP2 | 175 |
| (GGS)ₙ | SP3 | 176 |
| (GS)ₙ | SP4 | 177 |
| (GSGGS)ₙ | SP5 | 178 |
| GGGGSGGGGS | SP6 | 179 |
| GGGGSGGGGSGGGGS | SP7 | 180 |
| GGGGSGGGGSGGGGSGGGGS | SP8 | 181 |
| GGGKSGGGKSGGGKS | SP9 | 182 |
| GGGKSGGKGSGKGGS | SP10 | 183 |
| GGGS | SP11 | 184 |
| GGGSG | SP12 | 185 |
| GGKGSGGKGSGGKGS | SP13 | 186 |
| GGSGGGGSGGGGS | SP14 | 187 |
| GGSGGS | SP15 | 188 |
| GGSGGSGGSGS | SP16 | 189 |
| GSGGG | SP17 | 190 |
| GSGSG | SP18 | 191 |
| GSS | SP19 | |
| GSSG | SP20 | 192 |
| GSSGGSGGSG | SP21 | 193 |
| GSSGGSGGSGG | SP22 | 194 |
| GSSGGSGGSGGS | SP23 | 195 |
| GSSGGSGGSGGSG | SP24 | 196 |
| GSSGGSGGSGGSGGGS | SP25 | 197 |
| GSSGGSGGSGS | SP26 | 198 |
| GSSGT | SP27 | 199 |
| GSSSG | SP28 | 200 |
| QGQSGQ | SP29 | 201 |
| QGQSGQG | SP30 | 202 |
| QGQSGS | SP31 | 203 |
| QSGQ | SP32 | 204 |
| QSGQG | SP33 | 205 |
| QSGS | SP34 | 206 |
| SGQ | SP35 | |
| SGQG | SP36 | 207 |
| SGS | SP37 | |
| (G)ₙ | SP38 | 208 |

In some embodiments, as used in Table C above, n is an integer from 1 to 10, e.g., 1, 2, 3, 4, 5, 6, 7, 8, 9 or 10.

### 6.4.4. Exemplary Protease-Cleavable Linkers

Exemplary protease-cleavable linkers comprising one or more substrate sequences as well as spacer sequences are set forth in Table D below.

TABLE D
Protease-Cleavable Linker Sequences

| Linker Sequence | Designation | Cleaving Protease(s) | SEQ ID NO: |
|---|---|---|---|
| GGGISSGLLSGRSDNHGGGISSG | PCL1 | | 209 |
| LLSGRSDNHGGS | | | |
| GGGISSGLLSGRSDNHGGGISSG | PCL2 | | 209 |
| LLSGRSDNHGGS | | | |
| GGSGGSIPVSLRSGGGISSGLLS | PCL3 | | 210 |
| GRSDNHGGSGGS | | | |
| GGSGGSVPLSLYSGGGISSGLLS | PCL4 | | 211 |
| GRSDNHGGSGGS | | | |
| GGSHPVGLLARGGGHPVGLLAR | PCL5 | | 212 |
| GGGHPVGLLARGS | | | |
| GGSHPVGLLARGGGHPVGLLAR | PCL6 | | 213 |
| GGSGRSAGGSGRSA | | | |
| AVGLLAPPGGLSGRSANI | PCL7 | ADAM17_2, FAPa_1, | 214 |
| | | CTSL1_1 | |
| AVGLLAPPGGLSGRSANP | PCL8 | FAPa_1, ADAM17_2, | 215 |
| | | CTSL1_1 | |
| AVGLLAPPGGLSGRSDDH | PCL9 | MMP14_1, MMP14_1, | 216 |
| | | MMP14_1 | |
| AVGLLAPPGGLSGRSDIH | PCL10 | MMP14_1, MMP14_1, | 217 |
| | | MMP14_1 | |
| AVGLLAPPGGLSGRSDNH | PCL11 | MMP14_1, MMP14_1 | 218 |
| AVGLLAPPGGLSGRSDNI | PCL12 | MMP14_1, CTSL1_1, | 219 |
| | | ADAM17_2 | |
| AVGLLAPPGGLSGRSDNP | PCL13 | CTSL1_1, ADAM17_2, | 220 |
| | | FAPa_1 | |
| AVGLLAPPGGLSGRSDQH | PCL14 | | 221 |
| AVGLLAPPGGLSGRSDTH | PCL15 | FAPa_1, CTSL1_1, | 222 |
| | | ADAM17_2 | |
| AVGLLAPPGGLSGRSDYH | PCL16 | | 223 |
| AVGLLAPPGGTSTSGRSANPRG | PCL17 | | 224 |
| AVGLLAPPSGRSANPRG | PCL18 | | 225 |
| AVGLLAPPTSGRSANPRG | PCL19 | | 226 |
| GGALFKSSFPGPAGLYAQPLAQK | PCL20 | CTSL1_1, MMP14_1, | 227 |
| LKSSGGK | | ADAM17_2 | |
| GGGGSGGGGSGGGGSFVGGTG | PCL21 | | 228 |
| GGGSGGGGSGGS | | | |
| GGGGSGGGGSGGGGSISSGLLS | PCL22 | | 229 |
| GRSDNHGGSGGS | | | |
| GGGGSGGGGSGGGGSVPLSLYS | PCL23 | | 230 |
| GGGSGGSGGSGS | | | |
| GGGGSGGGGSGPLGLWSQGGG | PCL24 | | 231 |
| GSGGGGSGGGGSGG | | | |
| GGGGSGGGGSKKAAPGGGGSG | PCL25 | | 232 |
| GGGSGGGGSGGS | | | |
| GGGGSGGGGSKKAAPVNGGGG | PCL26 | | 233 |
| GSGGGGSGGGGS | | | |
| GGGGSGGGGSPMAKKGGGGSG | PCL27 | | 234 |
| GGGSGGGGSGGS | | | |
| GGGGSGGGGSPMAKKVNGGGG | PCL28 | | 235 |
| GSGGGGSGGGGS | | | |
| GGGGSGGGGSQARAKGGGGSG | PCL29 | | 236 |
| GGGSGGGGSGGS | | | |
| GGGGSGGGGSQARAKVNGGGG | PCL30 | | 237 |
| GSGGGGSGGGGS | | | |
| GGGGSGGGGSRQARVVNGGGG | PCL31 | | 238 |
| GSGGGGSGGGGS | | | |
| GGGGSGGGGSRQARVVNGGGG | PCL32 | | 239 |
| GSVPLSLYSGGGGGSGGGGS | | | |
| GGGGSGGGGSRQARVVNSVPLS | PCL33 | | 240 |
| LYSGGGGGSGGGGS | | | |
| GGGGSGGGGSVHMPLGFLGPG | PCL34 | | 241 |
| GGGSGGGGSGGS | | | |
| GGGGSVHMPLGFLGPGRSRGSF | PCL35 | | 242 |
| PGGGGS | | | |
| GGGGSVHMPLGFLGPPMAKKGG | PCL36 | | 243 |
| GGSGGGGSGGS | | | |
| GGGGSVHMPLGFLGPRQARVVN | PCL37 | | 244 |
| GGGGSGGGGS | | | |
| GGGGSVHMPLGFLGPRQARVVN | PCL38 | | 245 |
| GGGGGGGGSGG | | | |
| GGPLAQKLKSSALFKSSFPGPAG | PCL39 | ADAM17_2, CTSL1_1, | 246 |
| LYAQGGR | | MMP14_1 | |
| GLSGRSDNHGGAVGLLAPP | PCL40 | | 247 |
| GLSGRSDNHGGVHMPLGFLGP | PCL41 | | 248 |
| ISSGLLSGRSANI | PCL42 | MMP, Serine protease | 249 |
| ISSGLLSGRSANP | PCL43 | MMP, Serine protease | 250 |
| ISSGLLSGRSANPRG | PCL44 | MMP, Serine protease | 251 |
| ISSGLLSGRSDDH | PCL45 | MMP, Serine protease | 252 |
| ISSGLLSGRSDIH | PCL46 | MMP, Serine protease | 253 |
| ISSGLLSGRSDNH | PCL47 | MMP, Serine protease | 254 |
| ISSGLLSGRSDNI | PCL48 | CTSL1_1, MMP14_1 | 255 |
| ISSGLLSGRSDNP | PCL49 | MMP, Serine protease | 256 |
| ISSGLLSGRSDQH | PCL50 | MMP, Serine protease | 257 |
| ISSGLLSGRSDTH | PCL51 | MMP, Serine protease | 258 |
| ISSGLLSGRSDYH | PCL52 | MMP, Serine protease | 259 |
| ISSGLLSGRSGNH | PCL53 | MMP, Serine protease | 260 |
| ISSGLLSSGGSGGSLSGRSDNH | PCL54 | | 261 |
| ISSGLLSSGGSGGSLSGRSGNH | PCL55 | | 262 |
| KGGPGGPAGIGPLAQRLRSSALF | PCL56 | FAPa_1, ADAM17_1, | 263 |
| KSSFPGR | | CTSL1_1 | |
| KSGPGGPAGIGALFFSSPPLAQKL | PCL57 | FAPa_1, CTSL1_2, | 264 |
| KSSGGR | | ADAM17_2 | |
| LSGRSDNHGGAVGLLAPP | PCL58 | | 265 |
| LSGRSDNHGGSGGSISSGLLSS | PCL59 | | 266 |
| LSGRSDNHGGSGGSQNQALRMA | PCL60 | | 267 |
| LSGRSDNHGGVHMPLGFLGP | PCL61 | | 268 |
| LSGRSGNHGGSGGSISSGLLSS | PCL62 | | 269 |
| LSGRSGNHGGSGGSQNQALRMA | PCL63 | | 270 |
| QNQALRMAGGSGGSLSGRSDNH | PCL64 | | 271 |
| QNQALRMAGGSGGSLSGRSGNH | PCL65 | | 272 |
| RGGALFKSSFPLAQKLKSSGPAG | PCL66 | CTSL1_1, ADAM17_2, | 273 |
| LYAQGGK | | MMP14_1 | |
| RGGGPAGLYAQPLAQKLKSSALF | PCL67 | MMP14_1, ADAM17_2, | 274 |
| KSSFPGG | | CTSL1_1 | |
| SGGFPRSGGSFNPRTFGSKRKR | PCL68 | thrombin, factor Xa, | 275 |
| RGSRGGGG | | hepsin | |
| SGPLAQKLKSSGPAGLYAQALFK | PCL69 | ADAM17_2, MMP14_1, | 276 |
| SSFPGSK | | CTSL1_1 | |
| TSTSGRSANPRGGGAVGLLAPP | PCL70 | | 277 |
| TSTSGRSANPRGGGVHMPLGFL | PCL71 | | 278 |
| GP | | | |
| VHMPLGFLGPGGLSGRSDNH | PCL72 | | 279 |
| VHMPLGFLGPGGTSTSGRSANP | PCL73 | | 280 |
| RG | | | |
| SGRSAGGGSGRSAGGGSGRSA | PCL74 | uPA | 281 |
| HPVGLLARGGGHPVGLLARGGG | PCL75 | MPA (MMP-2 and uPA) | 282 |
| SGRSAGGGSGRSA | | | |
| GPLGVRGK | PCL76 | MMP-2 | 283 |
| HPVGLLAR | PCL77 | MMP-2 | 173 |
| GPQGIAGQ | PCL78 | MMP-2, MMP-9, and to | 67 |
| | | some degree MT1-MMP | |
| VPMSMRGG | PCL79 | MMP-9 and MMP-2 | 284 |
| IPVSLRSG | PCL80 | MMP-2, and to some | 82 |
| | | degree MMP-9 or MMP- | |
| | | 7 | |
| RPFSMIMG | PCL81 | MMP-9 and MMP-7, to | 285 |
| | | some degree MMP-3 | |
| VPLSLTMG | PCL82 | MMP-7, to some degree | 286 |
| | | MMP-9, MMP-2, MPT-1- | |
| | | MMP | |
| VPLSLYSG | PCL83 | MMP-2, MMP-9, MMP-7 | 169 |
| IPESLRAG | PCL84 | MMP-2, MMP-7, MMP-9, | 287 |
| | | to some degree MPT-1- | |
| | | MMP | |
| VPLSLYSGGGISSGLLSGRSDNH | PCL85 | | 288 |
| GGGISSGLLSGRSDNHGGGS | PCL86 | | 289 |
| GGGHPVGLLARGGGS | PCL87 | | 290 |
| GGGSGGGSGGGGISSGLLSGRS | PCL88 | | 291 |
| DNHGGGSGGGSGGS | | | |
| GGGGISSGLLSGRSDNHGGGISS | PCL89 | | 292 |
| GLLSGRSDNHGGS | | | |
| GGGSGGSIPVSLRSGGGISSGLL | PCL90 | | 293 |
| SGRSDNHGGSGGS | | | |
| GGGSGGSVPLSLYSGGGISSGLL | PCL91 | | 294 |
| SGRSDNHGGSGGS | | | |
| GGGSHPVGLLARGGGHPVGLLA | PCL92 | | 295 |
| RGGGHPVGLLARGS | | | |
| GGGSHPVGLLARGGGHPVGLLA | PCL93 | | 296 |
| RGGSGRSAGGSGRS | | | |
| GISSGLLSGRSDNHG | PCL94 | | 297 |
| GGGSISSGLLSGRSDNHGGGS | PCL95 | | 298 |

In certain aspects, the protease-cleavable linker comprises an amino acid sequence having up to 5, up to 4, up to 3, up to 2 or up to 1 amino acid substitution(s) as compared to the sequence set forth in Table D. Thus, in some embodiments, the protease-cleavable linker comprises or consists of any amino acid sequence in Table D with 1-5 amino acid substitutions as compared to the sequence set forth in Table D.

### 6.5. Non-Cleavable Linkers

In certain aspects, the present disclosure provides IFN proproteins in which two or more components of an IFN proprotein are connected to one another by a peptide linker. By way of example and not limitation, linkers can be used to connect an Fc domain and a targeting moiety or different domains within a targeting moiety (e.g., VH and VL domains in an scFv).

Preferably, all linkers in the IFN proprotein other than the protease-cleavable linkers whose cleavage results in activation of IFN are non-cleavable linkers (NCLs).

A non-cleavable linker can range from 2 amino acids to 60 or more amino acids, and in certain aspects a non-cleavable peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a non-cleavable linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the non-cleavable linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the non-cleavable linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the non-cleavable linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible non-cleavable linkers are particularly preferred.

Examples of flexible non-cleavable linkers that can be used in the IFN proproteins of the disclosure include those disclosed by Chen et al., 2013, Adv Drug Deliv Rev. 65 (10): 1357-1369 and Klein et al., 2014, Protein Engineering, Design & Selection 27 (10): 325-330. Particularly useful flexible non-cleavable linkers are or comprise repeats of glycines and serines, e.g., a monomer or multimer of GₙS (SEQ ID NO: 299) or SGₙ (SEQ ID NO: 300), where n is an integer from 1 to 10, e.g., 12, 3, 4, 5, 6, 7, 8, 9 or 10. In one embodiment, the non-cleavable linker is or comprises a monomer or multimer of repeat of G₄S (SEQ ID NO: 301) e.g., (GGGGS)ₙ (SEQ ID NO: 301).

Polyglycine non-cleavable linkers can suitably be used in the IFN proproteins of the disclosure. In some embodiments, a peptide non-cleavable linker comprises two consecutive glycines (2Gly), three consecutive glycines (3Gly), four consecutive glycines (4Gly (SEQ ID NO: 302)), five consecutive glycines (5Gly (SEQ ID NO: 303)), six consecutive glycines (6Gly (SEQ ID NO: 304)), seven consecutive glycines (7Gly (SEQ ID NO: 305)), eight consecutive glycines (8Gly (SEQ ID NO: 306)) or nine consecutive glycines (9Gly (SEQ ID NO: 307)).

Exemplary non-cleavable linker sequences are set forth in Table E below.

TABLE E
Non-Cleavable Linker Sequences

| | | SEQ ID |
|---|---|---|
| Linker Sequence | Designation | NO: |
| GGGGSLALGPGGGGGSLALGPGGGGGSLALGPGGS | NCL1 | 308 |
| GGGGSGGGGSGGGGSGGGGSGGGGS | NCL2 | 309 |
| (GGGGS)ₙ, optionally where n = 1-10, | NCL3 | 174 |
| e.g., 1, 2, 3, 4, 5, 6, 7, 8, 9, or 10 | | |
| (GGGS)ₙ, optionally where n = 1-10, | NCL4 | 175 |
| e.g., 1, 2, 3, 4, 5, 6, 7, 8, 9, or 10 | | |
| (GGS)ₙ, optionally where n = 1-10, | NCL5 | 176 |
| e.g., 1, 2, 3, 4, 5, 6, 7, 8, 9, or 10 | | |
| (GS)ₙ, optionally where n = 1-10, | NCL6 | 177 |
| e.g., 1, 2, 3, 4, 5, 6, 7, 8, 9, or 10 | | |
| (GSGGS)ₙ, optionally where n = 1-10, | NCL7 | 178 |
| e.g., 1, 2, 3, 4, 5, 6, 7, 8, 9, or 10 | | |
| ADAAP | NCL8 | 310 |
| ADAAPTVSIFP | NCL9 | 311 |
| ADAAPTVSIFPP | NCL10 | 312 |
| AKTTAP | NCL11 | 313 |
| AKTTAPSVYPLAP | NCL12 | 314 |
| AKTTPKLEEGEFSEARV | NCL13 | 315 |
| AKTTPKLGG | NCL14 | 316 |
| AKTTPP | NCL15 | 317 |
| AKTTPPSVTPLAP | NCL16 | 318 |
| ASTKGP | NCL17 | 319 |
| ASTKGPSVFPLAPASTKGPSVFPLAP | NCL18 | 320 |
| EGKSSGSGSESKST | NCL19 | 321 |
| GEGESGEGESGEGES | NCL20 | 322 |
| GEGESGEGESGEGESGEGES | NCL21 | 323 |
| GEGGSGEGGSGEGGS | NCL22 | 324 |
| GENKVEYAPALMALS | NCL23 | 325 |
| GGEGSGGEGSGGEGS | NCL24 | 326 |
| GGGESGGEGSGEGGS | NCL25 | 327 |
| GGGESGGGESGGGES | NCL26 | 328 |
| GGGGSGGGGS | NCL27 | 179 |
| GGGGSGGGGSGGGGS | NCL28 | 180 |
| GGGGSGGGGSGGGGSGGGGS | NCL29 | 181 |
| GGGKSGGGKSGGGKS | NCL30 | 182 |
| GGGKSGGKGSGKGGS | NCL31 | 183 |
| GGGS | NCL32 | 184 |
| GGGSG | NCL33 | 185 |
| GGKGSGGKGSGGKGS | NCL34 | 186 |
| GGSG | NCL35 | 329 |
| GGSGG | NCL36 | 330 |
| GGSGGGGSG | NCL37 | 331 |
| GGSGGGGSGGGGS | NCL38 | 187 |
| GHEAAAVMQVQYPAS | NCL39 | 332 |
| GKGGSGKGGSGKGGS | NCL40 | 333 |
| GKGKSGKGKSGKGKS | NCL41 | 334 |
| GKGKSGKGKSGKGKSGKGKS | NCL42 | 335 |
| GKPGSGKPGSGKPGS | NCL43 | 336 |
| GKPGSGKPGSGKPGSGKPSGS | NCL44 | 337 |
| GPAKELTPLKEAKVS | NCL45 | 338 |
| GSAGSAAGSGEF | NCL46 | 339 |
| GSGGG | NCL47 | 190 |
| GSGSG | NCL48 | 191 |
| GSS | NCL49 | |
| GSSG | NCL50 | 192 |
| GSSGGSGGSG | NCL51 | 193 |
| GSSGGSGGSGG | NCL52 | 194 |
| GSSGGSGGSGGS | NCL53 | 195 |
| GSSGGSGGSGGSG | NCL54 | 196 |
| GSSGGSGGSGGSGGGS | NCL55 | 197 |
| GSSGGSGGSGS | NCL56 | 198 |
| GSSGT | NCL57 | 199 |
| GSSSG | NCL58 | 200 |
| GSTSGSGKPGSGEGSTKG | NCL59 | 340 |
| GTAAAGAGAAGGAAAGAAG | NCL60 | 341 |
| GTSGSSGSGSGGSGSGGGG | NCL61 | 342 |
| IRPRAIGGSKPRVA | NCL62 | 343 |
| KESGSVSSEQLAQFRSLD | NCL63 | 344 |
| KTTPKLEEGEFSEAR | NCL64 | 345 |
| PRGASKSGSASQTGSAPGS | NCL65 | 346 |
| QPKAAP | NCL66 | 347 |
| QPKAAPSVTLFPP | NCL67 | 348 |
| RADAAAA(G4S)₄ | NCL68 | 349 |
| RADAAAAGGPGS | NCL69 | 350 |
| RADAAP | NCL70 | 351 |
| RADAAPTVS | NCL71 | 352 |
| SAKTTP | NCL72 | 353 |
| SAKTTPKLEEGEFSEARV | NCL73 | 354 |
| SAKTTPKLGG | NCL74 | 355 |
| STAGDTHLGGEDFD | NCL75 | 356 |
| TVAAP | NCL76 | 357 |
| TVAAPSVFIFPP | NCL77 | 358 |
| TVAAPSVFIFPPTVAAPSVFIFPP | NCL78 | 359 |
| AGSGNSSGSGGSGGSGNSSGSGGSPVPSTPPTPSPSTPPTPSPSAS | NCL79 | 360 |
| GGGGSAS | NCL80 | 361 |
| GGGGSGGGGSAS | NCL81 | 362 |
| GGGGSGGGGSGGGGSAS | NCL82 | 363 |
| GGGGGGGGSGGGGSGGGGSGGGGSAS | NCL83 | 364 |
| GGGGSGGGGSGGGGGGGGSGGGGSGGGGSAS | NCL84 | 365 |
| AGGGSGGGGSGGGGSGGGGSGGGGSGGGGSGGGGSAS | NCL85 | 366 |
| AGGGSGGGGSGGGGSGGGGSGGGGSGGGGSGGGGSGGGGSAS | NCL86 | 367 |
| AGGGSGGGGSGGGGSGGGGSGGGGSGGGGSGGGGSGGGGSGGG | NCL87 | 368 |
| GSAS | | |

In certain aspects, the IFN proprotein of the disclosure may comprise a polypeptide chain comprising, in an N- to C-terminal orientation, a targeting moiety (or targeting moiety chain), a hinge domain, a CH2 domain, and a CH3 domain (e.g., as depicted in FIG. 1A) or a polypeptide chain comprising a targeting moiety (or targeting moiety chain), a hinge domain, followed by a protease cleavable linker (e.g., as depicted in FIG. 1C). Thus, the hinge domain can be said to constitute a type of linker. Exemplary hinge domains are set forth in Section 6.8.3.

### 6.6. Targeting Moiety

The incorporation of targeting moieties in the IFN proproteins of the disclosure permits the delivery of high concentrations of IFN into the tumor microenvironment with a concomitant reduction of systemic exposure, resulting in fewer side effects than obtained with untargeted IFN molecules.

It is anticipated that any type of target molecule present or capable of driving the IFN proprotein at a particular locale or tissue may be targeted by the IFN proproteins of the disclosure. In some embodiments, the IFN proproteins are intended to treat cancer, e.g., by inducing a local immune response against tumor tissue. Accordingly, the targeting molecule can be any local tumor and associated target molecule. The target molecules recognized by the targeting moieties of the IFN proproteins of the disclosure are generally found, for example, on the surfaces of activated T cells, on the surfaces of tumor cells, on the surfaces of dendritic or other antigen-presenting cells, on the surfaces of natural killer (NK) cells, on the surfaces of virus-infected cells, on the surfaces of other diseased cells, free in blood serum, in the extracellular matrix (ECM), or immune cells present in the target site, e.g., tumor reactive lymphocytes.

In various embodiments, the target molecule is an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA), a dendritic cell (DC) or other antigen-presenting cell (APC) antigen, or a natural killer (NK) cell antigen. The skilled artisan would recognize that the foregoing categories of target molecules are not mutually exclusive and thus a given target molecule may fall into more than one of the foregoing categories of target molecules. For example, some molecules may be considered both TAAs and ECM proteins, and other molecules may be considered both TCAs and checkpoint inhibitors.

Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Non-limiting examples of ECM antigens include syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

Other target molecules are cell surface molecules of tumor or viral lymphocytes, for example T-cell co-stimulatory proteins such as CD27, CD28, 4-1BB (CD137), OX40, CD30, CD40, ICOS, lymphocyte function-associated antigen-1 (LFA-1), CD2, CD7, LIGHT, NKG2C, and B7-H3.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In other embodiments, the target molecule is LAG3. In yet other embodiments the target molecule is PDL1.

In certain embodiments, the target molecules are on the surfaces of dendritic cells or other antigen-presenting cells, such as XCR1, Clec9a, CD1c, CD11c, CD14, PDL1, macrophage mannose receptor (CD206), and DEC-205.

In further embodiments, the target molecules are on the surfaces of natural killer (NK) cells such as CD335, CD38, CD2, NKG2D, NKp44, NKp30, CD16, LFA-1, CD27, KIR, NKH1A, and NKp46.

The antibodies and antigen-binding portions generally bind to specific antigenic determinants and are able to direct the IFN proprotein to a target site, for example to a specific type of tumor cell or tumor stroma that bears the antigenic determinant. In particular embodiments, the targeting moiety recognizes a tumor-associated antigen (TAA). Preferably, the TAA is a human TAA. The antigen may or may not be present on normal cells. In certain embodiments, the TAA is preferentially expressed or upregulated on tumor cells as compared to normal cells. In other embodiments, the TAA is a lineage marker. Exemplary TAAs include Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R-(IL6 receptor), CD20, MCSP, PDGFBR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostase specific antigen (PSA), PAP, LAGA-1a, p53, prostein, PSMA, surviving and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, the extra domain A (EDA) and extra domain B (EDB) of fibronectin and the A1 domain of tenascin-C (TnC A1).

Suitable targeting moiety formats are described in Section 6.7. The targeting moiety is preferably an antigen binding moiety, for example an antibody or an antigen-binding portion of an antibody, e.g., an scFv, as described in Section 6.7.2 or a Fab, as described in Section 6.7.1.

In some embodiments, the targeting moieties target the exemplary target molecules set forth in Table F below, together with references to exemplary antibodies or antibody sequences upon which the targeting moiety can be based.

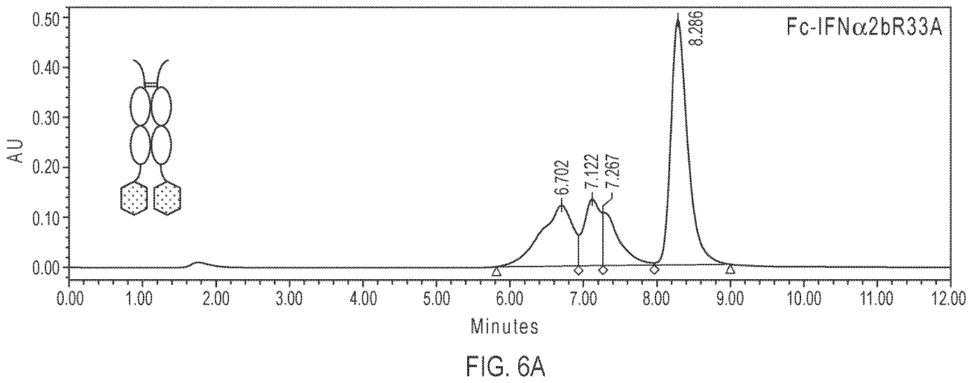

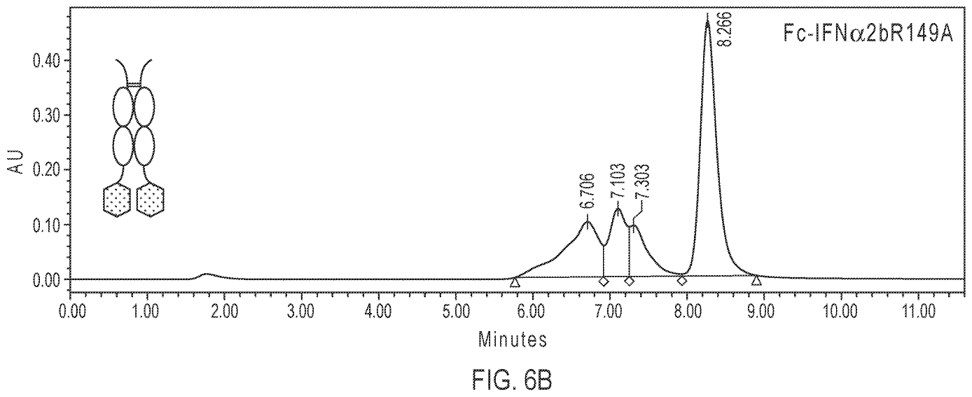

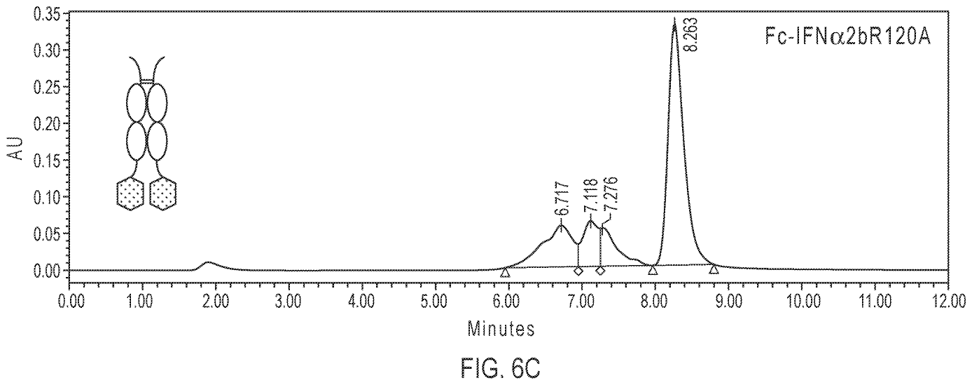

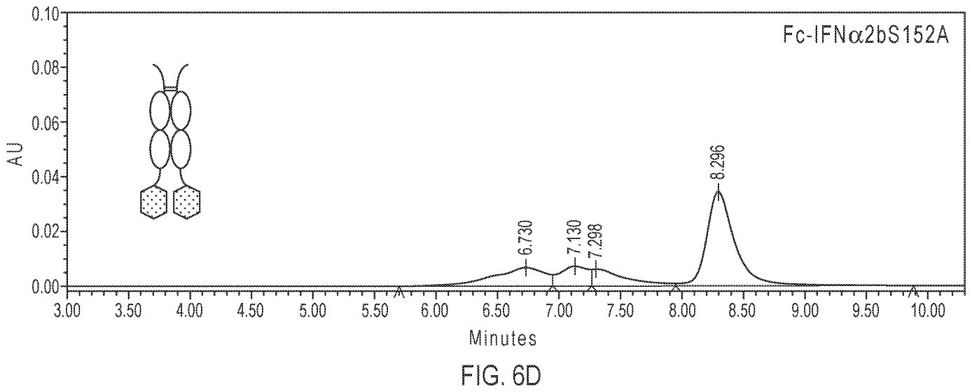

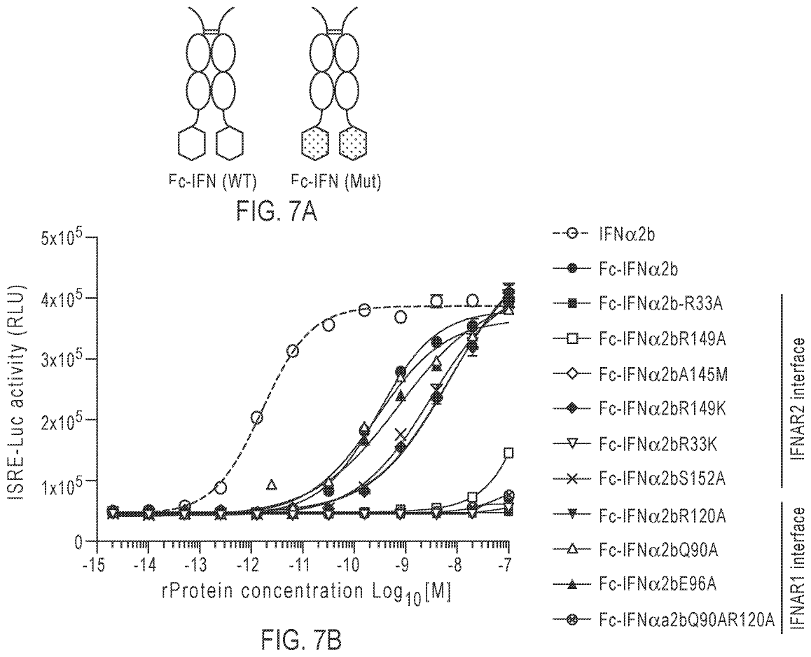

TABLE F
Exemplary Target Molecules

| Target | Antibody Name and/or Binding Sequences |
|---|---|
| 1-92-LFA-3 | Amevive ™ (alefacept) |
| 5T4 | GEN1044 |
| Activin Receptor Type II | Bimagrumab |
| | VH: SEQ ID NOs: 107, 109 of U.S. Pat. No. 8,388,968 B2 |
| | VL: SEQ ID NOs: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID |
| | NO: 21, 26 or 31 of US 2021/0171641 A1. |
| | VL: the VL sequence of the light chain of SEQ ID NO: 20, |
| | 22 or 30 of US 2021/0171641 A1. |
| B7-H3 (CD276) | VH: the VH sequence of the heavy chain of SEQ ID |
| | NO: 21, 29 or 37 of US 2019/0002563 A1. |
| | VL: the VL sequence of the light chain of SEQ ID NO: 17, |
| | 25 or 33 of US 2019/0002563 A1. |
| B7-H3 (CD276) | VH: the VH sequence of the heavy chain of SEQ ID |
| | NO: 146, 147 or 148 of U.S. Pat. No. 10,640,563. |
| | VL: the VL sequence of the light chain of SEQ ID NO: 143, |
| | 144 or 145 of U.S. Pat. No. 10,640,563. |
| BAFF/B Lymphocyte | Benlysta ™ (velimumab) |
| Stimulator | |
| BAFF/B Lymphocyte | VH: amino acids 1-123 of SEQ ID NO: 327 of |
| Stimulator | U.S. Pat. No. 7,138,501 |
| | VL: amino acids 139-249 of SEQ ID NO: 327 of |
| | U.S. Pat. No. 7,138,501. |
| BAFF/B Lymphocyte | VH: amino acids 1-126 of SEQ ID NO: 1321 of |
| Stimulator | U.S. Pat. No. 7,605,236; |
| | VL: amino acids 143-251 of SEQ ID NO: 1049 of |
| | U.S. Pat. No. 7,605,236. |
| BAFF/B Lymphocyte | Belimumab |
| Stimulator | |
| BCMA | VH: the VH sequence of the heavy chain of SEQ ID NO. |
| | 126 of US 2021/0206865 A1 |
| | VL: the VL sequence of the light chain of SEQ ID NO. 129 |
| | or SEQ ID NO. 132 of US 2021/0206865 A1 |
| CA125 | Igobumab |
| CA125 | OvaRex ™ (oregobumab) |
| Cadherin | The antibodies described in US Pub. No. US |
| | 2006/0039915. |
| N-cadherin | An antibody that binds to the amino acid sequence of |
| | SEQ ID NO: 10, 17 or 18 of US Pub. No. US |
| | 2010/0278821. |
| CD11a | Raptiva ™ (efalizumab) |
| | Sequence in Werther et al., 1996, The Journal of |
| | Immunology 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID |
| | NO: 124 of US Patent Pub. US 2017/0002060 A1 |
| | VL: the VL sequence of the light chain of SEQ ID NO: 125 |
| | of US Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD38 | IB4, HB7 CS/2, clone 90 and NIM-R5 as disclosed in PCT |
| | Pub. WO2015/009726A2 and references cited therein. |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US 2017/0002060 |
| | A1 |
| | VL: SEQ ID NO: 2 of US Patent Pub. US 2017/0002060 |
| | A1 |
| CD70 | Blenrep ™ (borsetuzumab mafodotin) |
| CD123 | Flotetuzumab |
| CD206 | Anti-CD206 antibodies having a VH a of SEQ ID No. 2 |
| | and a VL of SEQ ID NO: 4 of WO2003/040169A2 |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID |
| | Nps: 4-6 of U.S. Pat. No. 8,771,690 B2 |
| | CDR-L1, CDR-L2 and CDR-L3 sequences of SEQ ID |
| | Nps: 1-3 of U.S. Pat. No. 8,771,690 B2 |
| CEA | CEA binding portion of RO6958688/RG7802 from clinical |
| | trial NCT02324257 |
| CEA | Cibisatamab |
| CEA | CEA binding portion of MEDI-565/MT110/AMG211 from |
| | clinical trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO |
| | 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Clec9a | Anti-Clec9a antibodies having VH and VL amino acids of |
| | SEQ ID Nos. 43 and 48 of PCT Pub. No. |
| | WO2009/026660A1 |
| Clec9a | Anti-Clec9a antibodies having a VH a of SEQ ID NO. 38 |
| | and a VL of SEQ ID NO: 37 or SEQ ID NO: 43 of PCT Pub. |
| | No. WO 2022/073062A1 |
| | Anti-Clec9a antibodies having a VH a of SEQID NO. 8 |
| | and a VL of SEQ ID NO. 7 of PCT Pub. No. |
| | WO2022/073062A1 |
| Collagen alpha-4 chain | TRC093 (MT293) |
| Collagen | The collagen binding antibody fragment described in |
| | Liang et al., 2016, Sci. Rep. 5, 18205; doi: |
| | 10.1038/srep18205 (2016). |
| Collagen type I | Cetuximab (Erbitux) |
| Collagen type X | The amino acid sequences of SEQ ID NO: 1 or 2 of PCT |
| | Pub No. WO 2019/020797. |
| Collagen type X | The amino acid sequences of SEQ ID NO: 1 of PCT Pub |
| | No. WO 2014/180992. |
| Collagen type X | Antibody X34 as described in I. Girkontaite et al., |
| | “Immunolocalization of type X collagen in normal fetal and |
| | adult osteoarthritic cartilage with monoclonal |
| | antibodies,” Matrix Biol 15, 231-238 (1996). |
| Collagen type X | Antibodies X53 or 1H8 or ARC0659 or JF0961 collagen X |
| | polyclonal antibody sold under catalog number PA5- |
| | 115039 or PA5-116871 or PA5-97603 or PA5-49198 from |
| | ThermoFisher Scientific. |
| Collagen type X | Antibody sold under catalog number RDI-COLL10abr from |
| | RDI. |
| Complement C5 | Soliris ™ (eculizumab) |
| | VH: amino acids 1-122 of SEQ ID NO: 10 of U.S. Pat. |
| | No. 6,355,245 |
| | VL: amino acids 3-110 of SEQ ID NO: 9 of U.S. Pat. |
| | No. 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DEC-205 | Anti-DEC-205 antibodies having a VH/VL pair of SEQ ID |
| | NOS. 4/10, 16/22, 28/34, 40/46, 52/58, and 76/82 of PCT |
| | Pub. WO2009/061996A2 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | Mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of US Patent Pub. No. US |
| | 2020/0317806 A1 |
| | HL: SEQ ID NO: 3 of US Patent Pub. No. US |
| | 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™(oportuzumab monatox) |
| EpCAM | M701 |
| F protein of RSV | Synagic ™ (palivizumab) |
| GD2 | 3F8 |
| Glycoprotein receptor IIb/IIIa | ReoPro ™ (abiciximab) |
| gpA33 | MGD007 |
| GPC3 | ERY974 |
| GUCY2C | PF-07062119 |
| Heparanase | An antibody selected from HP130, HP 239, HP 108.264, |
| | HP 115.140, HP 152.197, HP 110.662, HP 144.141, HP |
| | 108.371, HP 135.108, HP 151.316, HP 117.372, HP |
| | 37/33, HP3/17, HP 201 or HP 102 or an amino acid |
| | sequence of SEQ ID NO: 1-11 described in US Patent |
| | Pub. US 2004/0170631. |
| Her2 | Herceptin ™ (trastuzumab) |
| Her2 | Aldesleukin (proleukine) |
| Her2 | Sargramustim (Leucine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446,175. |
| | VL: SEQ ID NO: 2 of U.S. Pat. No. 7,446,175 |
| IL12/IFN3 | Stelara ™ (ustekinumab) |
| IL1Ra | Antril ™, Kineret ™ (ankinra) |
| IL2R | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| IL12/IL23 p40 subunit | Stelara ™ (ustekinumab) |
| | VH: SEQ ID NO: 7 of U.S. Pat. No. 6,902,734 |
| | VL: SEQ ID NO: 8 of U.S. Pat. No. 6,902,734 |
| Integrin α4 | Tysabri ™ (natalizumab) |
| | VH: SEQ ID NOs: 11-13 of U.S. Pat. No. 5,840,299 |
| | VL: SEQ ID NOs: 7-8 of U.S. Pat. No. 5,840,299 |
| Integrin α4 β7 | Entyvio ™ (vedolizumab) |
| | HC: SEQ ID NO: 2 of US Patent Pub. US 2012/0282249. |
| | LC: SEQ ID NO: 4 of US Patent Pub. US 2012/0282249. |
| Integrin α5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US |
| | Patent Pub. US 2022/0089744. |
| | VL: SEQ ID NO: 4, 16, 18, 20, 22, 44-57 or 107-116 of US |
| | Patent Pub. US 2022/0089744. |
| KIR | Anti-KIR antibodies having a VH of SEQ ID NO: 5 and a |
| | VL of SEQ ID NO: 3 of PCT Pub. WO2014/066532A1 |
| KIR | Anti-KIR antibodies having a VH of SEQ ID NO: 1 and a |
| | VL of SEQ ID NO: 2 of PCT Pub. WO2012/160448A2 |
| | Anti-KIR antibodies having a VH of SEQ ID NO: 3 and a |
| | VL of SEQ ID NO: 4 of PCT Pub. WO2012/160448A2 |
| LAG3 | Relatlimab (BMS-98016) |
| LAG3 | Sym022 |
| LAG3 | HLX26 |
| LAG3 | TSR-033 |
| LAG3 | ABL501 |
| LAG3 | INCAGN02385 |
| LAG3 | Fianlimab (REGN3767) |
| LAG3 | RO7247669 |
| LAG3 | EMB-02 |
| LAG3 | FS118 |
| LAG3 | GSK2831781 |
| LAG3 | IBI323 |
| LAG3 | IBI110 |
| LAG3 | LAG525 |
| LAG3 | XmAb ®22841 |
| LAG3 | LBL-007 |
| LAG3 | VH: SEQ ID NO: 1, 8, 10 or 12 of U.S. Pat. No. 9,902,772. |
| | VL: SEQ ID NO: 2, 3, 4, 5, 6, 7, 9, 11, 13 or 14 of U.S. Pat. |
| | No. 9,902,772. |
| LAG3 | VH: SEQ ID NO: 182 of US Patent Pub. US 2021/0095026. |
| | VL: SEQ ID NO: 88 of US Patent Pub. US 2021/0095026. |
| LAG3 | Antibodies having VH/VL amino acid sequences of SEQ ID |
| | Nos 23/24, 3/4 and 11/12 of US Pub. US2022/0056126A1. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences |
| | having the amino acid sequences of any one of the |
| | following SEQ ID NO: pairs from US 2018/0118848A1: |
| | 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274,160. |
| | VL: SEQ ID NO: 2 of U.S. Pat. No. 11,274,160. |
| NGF | (tanezumab) |
| NKH1A | The monoclonal antibody deposited with ATCC and |
| | assigned accession no. HB8564, as decribed in U.S. |
| | Pat. No. 4,772,552A |
| NKP46 | Anti-NKP46 antibodies having CDR-H1, CDR-H2 and |
| | CDR-H3 sequences of SEQ ID Nos: 4, 6 and 8 and CDR- |
| | L1, CDR-L2 and CDR-L3 sequences of SEQ ID Nos: 12, |
| | 14 and 16 of PCT Pub. WO2018/047154A1 |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106/BMS-936558 (nivolumab), a human IgG4 mAb |
| | with the structure described in WHO Drug Information, Vol. |
| | 27, No. 1, pages 68-69 (2013) and whose heavy and light |
| | chain sequences are disclosed in FIG. 7 of US Pub. No. |
| | US20190270812A1 |
| | HC: SEQ ID NO: 23 of US Pub. No. US20190270812A1 |
| | LC: SEQ ID NO: 24 of US Pub. No. US20190270812A1 |
| PD1 | MK-3475 (pembrolizumab), a humanized IgG4 mAb with the |
| | structure described in WHO Drug Information, Vol. 27, No. 2, |
| | pages 161-162 (2013) and whose heavy and light chain |
| | sequences are disclosed in FIG. 6 of US Pub. No. |
| | US20190270812A1 |
| | HC: SEQ ID NO: 21 of US Pub. No. US20190270812A1 |
| | LC: SEQ ID NO: 22 of US Pub. No. US20190270812A1 |
| PD1 | REGN2810 (disclosed as H4H7798N in US Pub No. |
| | 20150203579) |
| | HC: SEQ ID NO: 330 of US Pub. No. 20150203579 |
| | LC: SEQ ID NO: 331 of US Pub. No. 20150203579 |
| PD1 | Anti-PD1 antibodies having CDR H1-H3 and CDR L1-L3 |
| | sequences corresponding to the following SEQ ID Nos. of |
| | U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; and |
| | h) SEQ ID Nos: 60, 61, 62, 63, 64, and 65, respectively; |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID NOs: 2/10, |
| | 18/26, 34/42, 50/58, 66/74, 82/90, 98/106, 1 14/122, |
| | 130/138, 146/154, 162/170, 178/186, 194/202, 210/202, |
| | 218/202, 226/202, 234/202, 242/202, 250/202, 258/202, |
| | 266/202, 274/202, 282/202, 290/202, 298/186, 306/186 and |
| | 314/186 of PCT Pub. WO2015112800A1. |
| PD1 | Anti-PD1 antibodies disclosed in U.S. Pat. No. 10,294,299 |
| | B2 as having the following SEQ ID NO. pairs for heavy and |
| | light chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb whose |
| | heavy and light chain sequences are disclosed as BAP049- |
| | Clone-E in U.S. Pat. No: 9683048 B2. |
| | HC: SEQ ID NO: 91 of U.S. Pat. No.: 9,683,048 |
| | LC: SEQ ID NO: 72 of U.S. Pat. No.: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. 7,488,802 |
| | as having the following SEQ ID NO. pairs for heavy and light |
| | chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 7,521,051 |
| | as having the following SEQ ID NO. pairs for heavy and light |
| | chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 8,008,449 |
| | as having the following SEQ ID NO. pairs for heavy and light |
| | chain variable domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. 8,354,509 |
| | as having the following SEQ ID NO. pairs for heavy and light |
| | chain variable domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. 8,168,757 |
| | as having the following SEQ ID NO. pairs for heavy and light |
| | chain variable domains: |
| | SEQ ID Nos. 4/5 |
| | SEQ ID Nos. 12/13 |
| | SEQ ID Nos. 18/19 |
| | SEQ ID Nos. 40/41 |
| | SEQ ID Nos. 47/48 |
| | SEQ ID Nos. 26/27 |
| | SEQ ID Nos. 34/35 |
| | SEQ ID Nos. 55/56 |
| | SEQ ID Nos. 67/68 |
| PD1 | Anti-PD1 antibodies described in PCT Pub. No. |
| | WO2004/004771 |
| PD1 | Anti-PD1 antibodies described in PCT Pub. No. |
| | W02004/056875 as having the following SEQ ID NO. pairs |
| | for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. |
| | WO2004/072286 |
| PD1 | VH: SEQ ID NO: 25, 26, 27, 28, or 29 of US Pub. No. |
| | US2011/0271358 |
| | VL: SEQ ID NO: 30, 31, 32, or 33 of US Pub. No. |
| | US2011/0271358 |
| PD1 | SHR-1210 (Camrelizumab) described in PCT Publication |
| | No: WO 2015/085847 as having the following heavy and |
| | light chain variable domains: |
| | HC: SEQ ID NO: 9 |
| | LC: SEQ ID NO: 10 |
| PDL1 | Durvalumab (MEDI4736) |
| | HC: SEQ ID NO: 26 of PCT application No. |
| | WO2020225552 |
| | LC: SEQ ID NO: 27 of PCT application No. |
| | WO2020225552 |
| PDL1 | Atezolizumab (Tecentriq, MPDL3280A, RG7446) |
| | HC: SEQ ID NO: 20 of U.S. Pat. No. 8,217,149 |
| | LC: SEQ ID NO: 21 of U.S. Pat. No. 8,217,149 |
| PDL1 | MDX 1105 (BMS-936559) |
| PDL1 | Anti-PD1 antibodies described in U.S. Pat. No. |
| | 7,943,743 as having the following SEQ ID NO. pairs for |
| | heavy and light chain variable domains: |
| | SEQ ID Nos. 1/11 |
| | SEQ ID Nos. 2/12 |
| | SEQ ID Nos. 3/13 |
| | SEQ ID Nos. 4/14 |
| | SEQ ID Nos. 5/15 |
| | SEQ ID Nos. 6/16 |
| | SEQ ID Nos. 7/17 |
| | SEQ ID Nos. 8/18 |
| | SEQ ID Nos. 9/19 |
| | SEQ ID Nos. 10/20 |
| PDL1 | Avelumab, described in U.S. Pat. No.: 9,624,298 as |
| | having the following heavy and light chain variable domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID |
| | Nos. 52, 56, and 77 of PCT Pub. No. 2018/080812 |
| | CDR-L1, CDR-L2 and CDR-L3 sequences of SEQ ID Nos: |
| | 65, 42, and 71 of PCT Pub. No. 2018/080812 |
| PDL1 | KN035 (Envafolimab) is a nanobody described as Hu56V2 |
| | in U.S. Pat. No. 11,225,522 as having the VHH SEQ ID |
| | NO: 34 |
| PDL1 | LY3434172 |
| PDL1 | LY3300054 (lodapolimab) described in PCT Pub No: WO |
| | 2017/034916 as having the following heavy and light chain |
| | variable domains: |
| | HC: SEQ ID NO: 10 |
| | L: SEQ ID NO: 11 |
| PDL1 | LDP (lesabelimab, ADG104) described in CN Patent No: |
| | 114225023 as having the following heavy and light chain |
| | variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No. |
| | 10,118,963 as having the following heavy and light chain |
| | variable domains: |
| | HC: SEQ ID NO: 1 |
| | LC: SEQ ID NO: 2 |
| PDL1 | BGB-A333 (garivulimab) described in U.S. Pat. No. |
| | 11,512,132 as having the following heavy and light chain |
| | variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. |
| | 11,168,144. |
| | VL: SEQ ID NO: 58, 137 or 12 of U.S. Pat. No. |
| | 11,168,144. |
| PDL1 | VH: SEQ ID NO: 23, 124, 126, 127, 128, 130, 140 or 145 |
| | of U.S. Pat. No. 11,208,486. |
| | VL: SEQ ID NO: 24 or 125 of U.S. Pat. No. 11,208,486. |
| Phosphatidylserine | (bavituximab) |
| PSCA | GEM3PSCA |
| PSMA | huJ591 |
| PSMA | Anti-PSMA antibodies having VH and VL sequences |
| | having the amino acid sequences of any one of the |
| | following SEQ ID NO: pairs from WO 2017/023761A1: |
| | 2/1642; 10/1642; 18/1642; 26/1642; 34/1642; 42/1642; |
| | 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, |
| | PSMA 3.11, PSMA 5.4, PSMA 7.1, PSMA 7.3, PSMA |
| | 10.3, PSMA 1.8.3, PSMA A3.1.3, PSMA A3.3.1, Abgenix |
| | 4.248.2, Abgenix 4.360.3, Abgenix 4.7.1, Abgenix 4.4.1, |
| | Abgenix 4.177.3, Abgenix 4.16.1, Abgenix 4.22.3, |
| | Abgenix 4.28.3, Abgenix 4.40.2, Abgenix 4.48.3, Abgenix |
| | 4.49.1, Abgenix 4.209.3, Abgemx 4.219.3, Abgenix |
| | 4.288.1, Abgenix 4.333.1, Abgemx 4.54.1, Abgenix |
| | 4.153.1, Abgenix 4.232.3, Abgenix 4.292.3, Abgenix |
| | 4.304.1, Abgenix 4.78.1 and Abgenix 4.152.1 described in |
| | WO2003034903A2 |
| | A hybridoma cell line such as: PSMA 3.7 (PTA-3257), |
| | PSMA 3.8, PSMA 3.9 (PTA- 3258), PSMA 3.11 (PTA- |
| | 3269), PSMA 5.4 (PTA-3268), PSMA 7.1 (PTA-3292), |
| | PSMA 7.3 (PTA-3293), PSMA 10.3 (PTA-3247) , PSMA |
| | 1.8.3 (PTA-3906), PSMA A3.1.3 (PTA- 3904), PSMA |
| | A3.3.1 (PTA-3905), Abgenix 4.248.2 (PTA-4427), Abgenix |
| | 4.360.3 (PTA- 4428), Abgenix 4.7.1 (PTA-4429), Abgenix |
| | 4.4.1 (PTA-4556), Abgenix 4.177.3 (PTA-4557), Abgenix |
| | 4.16.1 (PTA-4357), Abgenix 4.22.3 (PTA-4358), Abgenix |
| | 4.28.3 (PTA-4359), Abgenix 4.40.2 (PTA-4360), Abgenix |
| | 4.48.3 (PTA-4361), Abgenix 4.49.1 (PTA-4362), Abgenix |
| | 4.209.3 (PTA-4365), Abgenix 4.219.3 (PTA-4366), |
| | Abgenix 4.288.1 (PTA-4367), Abgenix 4.333.1 (PTA- |
| | 4368), Abgenix 4.54.1 (PTA-4363), Abgenix 4.153.1 |
| | (PTA-4388), Abgenix 4.232.3 (PTA-4389), Abgenix |
| | 4.292.3 (PTA-4390), Abgenix 4.304.1 (PTA-4391), |
| | Abgenix 4.78.1 (PTA-4652), and Abgemx 4.152.1(PTA- |
| | 4653) described in WO 2003/034903A2. |
| | VH of SEQ ID Nos: 2-7 described in WO 2003/034903A2 |
| | VL of SEQ ID Nos: 8-13 described in WO 2003/034903A2 |
| PMSA | VH: SEQ ID Nos: 225, 239, 253, 267, 281, 295, 309, 323, |
| | 337, 351, 365, 379, 393, 407, 421, 435, 449, 463, 477, |
| | 491, 505, 519, 533, 547, 561, 575, 589, 603 or 617 |
| | described in WO 2011/121110A1. |
| | VL SEQ ID Nos: 230, 244, 258, 272, 286, 300, 314, 328, |
| | 342, 356, 370, 384, 398, 412, 426, 440, 454, 468, 482, |
| | 496, 510, 524, 538, 552, 566, 580, 594, 608 or 622 |
| | described in WO 2011/121110A1. |
| | VH and VL SEQ ID Nos: 235, 249, 263, 277, 291, 305, |
| | 319, 333, 347, 361, 375, 389, 403, 417, 431, 445, 459, |
| | 473, 487, 501, 515, 529, 543, 557, 571, 585, 599, 613 or |
| | 627 described in WO 2011/121110A1. |
| PMSA | An anti-PMSA antibody having a VL amino acid sequence |
| | of any one of SEQ ID Nos: 229-312 of US 2022/0119525 |
| | A1 and a VH of SEQ ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of US Patent Pub. 2017/0002060 |
| | VL: SEQ ID NO: 52 of US Patent Pub. 2017/0002060 |
| SLAMF7 | Empliciti ™ (elotuzumab) |
| SSTR2 | XmAb ®18087 |
| STEAP1 | VHCDR1 SEQ ID Nos: 14, 33, 182, 184 or 185 described |
| | in US20210179731A1. |
| | VHCDR2 SEQ ID Nos: 15, 21, 34, 182, 184 or 185 |
| | described in US20210179731A1. |
| | VHCDR3 SEQ ID Nos: 16 and 35 described in |
| | US20210179731A1. |
| | VH SEQ ID Nos: 182 or 184 described in |
| | US20210179731A1. |
| | VLCDR1 SEQ ID Nos: 11 or 30 described in |
| | US20210179731A1. |
| | VLCDR2 SEQ ID Nos: 12 or 31 described in |
| | US20210179731A1. |
| | VLCDR3 SEQ ID Nos: 13 or 32 described in |
| | US20210179731A1. |
| | VL SEQ ID Nos: 183 or 186 described in |
| | US20210179731A1. |
| STEAP1 | AMG509 |
| STEAP2 | Anti-STEAP 2 antibodies having CDR-H1, CDR-H2, CDR- |
| | H3, CDR-L1, CDR-L2 and CDR-L3 sequences selected |
| | from SEQ ID NOS: (1) 4-6-8-12-14-16; (2) 20-22-24-28- |
| | 30-32; (3) 36-38-40-44-46-48; (4) 52-54-56-60-62-64; (5) |
| | 68-70-72-60-62-64; (6) 76-78-80-60-62-64; (7) 84-86-88- |
| | 60-62-64; (8) 92-94-96-60-62-64; (9) 100-102-104-60-62- |
| | 64; (10) 108-110-112-116-118-120; (11) 124-126-128- |
| | 132-134-136; (12) 140-142-144-148-150-152; (13) 156- |
| | 158-160-164-166-168; (14) 172-174-176-180-182-184; |
| | (15) 188-190-192-196-198-200; (16) 204-206-208-212- |
| | 214-216; (17) 220-222-224-228-230-232; (18) 236-238- |
| | 240-244-246-248; (19) 252-254-256-260-262-264; (20) |
| | 268-270-272-276-278-280; (21) 284-286-288-292-294- |
| | 296; (22) 300-302-304-308-310-312; (23) 316-318-320- |
| | 324-326-328; (24) 332-334-336-340-342-344; (25) 348- |
| | 350-352-356-358-360; (26) 364-366-368-372-374-376; |
| | and (27) 380-382-384-388-390-392 of U.S. Pat. No. |
| | 10,772,972 B2. |
| | Anti-STEAP 2 antibodies having (a) a VH comprising the |
| | amino acid of any one of SEQ ID Nos: 2, 18, 34, 50, 66, |
| | 74, 82, 90, 98, 106, 122, 138, 154, 170, 186, 202, 218, |
| | 234, 250, 266, 282, 298, 314, 330, 346, 362, and 378 of |
| | U.S. Pat. No. 10,772,972 B2; and (b) a VL comprising |
| | the amino acid sequence of any one of SEQ ID NOs: 10; |
| | 26; 42; 58; 114; 130; 146; 162; 178; 194; 210; 226, 242; |
| | 258; 274; 290; 306; 322; 338; 354; 370; and 386 of U.S. |
| | Pat. No. 10,772,972 B2. |
| | Anti-STEAP 2 antibodies having a VH/VL pair comprising |
| | the amino acid sequences of any of the following pairs of |
| | SEQ ID Nos of U.S. Pat. No. 10,772,972 B2: 2/10; 18/26; |
| | 34/42; 50/58; 66/58; 74/58; 82/58; 90/58; 98/58; 106/114; |
| | 122/130; 138/146; 154/162; 170/178; 186/194; 202/210; |
| | 218/226; 234/242; 250/258; 266/274; 282/290; 298/306; |
| | 314/322; 330/338; 346/354; 362/370; and 378/386. |
| Syndecan-1 (CD 138) | The B-B4 antibody described in Wijdenes et al. (1996) Br. |
| | J. Haematol., 94: 318-323 |
| Syndecan-4 | The amino acid sequence of amino acids 93 and 121 of |
| | SEQ ID NO: 1 or the amino acid sequence of amino acids |
| | 92 and 122 of SEQ ID NO: 2 described in European Patent |
| | Pub. EP 2 603 236. |
| TGFβ | GC1008 |
| TNFR | Enbrel ™ (etanercept) |
| TNFα | Remicade ™ (infliximab) |
| | VH: SEQ ID NO: 2 of Int. Patent Publication |
| | WO201/3087911 A1 |
| | VH: SEQ ID NO: 3 of Int. Patent Publication WO2013/ |
| | A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |
| XCR1 | Anti-XCR1 antibodies disclosed in U.S. Pat. No. 9,371,389 |
| | B2, including: |
| | The antibodies designated 2H6, 5G7, 11H2, |
| | HK1L2 and HK5L5 |
| | Antibodies having CDR-H1, CDR-H2 and CDR-H3 |
| | sequences of SEQ ID Nos: 53-55 and CDR-L1, |
| | CDR-L2 and CDR-L3 sequences of SEQ ID |
| | Nos: 56-58. |
| | Antibodies having CDR-H1, CDR-H2 and CDR-H3 |
| | sequences of SEQ ID Nos: 41-43 and CDR-L1, |
| | CDR-L2 and CDR-L3 sequences of SEQ ID |
| | Nos: 44-46. |

In some aspects, the targeting moiety competes with an antibody set forth in Table F for binding to the target molecule. In further aspects, the targeting moiety comprises CDRs having CDR sequences of an antibody set forth in Table F. In some embodiments, the targeting moiety comprises all 6 CDR sequences of the antibody set forth in Table F. In other embodiments, the targeting moiety comprises at least the heavy chain CDR sequences (CDR-H1, CDR-H2, CDR-H3) of an antibody set forth in Table F and the light chain CDR sequences of a universal light chain. In further aspects, a targeting moiety comprises a VH comprising the amino acid sequence of the VH of an antibody set forth in Table F. In some embodiments, the targeting moiety further comprises a VL comprising the amino acid sequence of the VL of the antibody set forth in Table F. In other embodiments, the targeting moiety further comprises a universal light chain VL sequence.

In some embodiments, the target molecule is PDL1. Table F-1 below provides exemplary anti-PDL1 antibodies and/or antibody sequences upon which the targeting moiety can be based, e.g., which can be incorporated into a targeting moiety for use in the interferon proproteins of the disclosure.

TABLE F-1
Exemplary PDL1 antibodies and/or binding sequences

| Target | Antibody Name and/or Binding Sequences |
|---|---|
| PDL1 | Durvalumab (MEDI4736) |
| | HC: SEQ ID NO: 26 of PCT application No. WO2020225552 |
| | LC: SEQ ID NO: 27 of PCT application No. WO2020225552 |
| PDL1 | Atezolizumab (Tecentriq, MPDL3280A, RG7446) |
| | HC: SEQ ID NO: 20 of U.S. Pat. No. 8,217,149 |
| | LC: SEQ ID NO: 21 of U.S. Pat. No. 8,217,149 |
| PDL1 | MDX 1105 (BMS-936559) |
| PDL1 | Anti-PDL1 antibodies described in U.S. Pat. No. |
| | 7,943,743 as having the following SEQ ID NO. |
| | pairs for heavy and light chain variable domains: |
| | SEQ ID Nos: 1/11 |
| | SEQ ID Nos: 2/12 |
| | SEQ ID Nos: 3/13 |
| | SEQ ID Nos: 4/14 |
| | SEQ ID Nos: 5/15 |
| | SEQ ID Nos: 6/16 |
| | SEQ ID Nos: 7/17 |
| | SEQ ID Nos: 8/18 |
| | SEQ ID Nos: 9/19 |
| | SEQ ID Nos: 10/20 |
| PDL1 | Avelumab, described in U.S. Pat. No.: 9,624,298 as having |
| | the following heavy and light chain variable domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID Nos: |
| | 52, 56, and 77 of PCT Pub. No. 2018/080812 |
| | CDR-L1, CDR-L2 and CDR-L3 sequences of SEQ ID Nos: |
| | 65, 42, and 71 of PCT Pub. No. 2018/080812 |
| PDL1 | KN035 (Envafolimab) is a nanobody described as |
| | Hu56V2 in U.S. Pat. No. 11,225,522 as having the |
| | VHH SEQ ID NO: 34 |
| PDL1 | LY3434172 |
| PDL1 | LY3300054 (lodapolimab) described in PCT Pub No: |
| | WO 2017/034916 as having the following heavy and |
| | light chain variable domains: |
| | HC: SEQ ID NO: 10 |
| | L: SEQ ID NO: 11 |
| PDL1 | LDP (lesabelimab, ADG104) described in CN Patent |
| | No: 114225023 as having the following heavy and |
| | light chain variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No.: |
| | 10,118,963 as having the following heavy and |
| | light chain variable domains: |
| | HC: SEQ ID NO: 1 |
| | LC: SEQ ID NO: 2 |
| PDL1 | BGB-A333 (garivulimab) described in U.S. Pat. No.: |
| | 11,512,132 as having the following heavy and |
| | light chain variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or |
| | 145 of U.S. Pat. No. 11,208,486. |
| | VL: SEQ ID NO: 24 or 125 of U.S. Pat. No. 11,208,486. |

In some aspects, the targeting moiety competes with an anti-PDL1 antibody set forth in Table F-1 for binding to PDL1. In further aspects, the targeting moiety comprises CDRs having CDR sequences of an anti-PDL1 antibody set forth in Table F-1. In some embodiments, the targeting moiety comprises all 6 CDR sequences of the anti-PDL1 antibody set forth in Table F-1. In other embodiments, the targeting moiety comprises at least the heavy chain CDR sequences (CDR-H1, CDR-H2, CDR-H3) of an anti-PDL1 antibody set forth in Table F-1 and the light chain CDR sequences of a universal light chain. In further aspects, a targeting moiety comprises a VH comprising the amino acid sequence of the VH of an anti-PDL1 antibody set forth in Table F-1. In some embodiments, the targeting moiety further comprises a VL comprising the amino acid sequence of the VL of the anti-PDL1 antibody set forth in Table F-1. In other embodiments, the targeting moiety further comprises a universal light chain VL sequence.

In some embodiments, the target molecule is PD1. Table F-2 below provides exemplary anti-PD1 antibodies and/or antibody sequences upon which the targeting moiety can be based, e.g., which can be incorporated into a targeting moiety for use in the interferon proproteins of the disclosure.

TABLE F-2
Exemplary PD1 antibodies and/or binding sequences

| Target | Antibody Name and/or Binding Sequences |
|---|---|
| PD1 | MDX-1106/BMS-936558 (nivolumab), a human IgG4 |
| | mAb with the structure described in WHO Drug |
| | Information, Vol. 27, No. 1, pages 68-69 (2013) |
| | and whose heavy and light chain sequences are |
| | disclosed in FIG. 7 of US Pub. No. US20190270812A1 |
| | HC: SEQ ID NO: 23 of US Pub. No. US20190270812A1 |
| | LC: SEQ ID NO: 24 of US Pub. No. US20190270812A1 |
| PD1 | MK-3475 (pembrolizumab), a humanized IgG4 mAb with |
| | the structure described in WHO Drug Information, Vol. |
| | 27, No. 2, pages 161-162 (2013) and whose heavy and |
| | light chain sequences are disclosed in FIG. 6 of |
| | US Pub. No. US20190270812A1 |
| | HC: SEQ ID NO: 21 of US Pub. No. US20190270812A1 |
| | LC: SEQ ID NO: 22 of US Pub. No. US20190270812A1 |
| PD1 | H4H7798N as disclosed in US Pub No. 20150203579 |
| | HC: SEQ ID NO: 330 of US Pub. No. 20150203579 |
| | LC: SEQ ID NO: 331 of US Pub. No. 20150203579 |
| PD1 | Anti-PD1 antibodies having CDR H1-H3 and CDR L1-L3 |
| | sequences corresponding to the following SEQ ID Nos. |
| | of U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; and |
| | h) SEQ ID Nos: 60, 61, 62, 63, 64, and 65, respectively. |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID Nos: 2/10, |
| | 18/26, 34/42, 50/58, 66/74, 82/90, 98/106, 1 14/122, |
| | 130/138, 146/154, 162/170, 178/186, 194/202, 210/202, |
| | 218/202, 226/202, 234/202, 242/202, 250/202, 258/202, |
| | 266/202, 274/202, 282/202, 290/202, 298/186, 306/186 |
| | and 314/186 of PCT Pub. WO2015112800A1. |
| PD1 | Anti-PD1 antibodies disclosed in U.S. Pat. No. |
| | 10,294,299 B2 as having the following SEQ ID NO. |
| | pairs for heavy and light chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb |
| | whose heavy and light chain sequences are disclosed |
| | as BAP049-Clone-E in U.S. Pat. No.: 9683048 B2. |
| | HC: SEQ ID NO: 91 of U.S. Pat. No.: 9,683,048 |
| | LC: SEQ ID NO: 72 of U.S. Pat. No.: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. 7,488,802 |
| | as having the following SEQ ID NO. pairs for heavy and |
| | light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 7,521,051 |
| | as having the following SEQ ID NO. pairs for heavy and |
| | light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 8,008,449 |
| | as having the following SEQ ID NO. pairs for heavy and |
| | light chain variable domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. 8,354,509 |
| | as having the following SEQ ID NO. pairs for heavy and |
| | light chain full-length domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. 8,168,757 |
| | as having the following SEQ ID NO. pairs for heavy and |
| | light chain variable domains: |
| | SEQ ID Nos. 4/5 |
| | SEQ ID Nos. 12/13 |
| | SEQ ID Nos. 18/19 |
| | SEQ ID Nos.40/41 |
| | SEQ ID Nos.47/48 |
| | SEQ ID Nos.26/27 |
| | SEQ ID Nos.34/35 |
| | SEQ ID Nos.55/56 |
| | SEQ ID Nos.67/68 |
| PD1 | Anti-PD1 antibodies described in PCT Pub. No. |
| | WO2004/004771 |
| PD1 | Anti-PD1 antibodies described in PCT Pub. No. |
| | WO2004/056875 as having the following SEQ ID NO. |
| | pairs for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. |
| | WO2004/072286 |
| PD1 | VH: SEQ ID NO: 25, 26, 27, 28, or 29 of US Pub. No. |
| | US2011/0271358 |
| | VL: SEQ ID NO: 30, 31, 32, or 33 of US Pub. No. |
| | US2011/0271358 |
| PD1 | SHR-1210 (Camrelizumab) described in PCT |
| | Publication No: WO |
| | 2015/085847 as having the following heavy and light |
| | chain variable domains: |
| | HC: SEQ ID NO: 9 |
| | LC: SEQ ID NO: 10 |

In some aspects, the targeting moiety competes with an anti-PD1 antibody set forth in Table F-2 for binding to PD1. In further aspects, the targeting moiety comprises CDRs having CDR sequences of an anti-PD1 antibody set forth in Table F-2. In some embodiments, the targeting moiety comprises all 6 CDR sequences of the anti-PD1 antibody set forth in Table F-2. In other embodiments, the targeting moiety comprises at least the heavy chain CDR sequences (CDR-H1, CDR-H2, CDR-H3) of an anti-PD1 antibody set forth in Table F-2 and the light chain CDR sequences of a universal light chain. In further aspects, a targeting moiety comprises a VH comprising the amino acid sequence of the VH of an anti-PD1 antibody set forth in Table F-2. In some embodiments, the targeting moiety further comprises a VL comprising the amino acid sequence of the VL of the anti-PD1 antibody set forth in Table F-2. In other embodiments, the targeting moiety further comprises a universal light chain VL sequence.

Where the target molecule is a checkpoint inhibitor, in some embodiments, the checkpoint inhibitor targeting moiety is non-blocking or poorly-blocking of ligand-receptor binding. Examples of non-blocking or poorly-blocking anti-PD1 antibodies includes antibodies having VH/VL amino acid sequences of SEQ ID Nos: 2/10 of PCT Pub. No. WO2015/112800A1; SEQ ID Nos: 16/17 of U.S. Pat. No. 11,034,765 B2; SEQ ID Nos. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID Nos 23/24, 3/4 and 11/12 of US Pub. US2022/0056126A1.

Additional target molecules that can be targeted by the IFN proproteins are disclosed in Table I below and in, e.g., Hafeez et al., 2020, Molecules 25:4764, doi: 10.3390/molecules25204764, particularly in Table 1. Table 1 of Hafeez et al. is incorporated by reference in its entirety here.

### 6.7. Targeting Moiety Formats

In certain aspects, the targeting moiety of an IFN proprotein of the disclosure can be any type of antibody or fragment thereof that retains specific binding to an antigenic determinant. In one embodiment the targeting moiety is an immunoglobulin molecule or fragment thereof, particularly an IgG class immunoglobulin molecule, more particularly an IgG1 or IgG4 immunoglobulin molecule. Antibody fragments include, but are not limited to, VH (or VA) fragments, VL (or VL) fragments, Fab fragments, F(ab′) 2 fragments, scFv fragments, Fv fragments, minibodies, diabodies, triabodies, and tetrabodies.

### 6.7.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain. When the targeting moiety is a Fab, the CH1 of the Fab may represent the constant domain N-terminal to the IFN moiety, for example as shown in FIGS. 1B and 1C.

For the IFN proproteins of the disclosure, particularly when the light chains of the targeting moieties are not common or universal light chains, it is advantageous to use Fab heterodimerization strategies to permit the correct association of Fab domains belonging to the same targeting moiety and minimize aberrant pairing of Fab domains belonging to different targeting moieties. For example, the Fab heterodimerization strategies shown in Table G below can be used:

TABLE G
Fab Heterodimerization Strategies

| STRATEGY | VH | CH1 | VL | CL | REFERENCE |
|---|---|---|---|---|---|
| CrossMabCH1-CL | WT | CL domain | WT | CH1 domain | Schaefer et al., 2011, |
| | | | | | Cancer Cell 2011; |
| | | | | | 20: 472-86; |
| | | | | | PMID: 22014573. |
| orthogonal Fab | 39K, | H172A, | 1R, 38D, | L135Y, | Lewis et al., 2014, Nat |
| VHVRD1CH1CRD2 - | 62E | F174G | (36F) | S176W | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs |
| | | | | | 7: 364-76 |
| CR3 | WT | T192E | WT | N137K, | Golay at al., 2016, J |
| | | | | S114A | Immunol 196: 3199-211. |
| MUT4 | WT | L143Q, | WT | V133T, | Golay at al., 2016, J |
| | | S188V | | S176V | Immunol 196: 3199-211. |
| DuetMab | WT | F126C | WT | S121C | Mazor et al., 2015, MAbs |
| | | | | | 7: 377-89; Mazor et al., |
| | | | | | 2015, MAbs 7: 461-669. |
| Domain | WT | CH3 + knob or | WT | CH3 + hole or | Wozniak-Knopp et al., |
| exchanged | | hole mutation | | knob mutation | 2018, |
| | | | | | PLoSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH: VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, Mabs 7:377-89).

Fab domains can also be modified by replacing the CH1 domain and CL domain with alternative domains that promote correct assembly. For example, Wu et al., 2015, Mabs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each unique ABD in the IFN proproteins of the disclosure. In various embodiments, employing a common light chain as described herein reduces the number of inappropriate species in the IFN proproteins as compared to employing original cognate VLs. In various embodiments, the VL domains of ABDs are identified from monospecific antibodies comprising a common light chain. In various embodiments, the VH regions of the ABDs in the IFN proproteins comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

### 6.7.2. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the non-cleavable linkers identified in Section 6.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5 (typically a repeat of a sequence containing the amino acids glycine and serine, such as the amino acid sequence (Gly4~Ser)3 (SEQ ID NO: 180), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.8. Fc Regions

The IFN proproteins of the disclosure typically include a pair of Fc domains that associate to form an Fc region. In native antibodies, Fc regions comprise hinge regions at their N-termini to form a constant domain. Throughout this disclosure, the reference to an Fc domain encompasses an Fc domain with a hinge domain at its N-terminus unless specified otherwise.

The Fc domains can be derived from any suitable species operably linked to an ABD or component thereof. In one embodiment the Fc domain is derived from a human Fc domain. In preferred embodiments, the targeting moiety or component thereof is fused to an IgG Fc molecule. A targeting moiety or component thereof may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4. Exemplary sequences of Fc domains from IgG1, IgG2, IgG3, and IgG4 are provided in Table Y, below.

TABLE Y
Fc Sequences

| | | SEQ ID |
|---|---|---|
| Fc | Sequence | NO |
| hIgG1 Fc | EPKSCDKTHTCPPCPAPELLGGPSVFLFPPKPKDTLMISRTPEVTC | 392 |
| (amino acids | VVVDVSHEDPEVKFNWYVDGVEVHNAKTKPREEQYNSTYRVVSV | |
| 99-330 of | LTVLHQDWLNGKEYKCKVSNKALPAPIEKTISKAKGQPREPQVYTL | |
| UniprotKB | PPSRDELTKNQVSLTCLVKGFYPSDIAVEWESNGQPENNYKTTPP | |
| P01857-1) | VLDSDGSFFLYSKLTVDKSRWQQGNVFSCSVMHEALHNHYTQKS | |
| | LSLSPGK | |
| hIgG2 Fc | ERKCCVECPPCPAPPVAGPSVFLFPPKPKDTLMISRTPEVTCVVV | 393 |
| (amino acids | DVSHEDPEVQFNWYVDGVEVHNAKTKPREEQFNSTFRVVSVLTV | |
| 99-326 of | VHQDWLNGKEYKCKVSNKGLPAPIEKTISKTKGQPREPQVYTLPP | |
| UniprotKB | SREEMTKNQVSLTCLVKGFYPSDISVEWESNGQPENNYKTTPPML | |
| P01859-1) | DSDGSFFLYSKLTVDKSRWQQGNVFSCSVMHEALHNHYTQKSLS | |
| | LSPGK | |
| hIgG3 Fc | ELKTPLGDTTHTCPRCPEPKSCDTPPPCPRCPEPKSCDTPPPCPR | 394 |
| (amino acids | CPEPKSCDTPPPCPRCPAPELLGGPSVFLFPPKPKDTLMISRTPEV | |
| 99-377 of | TCVVVDVSHEDPEVQFKWYVDGVEVHNAKTKPREEQYNSTFRVV | |
| UniprotKB | SVLTVLHQDWLNGKEYKCKVSNKALPAPIEKTISKTKGQPREPQVY | |
| P01860-1) | TLPPSREEMTKNQVSLTCLVKGFYPSDIAVEWESSGQPENNYNTT | |
| | PPMLDSDGSFFLYSKLTVDKSRWQQGNIFSCSVMHEALHNRFTQ | |
| | KSLSLSPGK | |
| hIgG4 Fc | ESKYGPPCPSCPAPEFLGGPSVFLFPPKPKDTLMISRTPEVTCVVV | 395 |
| (amino acids | DVSQEDPEVQFNWYVDGVEVHNAKTKPREEQFNSTYRVVSVLTV | |
| 99-327 of | LHQDWLNGKEYKCKVSNKGLPSSIEKTISKAKGQPREPQVYTLPP | |
| UniprotKB | SQEEMTKNQVSLTCLVKGFYPSDIAVEWESNGQPENNYKTTPPVL | |
| P01861-1) | DSDGSFFLYSRLTVDKSRWQEGNVFSCSVMHEALHNHYTQKSLS | |
| | LSLGK | |

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 410. In cases where an Fc domain comprises at least 90% sequence identity and less than 100% sequence identity to SEQ ID NO: 410 (e.g., between 90% and 99% sequence identity to SEQ ID NO: 410), an Fc domain may also comprise one or more amino acid substitutions described herein, for example one or more substitutions that reduce effector function (e.g., as described in Section 6.8.1) and/or one or more substitutions that promote Fc heterodimerization (e.g., as described in Section 6.8.2).

In some aspects, an Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO: 411. In cases where an Fc domain comprises at least 90% sequence identity and less than 100% sequence identity to SEQ ID NO: 411 (e.g., between 90% and 99% sequence identity to SEQ ID NO: 411), an Fc domain may also comprise one or more amino acid substitutions described herein, for example one or more substitutions that reduce effector function (e.g., as described in Section 6.8.1) and/or one or more substitutions that promote Fc heterodimerization (e.g., as described in Section 6.8.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 412. In cases where an Fc domain comprises at least 90% sequence identity and less than 100% sequence identity to SEQ ID NO: 412 (e.g., between 90% and 99% sequence identity to SEQ ID NO: 412), an Fc domain may also comprise one or more amino acid substitutions described herein, for example one or more substitutions that reduce effector function (e.g., as described in Section 6.8.1) and/or one or more substitutions that promote Fc heterodimerization (e.g., as described in Section 6.8.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 413. In cases where an Fc domain comprises at least 90% sequence identity and less than 100% sequence identity to SEQ ID NO: 413 (e.g., between 90% and 99% sequence identity to SEQ ID NO: 413), an Fc domain may also comprise one or more amino acid substitutions described herein, for example one or more substitutions that reduce effector function (e.g., as described in Section 6.8.1) and/or one or more substitutions that promote Fc heterodimerization (e.g., as described in Section 6.8.2).

The two Fc domains within the Fc region can be the same or different from one another. In a native antibody the Fc domains are typically identical, but for the purpose of producing multispecific binding molecules, e.g., the IFN proproteins of the disclosure and MBMs produced by their activation, the Fc domains might advantageously be different to allow for heterodimerization, as described in Section 6.8.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In IFN proproteins of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the IFN proproteins of the present disclosure may include variants of the naturally occurring constant domains described above. Such variants may comprise one or more amino acid variations compared to wild type constant domains. In one example the Fc region of the present disclosure comprises at least one constant domain that varies in sequence from the wildtype constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer and is believed to have an important role in polymerization. The tailpiece also contains a glycosylation site. In certain embodiments, the IFN proproteins of the present disclosure do not comprise a tailpiece.

The Fc domains that are incorporated into the IFN proproteins of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.8.1.

The Fc domains can also be altered to include modifications that improve manufacturability of asymmetric IFN proproteins, for example by allowing heterodimerization, which is the preferential pairing of non-identical Fc domains over identical Fc domains. Heterodimerization permits the production of IFN proproteins in which different polypeptide components are connected to one another by an Fc region containing Fc domains that differ in sequence. Examples of heterodimerization strategies are exemplified in Section 6.8.2.

It will be appreciated that any of the modifications mentioned above can be combined in any suitable manner to achieve the desired functional properties and/or combined with other modifications to alter the properties of the IFN proproteins.

### 6.8.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IFN proprotein half antibody) or the Fc region (e.g., one or both Fc domains of an IFN proprotein that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table H below. In some embodiments, the Fc domain includes only the bolded portion of the sequences shown below:

TABLE H

| | | SEQ ID |
|---|---|---|
| Fc Domain | Sequence | NO: |
| SEQ ID NO: 1 of | Asp Lys Arg Val Glu Ser Lys Tyr Gly Pro Cys Pro | 369 |
| WO2014/121087 | Pro Cys Pro Ala Pro Pro Val Ala Gly Pro Ser Val | |
| | Phe Leu Phe Pro Pro Lys Pro Lys Asp Thr Leu | |
| | Met Ile Ser Arg Thr Pro Glu Val Thr Cys Val Val | |
| | Val Asp Val Ser Gln Glu Asp Pro Glu Val Gln Phe | |
| | Asn Trp Tyr Val Asp Gly Val Glu Val His Asn Ala | |
| | Lys Thr Lys Pro Arg Glu Glu GIn Phe Asn Ser Thr | |
| | Tyr Arg Val Val Ser Val Leu Thr Val Leu His Gln | |
| | Asp Trp Leu Asn Gly Lys Glu Tyr Lys Cys Lys Val | |
| | Ser Asn Lys Gly Leu Pro Ser Ser Ile Glu Lys Thr | |
| | Ile Ser Lys Ala Lys Gly Gln Pro Arg Glu Pro Gln | |
| | Val Tyr Thr Leu Pro Pro Ser Gln Glu Glu Met Thr | |
| | Lys Asn Gln Val Ser Leu Thr Cys Leu Val Lys Gly | |
| | Phe Tyr Pro Ser Asp Ile Ala Val Glu Trp Glu Ser | |
| | Asn Gly Gln Pro Glu Asn Asn Tyr Lys Thr Thr Pro | |
| | Pro Val Leu Asp Ser Asp Gly Ser Phe Phe Leu | |
| | Tyr Ser Arg Leu Thr Val Asp Lys Ser Arg Trp Gln | |
| | Glu Gly Asn Val Phe Ser Cys Ser Val Met His Glu | |
| | Ala Leu His Asn His Tyr Thr Gln Lys Ser Leu Ser | |
| | Leu Ser Leu Gly Lys | |
| SEQ ID NO: 2 of | Asp Lys Lys Val Glu Pro Lys Ser Cys Asp Lys Thr | 370 |
| WO2014/121087 | His Thr Cys Pro Pro Cys Pro Ala Pro Pro Val Ala | |
| | Gly Pro Ser Val Phe Leu Phe Pro Pro Lys Pro Lys | |
| | Asp Thr Leu Met Ile Ser Arg Thr Pro Glu Val Thr | |
| | Cys Val Val Val Asp Val Ser Gln Glu Asp Pro Glu | |
| | Val Gln Phe Asn Trp Tyr Val Asp Gly Val Glu Val | |
| | His Asn Ala Lys Thr Lys Pro Arg Glu Glu GIn Phe | |
| | Asn Ser Thr Tyr Arg Val Val Ser Val Leu Thr Val | |
| | Leu His Gin Asp Trp Leu Asn Gly Lys Glu Tyr Lys | |
| | Cys Lys Val Ser Asn Lys Gly Leu Pro Ser Ser Ile | |
| | Glu Lys Thr Ile Ser Lys Ala Lys Gly Gln Pro Arg | |
| | Glu Pro Gln Val Tyr Thr Leu Pro Pro Ser Arg Asp | |
| | Glu Leu Thr Lys Asn Gln Val Ser Leu Thr Cys | |
| | Leu Val Lys Gly Phe Tyr Pro Ser Asp Ile Ala Val | |
| | Glu Trp Glu Ser Asn Gly Gln Pro Glu Asn Asn Tyr | |
| | Lys Thr Thr Pro Pro Val Leu Asp Ser Asp Gly Ser | |
| | Phe Phe Leu Tyr Ser Lys Leu Thr Val Asp Lys | |
| | Ser Arg Trp Gln Gln Gly Asn Val Phe Ser Cys Ser | |
| | Val Met His Glu Ala Leu His Asn His Tyr Thr Gln | |
| | Lys Ser Leu Ser Leu Ser Pro Gly Lys | |
| SEQ ID NO: 30 of | Ala Ser Thr Lys Gly Pro Ser Val Phe Pro Leu Ala | 371 |
| WO2014/121087 | Pro Ser Ser Lys Ser Thr Ser Gly Gly Thr Ala Ala | |
| | Leu Gly Cys Leu Val Lys Asp Tyr Phe Pro Glu | |
| | Pro Val Thr Val Ser Trp Asn Ser Gly Ala Leu Thr | |
| | Ser Gly Val His Thr Phe Pro Ala Val Leu Gln Ser | |
| | Ser Gly Leu Tyr Ser Leu Ser Ser Val Val Thr Val | |
| | Pro Ser Ser Ser Leu Gly Thr Gln Thr Tyr Ile Cys | |
| | Asn Val Asn His Lys Pro Ser Asn Thr Lys Val Asp | |
| | Lys Lys Val Glu Pro Lys Ser Cys Asp Lys Thr | |
| | His Thr Cys Pro Pro Cys Pro Ala Pro Pro Val | |
| | Ala Gly Pro Ser Val Phe Leu Phe Pro Pro Lys | |
| | Pro Lys Asp Thr Leu Met Ile Ser Arg Thr Pro | |
| | Glu Val Thr Cys Val Val Val Asp Val Ser Gln | |
| | Glu Asp Pro Glu Val Gln Phe Asn Trp Tyr Val | |
| | Asp Gly Val Glu Val His Asn Ala Lys Thr Lys | |
| | Pro Arg Glu Glu GIn Phe Asn Ser Thr Tyr Arg | |
| | Val Val Ser Val Leu Thr Val Leu His Gln Asp | |
| | Trp Leu Asn Gly Lys Glu Tyr Lys Cys Lys Val | |
| | Ser Asn Lys Gly Leu Pro Ser Ser Ile Glu Lys | |
| | Thr Ile Ser Lys Ala Lys Gly Gln Pro Arg Glu | |
| | Pro GIn Val Tyr Thr Leu Pro Pro Ser Arg Asp | |
| | Glu Leu Thr Lys Asn Gln Val Ser Leu Thr Cys | |
| | Leu Val Lys Gly Phe Tyr Pro Ser Asp Ile Ala | |
| | Val Glu Trp Glu Ser Asn Gly Gln Pro Glu Asn | |
| | Asn Tyr Lys Thr Thr Pro Pro Val Leu Asp Ser | |
| | Asp Gly Ser Phe Phe Leu Tyr Ser Lys Leu Thr | |
| | Val Asp Lys Ser Arg Trp Gln Gln Gly Asn Val | |
| | Phe Ser Cys Ser Val Met His Glu Ala Leu His | |
| | Asn His Tyr Thr Gln Lys Ser Leu Ser Leu Ser | |
| | Pro Gly Lys | |
| SEQ ID NO: 31 of | Ala Ser Thr Lys Gly Pro Ser Val Phe Pro Leu Ala | 372 |
| WO2014/121087 | Pro Cys Ser Arg Ser Thr Ser Glu Ser Thr Ala Ala | |
| | Leu Gly Cys Leu Val Lys Asp Tyr Phe Pro Glu | |
| | Pro Val Thr Val Ser Trp Asn Ser Gly Ala Leu Thr | |
| | Ser Gly Val His Thr Phe Pro Ala Val Leu Gln Ser | |
| | Ser Gly Leu Tyr Ser Leu Ser Ser Val Val Thr Val | |
| | Pro Ser Ser Ser Leu Gly Thr Lys Thr Tyr Thr Cys | |
| | Asn Val Asp His Lys Pro Ser Asn Thr Lys Val Asp | |
| | Lys Arg Val Glu Ser Lys Tyr Gly Pro Pro Cys | |
| | Pro Pro Cys Pro Ala Pro Pro Val Ala Gly Pro | |
| | Ser Val Phe Leu Phe Pro Pro Lys Pro Lys Asp | |
| | Thr Leu Met Ile Ser Arg Thr Pro Glu Val Thr | |
| | Cys Val Val Val Asp Val Ser Gln Glu Asp Pro | |
| | Glu Val Gln Phe Asn Trp Tyr Val Asp Gly Val | |
| | Glu Val His Asn Ala Lys Thr Lys Pro Arg Glu | |
| | Glu GIn Phe Asn Ser Thr Tyr Arg Val Val Ser | |
| | Val Leu Thr Val Leu His Gln Asp Trp Leu Asn | |
| | Gly Lys Glu Tyr Lys Cys Lys Val Ser Asn Lys | |
| | Gly Leu Pro Ser Ser Ile Glu Lys Thr Ile Ser Lys | |
| | Ala Lys Gly Gln Pro Arg Glu Pro Gln Val Tyr | |
| | Thr Leu Pro Pro Ser Gln Glu Glu Met Thr Lys | |
| | Asn Gln Val Ser Leu Thr Cys Leu Val Lys Gly | |
| | Phe Tyr Pro Ser Asp Ile Ala Val Glu Trp Glu | |
| | Ser Asn Gly Gln Pro Glu Asn Asn Tyr Lys Thr | |
| | Thr Pro Pro Val Leu Asp Ser Asp Gly Ser Phe | |
| | Phe Leu Tyr Ser Arg Leu Thr Val Asp Lys Ser | |
| | Arg Trp Gln Glu Gly Asn Val Phe Ser Cys Ser | |
| | Val Met His Glu Ala Leu His Asn His Tyr Thr | |
| | Gln Lys Ser Leu Ser Leu Ser Leu Gly Lys | |
| SEQ ID NO: 37 of | Ala Ser Thr Lys Gly Pro Ser Val Phe Pro Leu Ala | 373 |
| WO2014/121087 | Pro Ser Ser Lys Ser Thr Ser Gly Gly Thr Ala Ala | |
| | Leu Gly Cys Leu Val Lys Asp Tyr Phe Pro Glu | |
| | Pro Val Thr Val Ser Trp Asn Ser Gly Ala Leu Thr | |
| | Ser Gly Val His Thr Phe Pro Ala Val Leu Gln Ser | |
| | Ser Gly Leu Tyr Ser Leu Ser Ser Val Val Thr Val | |
| | Pro Ser Ser Ser Leu Gly Thr Gln Thr Tyr Ile Cys | |
| | Asn Val Asn His Lys Pro Ser Asn Thr Lys Val Asp | |
| | Lys Lys Val Glu Pro Lys Ser Cys Asp Lys Thr | |
| | His Thr Cys Pro Pro Cys Pro Ala Pro Pro Val | |
| | Ala Gly Pro Ser Val Phe Leu Phe Pro Pro Lys | |
| | Pro Lys Asp Thr Leu Met Ile Ser Arg Thr Pro | |
| | Glu Val Thr Cys Val Val Val Asp Val Ser Gln | |
| | Glu Asp Pro Glu Val Gln Phe Asn Trp Tyr Val | |
| | Asp Gly Val Glu Val His Asn Ala Lys Thr Lys | |
| | Pro Arg Glu Glu GIn Phe Asn Ser Thr Tyr Arg | |
| | Val Val Ser Val Leu Thr Val Leu His Gln Asp | |
| | Trp Leu Asn Gly Lys Glu Tyr Lys Cys Lys Val | |
| | Ser Asn Lys Gly Leu Pro Ser Ser Ile Glu Lys | |
| | Thr Ile Ser Lys Ala Lys Gly Gln Pro Arg Glu | |
| | Pro GIn Val Tyr Thr Leu Pro Pro Ser Arg Asp | |
| | Glu Leu Thr Lys Asn Gln Val Ser Leu Thr Cys | |
| | Leu Val Lys Gly Phe Tyr Pro Ser Asp Ile Ala | |
| | Val Glu Trp Glu Ser Asn Gly Gln Pro Glu Asn | |
| | Asn Tyr Lys Thr Thr Pro Pro Val Leu Asp Ser | |
| | Asp Gly Ser Phe Phe Leu Tyr Ser Lys Leu Thr | |
| | Val Asp Lys Ser Arg Trp Gln Gln Gly Asn Val | |
| | Phe Ser Cys Ser Val Met His Glu Ala Leu His | |
| | Asn Arg Phe Thr Gln Lys Ser Leu Ser Leu Ser | |
| | Pro Gly Lys | |
| SEQ ID NO: 38 of | Ala Ser Thr Lys Gly Pro Ser Val Phe Pro Leu Ala | 374 |
| WO2014/121087 | Pro Cys Ser Arg Ser Thr Ser Glu Ser Thr Ala Ala | |
| | Leu Gly Cys Leu Val Lys Asp Tyr Phe Pro Glu | |
| | Pro Val Thr Val Ser Trp Asn Ser Gly Ala Leu Thr | |
| | Ser Gly Val His Thr Phe Pro Ala Val Leu Gln Ser | |
| | Ser Gly Leu Tyr Ser Leu Ser Ser Val Val Thr Val | |
| | Pro Ser Ser Ser Leu Gly Thr Lys Thr Tyr Thr Cys | |
| | Asn Val Asp His Lys Pro Ser Asn Thr Lys Val Asp | |
| | Lys Arg Val Glu Ser Lys Tyr Gly Pro Pro Cys | |
| | Pro Pro Cys Pro Ala Pro Pro Val Ala Gly Pro | |
| | Ser Val Phe Leu Phe Pro Pro Lys Pro Lys Asp | |
| | Thr Leu Met Ile Ser Arg Thr Pro Glu Val Thr | |
| | Cys Val Val Val Asp Val Ser Gln Glu Asp Pro | |
| | Glu Val GIn Phe Asn Trp Tyr Val Asp Gly Val | |
| | Glu Val His Asn Ala Lys Thr Lys Pro Arg Glu | |
| | Glu GIn Phe Asn Ser Thr Tyr Arg Val Val Ser | |
| | Val Leu Thr Val Leu His Gln Asp Trp Leu Asn | |
| | Gly Lys Glu Tyr Lys Cys Lys Val Ser Asn Lys | |
| | Gly Leu Pro Ser Ser Ile Glu Lys Thr Ile Ser Lys | |
| | Ala Lys Gly Gln Pro Arg Glu Pro Gln Val Tyr | |
| | Thr Leu Pro Pro Ser Gln Glu Glu Met Thr Lys | |
| | Asn Gln Val Ser Leu Thr Cys Leu Val Lys Gly | |
| | Phe Tyr Pro Ser Asp Ile Ala Val Glu Trp Glu | |
| | Ser Asn Gly Gln Pro Glu Asn Asn Tyr Lys Thr | |
| | Thr Pro Pro Val Leu Asp Ser Asp Gly Ser Phe | |
| | Phe Leu Tyr Ser Arg Leu Thr Val Asp Lys Ser | |
| | Arg Trp Gln Glu Gly Asn Val Phe Ser Cys Ser | |
| | Val Met His Glu Ala Leu His Asn Arg Phe Thr | |
| | Gln Lys Ser Leu Ser Leu Ser Leu Gly Lys | |

In a particular embodiment, the IgG4 with reduced effector function comprises the bolded portion of the amino acid sequence of SEQ ID NO:31 of WO2014/121087, sometimes referred to herein as IgG4s or hIgG4s, having the amino acid sequence:

(SEQ ID NO: 396)
ESKYGPPCPPCPAPPVAGPSVFLFPPKPKDTLMISRTPEVTCVVVDVSQE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.8.2. Fc Heterodimerization Variants

Certain IFN proproteins entail dimerization between two Fc domains that, unlike a native immunoglobulin, are operably linked to non-identical N-terminal or C-terminal regions. Inadequate heterodimerization of two Fc domains to form an Fc region has can be an obstacle for increasing the yield of desired heterodimeric molecules and represents challenges for purification. A variety of approaches available in the art can be used in for enhancing dimerization of Fc domains that might be present in the IFN proproteins of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides IFN proproteins comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired IFN proprotein, while homodimerization of identical heavy chains will reduce yield of the desired IFN proprotein. Thus, in a preferred embodiment, the polypeptides that associate to form an IFN proprotein of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285 (25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IFN proproteins comprise a first CH3 domain and a second Ig CH3 domain, wherein the first and second Ig CH3 domains differ from one another by at least one amino acid, and wherein at least one amino acid difference reduces binding of the IFN proprotein to Protein A as compared to a corresponding IFN proprotein lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.8.3. Hinge Domains

The IFN proproteins of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric IFN proprotein formed by the association of two Fc domains) can comprise two associated hinge sequences on separate polypeptide chains. Sometimes, the two associated hinge sequences are referred to as a “hinge region”. In certain embodiments of IFN proproteins, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an IFN proprotein of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the IFN proproteins of the disclosure comprise a modified hinge region that reduces binding affinity for an Fcγ receptor relative to a wild-type hinge region of the same isotype (e.g., human IgG1 or human IgG4).

In one embodiment, the IFN proproteins of the disclosure comprise an Fc region in which each Fc domain possesses an intact hinge domain at its N-terminus, where each Fc domain and hinge domain is derived from IgG4, and each hinge domain comprises the modified sequence CPPC (SEQ ID NO: 375). The core hinge region of human IgG4 contains the sequence CPSC (SEQ ID NO: 376) compared to IgG1 that contains the sequence CPPC (SEQ ID NO: 375). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30 (1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

The hinge sequences incorporated into the IFN proproteins of the disclosure may be full length (“long”) or truncated (“short”). An example of a full-length hinge sequence is ESKYGPPCPPCPAPPVA (SEQ ID NO: 377). An example of a truncated hinge sequence is ESKYGPPCPPC (SEQ ID NO: 378). ESKYGPPCPPC (SEQ ID NO: 378) is truncated by 6 amino acids as compared to the full-length hinge sequence ESKYGPPCPPCPAPPCA (SEQ ID NO: 379). In various aspects, the truncated hinge can have C-terminal deletions of 1, 2, 3, 4, 5 or 6 amino acids as compared to a full-length hinge sequence, e.g., any of the full-length hinge sequences disclosed herein. Without being bound by theory, it is believed that a truncated hinge sequence may confer improved steric constraints on the IFN moiety. In IFN proproteins comprising two hinge domains in each half antibody (e.g., the IFN proproteins having the configurations depicted in FIGS. 1C, 2C and 2F), the two hinge domains can both be full-length, truncated, or a combination thereof. Thus, the N-terminal hinge domains can be truncated, the C-terminal hinge domains can be truncated, or both the N-terminal and C-terminal hinge domains can be truncated.

### 6.8.3.1. Chimeric Hinge Sequences

The hinge domain can be a chimeric hinge domain.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 380) (previously disclosed as SEQ ID NO:8 of WO2014/121087, which is incorporated by reference in its entirety herein) or ESKYGPPCPPCPAPPVA (SEQ ID NO: 377) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.8.1).

### 6.8.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G - - - or - - - with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G - - - (233-236) and no G (233-236). Optionally, the hinge domain amino acid sequence comprises CPPCPAPGGG-GPSVF (SEQ ID NO: 381) (previously disclosed as SEQ ID NO: 1 of WO2016161010A2), CPPCPAPGG--GPSVF (SEQ ID NO: 382) (previously disclosed as SEQ ID NO:2 of WO2016161010A2), CPPCPAPG - - - GPSVF (SEQ ID NO: 383) (previously disclosed as SEQ ID NO:3 of WO2016161010A2), or CPPCPAP - - - GPSVF (SEQ ID NO: 384) (previously disclosed as SEQ ID NO:4 of WO2016161010A2).

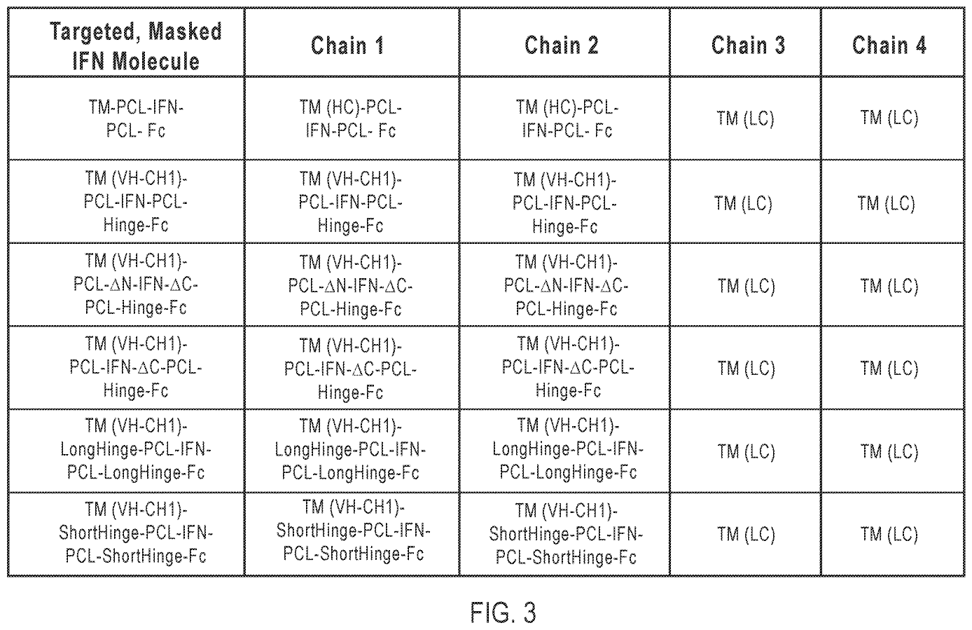

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.8.1).

### 6.9. Nucleic Acids and Host Cells

In another aspect, the disclosure provides nucleic acids encoding the IFN proproteins of the disclosure. In some embodiments, the IFN proproteins are encoded by a single nucleic acid. In other embodiments, the IFN proproteins can be encoded by a plurality (e.g., two, three, four or more) nucleic acids.

A single nucleic acid can encode an IFN proprotein that comprises a single polypeptide chain, an IFN proprotein that comprises two or more polypeptide chains, or a portion of an IFN proprotein that comprises more than two polypeptide chains (for example, a single nucleic acid can encode two polypeptide chains of an IFN proprotein comprising three, four or more polypeptide chains, or three polypeptide chains of an IFN proprotein comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, an IFN proprotein comprising two or more polypeptide chains is encoded by two or more nucleic acids. The number of nucleic acids encoding an IFN proprotein can be equal to or less than the number of polypeptide chains in the IFN proprotein (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.9.1. Vectors

The disclosure provides vectors comprising nucleotide sequences encoding an IFN proprotein or a component thereof described herein, for example one or two of the polypeptide chains of a half antibody of an IFN proprotein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.9.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, Hela cells, COS cells, CHO cells, HEK293 cells, BHK cells and MDCKII cells. Suitable insect cells include, but are not limited to, Sf9 cells.

### 6.10. Pharmaceutical Compositions

The IFN proproteins of the disclosure may be in the form of compositions comprising the IFN proprotein and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the IFN proprotein and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular IFN proprotein, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an IFN proprotein of the disclosure per dose. The quantity of IFN proprotein included in a unit dose will depend on the disease being treated, as well as other factors as are well known in the art. Such unit dosages may be in the form of a lyophilized dry powder containing an amount of IFN proprotein suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of IFN proprotein suitable for a single administration.

The pharmaceutical compositions may also be supplied in bulk from containing quantities of IFN proprotein suitable for multiple administrations.

Pharmaceutical compositions may be prepared for storage as lyophilized formulations or aqueous solutions by mixing an IFN proprotein having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of IFN proprotein.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The IFN proproteins of the disclosure can be formulated as pharmaceutical compositions comprising the IFN proproteins, for example containing one or more pharmaceutically acceptable excipients or carriers. To prepare pharmaceutical or sterile compositions comprising the IFN proproteins of the present disclosure, a IFN proprotein preparation can be combined with one or more pharmaceutically acceptable excipient or carrier.

For example, formulations of IFN proproteins can be prepared by mixing IFN proproteins with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A composition of the present disclosure may also be administered via one or more routes of administration using one or more of a variety of methods known in the art. As will be appreciated by the skilled artisan, the route and/or mode of administration will vary depending upon the desired results. Selected routes of administration for IFN proproteins include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, the IFN proproteins are administered by infusion. In another embodiment, the IFN proprotein of the disclosure is administered subcutaneously.

### 6.10.1. Pharmaceutical Compositions for Delivery of IFN Proprotein Encoding Nucleic Acids

An IFN proprotein (e.g., an IFN receptor agonist) of the disclosure can be delivered by any method useful for gene therapy, for example as mRNA or through viral vectors encoding the IFN proprotein (e.g., IFN receptor agonist) under the control of a suitable promoter.

Exemplary viral vectors include recombinant adenovirus and adeno-associated virus vectors (rAAV). rAAV vectors are based on the defective and nonpathogenic parvovirus adeno-associated type 2 virus. Most such vectors are derived from a plasmid that retains only the AAV inverted terminal repeats flanking the transgene expression cassette. Efficient gene transfer and stable transgene delivery due to integration into the genomes of the transduced cell are key features for this vector system. AAV serotypes useful for delivering IL27 transgenes AAV1, AAV2, AAV3, AAV4, AAV5, AAV6, AAV8, AAV 8.2, AAV9, and AAV rh10 and pseudotyped AAV such as AAV2/8, AAV2/5 and AAV2/6.

AAV may be manufactured at a clinical scale by a number of different processes. Examples of systems that can be used include (1) plasmid DNA transfection in mammalian cells, (2) Ad infection of stable mammalian cell lines, (3) infection of mammalian cells with recombinant herpes simplex viruses (rHSVs), and (4) infection of insect cells (Sf9 cells) with recombinant baculoviruses (reviewed by Penaud-Budloo et al., 2018, Mol Ther Methods Clin Dev. 8:166-180).

Replication-deficient recombinant adenoviral vectors (Ad) can be produced at high titer and readily infect a number of different cell types. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Packaging cells are used to form virus particles that are capable of infecting a host cell. Such cells include 293 cells, which package adenovirus, and w2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

The nucleic acid molecule (e.g., mRNA) or virus can be formulated as the sole pharmaceutically active ingredient in a pharmaceutical composition or can be combined with other active agents for the particular disorder treated. Optionally, other medicinal agents, pharmaceutical agents, carriers, adjuvants, diluents can be included in the compositions provided herein. For example, any one or more of a wetting agents, emulsifiers and lubricants, such as sodium lauryl sulfate and magnesium stearate, as well as coloring agents, release agents, coating agents, sweetening, flavoring and perfuming agents, preservatives, antioxidants, chelating agents and inert gases also can be present in the compositions. Exemplary other agents and excipients that can be included in the compositions include, for example, water soluble antioxidants, such as ascorbic acid, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid and phosphoric acid.

### 6.11. Therapeutic Indications and Methods of Use

The present disclosure provides methods for using and applications for the IFN proproteins of the disclosure.

The IFN proproteins (e.g. IFN receptor agonists) of the disclosure can be used to stimulate the immune response in a variety of applications.

In certain aspects, the disclosure provides a method of treating cancer, comprising administering to a subject in need thereof an IFN proprotein or pharmaceutical composition as described herein. In some embodiments, an activated IFN protein comprising the IFN moiety is produced by cleavage of one or more protease-cleavable linkers in the IFN proprotein by one or more proteases expressed by the cancer tissue. Accordingly, the IFN proprotein is selectively activated in the cancer tissue.

In some embodiments, the disclosure provides a method of treating cancer with an IFN protein that is selectively activated in cancer tissue, comprising administering to a subject in need thereof an IFN proprotein or pharmaceutical composition as described herein, where the IFN proprotein has one or more protease-cleavable linkers, each comprising one or more substrates for one or more proteases expressed by cancer tissue to which the IFN protein is intended. Thus, an activated IFN protein comprising the IFN moiety is produced by cleavage of one or more protease-cleavable linkers in the IFN proprotein by one or more proteases in the cancer tissue.

The present disclosure further provides a method of localized delivery of an IFN protein, comprising administering to a subject an IFN proprotein or pharmaceutical composition as described herein, where the IFN proprotein has one or more protease-cleavable linkers, each comprising one or more substrates for one or more proteases expressed by a tissue to which the IFN protein is to be locally delivered. As used herein, the term “locally delivered” does not require local administration but rather indicates that the active component of the IFN proprotein refers to activation of the protein at a locale of interest by a protease active at the intended site, optionally in conjunction with targeting to the locale of interest with a targeting moiety that recognize a target molecule expressed by the tissue.

The present disclosure further provides a method of administering to the subject IFN therapy with reduced systemic exposure and/or reduced systemic toxicity, comprising administering to a subject the IFN therapy in the form of an IFN proprotein or pharmaceutical composition as described herein, where the IFN proprotein has one or more protease-cleavable linkers, each comprising one or more substrates for one or more proteases expressed by a tissue for which IFN therapy is desirable and/or intended.

Accordingly, the foregoing methods permit IFN therapy with reduced off-target side effects by virtue of preferential activation of an IFN proprotein at a locale intended for IFN treatment.

In some embodiments of the foregoing methods, the IFN proprotein is also targeted and comprises one or more targeting moieties that recognize a target molecule expressed in the locale (e.g., by the tissue) intended for treatment.

Accordingly, the present disclosure provides a method of targeted delivery of an activated IFN protein to a locale intended for treatment, e.g., cancer tissue, comprising administering to a subject an IFN proprotein or pharmaceutical composition as described herein, wherein the IFN comprises one or more targeting moieties that recognize a target molecule expressed in the locale or by the tissue intended for treatment (e.g., cancer tissue) and which has one or more protease-cleavable linkers, each comprising one or more substrates for one or more proteases expressed by a tissue for which IFN therapy is desirable and/or intended.

The present disclosure further provides method of locally inducing an immune response in a target tissue, comprising administering to a subject IFN proprotein or pharmaceutical composition as described herein which has one or more targeting moieties capable of binding a target molecule expressed in the target tissue and one or more protease-cleavable linkers, each comprising one or more substrates for one or more proteases expressed in the target tissue. An activated IFN protein comprising the IFN moiety can then be produced by cleavage of one or more protease-cleavable linkers in the IFN proprotein by one or more proteases in the target tissue. The resulting activated IFN protein can then induce the immune response against at least one cell type in the target tissue.

In some embodiments, the administration is not local to the tissue. For example, when the target tissue is cancer tissue, the administration can be systemic or subcutaneous.

The IFN proproteins of the disclosure can be used in the treatment of any proliferative disorder (e.g., cancer) that expresses a target molecule (either on the tumor cells or in the tumor microenvironment, e.g., the extracellular matrix or the tumor lymphocytes). In particular embodiments, the cancer is acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor.

Table I below shows exemplary indications for which IFN proproteins targeting particular target molecules can be used.

TABLE I
Examples of Target Molecule Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate | ovarian, breast, renal, lung, colorectal, brain cancers |
| receptor alpha | |
| Folate | ovarian cancer |
| receptor beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Additional target molecules and corresponding indications are disclosed in, e.g., Hafeez et al., 2020, Molecules 25:4764, doi: 10.3390/molecules25204764, particularly in Table 1. Table 1 is incorporated by reference in its entirety here.

In further embodiments, the IFN proproteins (e.g., IFN receptor agonists) can be used to enhance an immune response elicited by another agent. Thus, in some embodiments an IFN proprotein (e.g., an IFN receptor agonist) of the disclosure is administered as an adjunct therapy with an immunogenic agent. In some embodiments, the immunogenic agent is an adjuvanted or unadjuvanted vaccine. The IFN proproteins (e.g., IFN receptor agonists) can thus enhance an antigen-specific immune response elicited by the vaccine. In various embodiments, the vaccine is a prophylactic or therapeutic cancer vaccine or a prophylactic or therapeutic vaccine against an infectious agent, e.g., a virus, bacteria, or parasite.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below.

In the numbered embodiments that follow, the targeting moiety preferably binds to a mammalian target molecule, the IFN moiety is preferably derived from a mammalian IFN, the Fc domains are preferably derived from a mammalian antibody, and the subjects are preferably mammals. More preferably, the mammal is human.

1. A Type I interferon (IFN) proprotein, comprising
(a) a first polypeptide chain comprising:
   (i) a first immunoglobulin constant domain;
   (ii) a first linker;
   (iii) a first Type I interferon (IFN) moiety;
   (iv) a second linker; and
   (v) a first Fc domain;
(b) a second polypeptide chain comprising:
   (i) a second immunoglobulin constant domain;
   (ii) a third linker;
   (iii) a second Type I interferon (IFN) moiety;
   (iv) a fourth linker; and
   (v) a second Fc domain associated with the first Fc domain to form an Fc region;
wherein at least two of the first linker, second linker, third linker and fourth linker are protease-cleavable linkers (PCLs), optionally wherein the IFN moieties in the IFN proprotein are sterically hindered from binding to an IFN receptor by the Fc domains.

2. The IFN proprotein of embodiment 1, wherein the first and second IFN moieties each comprise an amino acid sequence having at least about 90% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.

3. The IFN proprotein of embodiment 1, wherein the first and second IFN moieties each comprise an amino acid sequence having about 95% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.

4. The IFN proprotein of embodiment 1, wherein the first and second IFN moieties each comprise an amino acid sequence having about 98% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.

5. The IFN proprotein of any one of embodiments 1 to 4, wherein the first and second IFN moieties each comprise an amino acid sequence having one or more attenuating mutations as compared to mature human IFNα1 or IFNα2b.

6. The IFN proprotein of any one of embodiments 1 to 5, which has one or more mutations selected from L26A, F27A, R33A, R33K, L30A, D35E, H57Y, E58N, Q61S, H57S, E58S, H57A, E58A, Q61A, Q90A, E96A, R120A, L135A, R144A, R144S, R144T, R144Y, R1441, R144L, A145D, A145H, A145K, A145M, A145V, A145Y, R149A, R149K, S152A, R162A, and E165D.

7 The IFN proprotein of any one of embodiments 1 to 6, which comprises the amino acid substitution R33A.

8. The IFN proprotein of any one of embodiments 1 to 6, which comprises the amino acid substitution R33K.

9. The IFN proprotein of any one of embodiments 1 to 6, which comprises the amino acid substitution Q90A.

10. The IFN proprotein of any one of embodiments 1 to 6, which comprises the amino acid substitution E96A.

11. The IFN proprotein of any one of embodiments 1 to 6, which comprises the amino acid substitution R120A.

12. The IFN proprotein of any one of embodiments 1 to 6, which comprises the amino acid substitution A145M.

13. The IFN proprotein of any one of embodiments 1 to 6, which comprises the amino acid substitution R149A.

14. The IFN proprotein of any one of embodiments 1 to 6, which comprises the amino acid substitution R149K.

15. The IFN proprotein of any one of embodiments 1 to 6, which comprises the amino acid substitution S152A.

16. The IFN proprotein of any one of embodiments 1 to 6, which comprises the amino acid substitutions R33A, H57Y, E58N and Q61S.

17. The IFN proprotein of any one of embodiments 1 to 6, which comprises the amino acid substitutions H57Y, E58N, Q61S and R144A.

18. The IFN proprotein of any one of embodiments 1 to 6, which comprises the amino acid substitutions Q90A and R120A.

19. The IFN proprotein of any one of embodiments 1 to 6, which comprises the amino acid substitutions A145M and R149K.

20. The IFN proprotein of any one of embodiments 1 to 19, wherein the first linker, second linker, third linker and fourth linker are protease-cleavable linkers (PCLs).

21. The IFN proprotein of any one of embodiments 1 to 19, wherein the first linker and third linker are protease-cleavable linkers (PCLs), optionally wherein the second linker and fourth linker are non-cleavable linkers (NCLs).

22. The IFN proprotein of any one of embodiments 1 to 19, wherein the second linker and fourth linker are protease-cleavable linkers (PCLs) optionally wherein the first linker and third linker are non-cleavable linkers (NCLs).

23. The IFN proprotein of any one of embodiments 1 to 22, wherein the PCLs each comprise a substrate sequence cleavable by any protease set forth in Table A.

24. The IFN proprotein of any one of embodiments 1 to 23, wherein the PCLs each comprise one or more substrate sequences selected from the substate sequences set forth in Table B.

25. The IFN proprotein of any one of embodiments 1 to 24, wherein the PCLs each comprise one or more spacer sequences selected from the substate sequences set forth in Table C.

26. The IFN proprotein of any one of embodiments 1 to 25, wherein the PCLs each comprise the amino acid sequence of any of the PCL sequences set forth in Table D or a variant thereof with up to 5 amino acid substitutions, e.g., a variant thereof with 1 amino acid substitution, 2 amino acid substitutions, 3 amino acid substitutions, 4 amino acid substitutions, or 5 amino acid substitutions.

27. The IFN proprotein of any one of embodiments 1 to 26, which comprises two or four PCLs comprising or consisting of the amino acid sequence ISSGLLSGRSDNH.

28. The IFN proprotein of any one of embodiments 1 to 26, which comprises two or four PCLs comprising or consisting of the amino acid sequence

GGGISSGLLSGRSDNHGGGISSGLLSGRSDNHGGS.

29. The IFN proprotein of any one of embodiments 1 to 26, which comprises two or four PCLs comprising or consisting of the amino acid sequence

GGSGGSIPVSLRSGGGISSGLLSGRSDNHGGSGGS.

30. The IFN proprotein of any one of embodiments 1 to 26, which comprises two or four PCLs comprising or consisting of the amino acid sequence

GGSGGSVPLSLYSGGGISSGLLSGRSDNHGGSGGS.

31. The IFN proprotein of any one of embodiments 1 to 26, which comprises two or four PCLs comprising or consisting of the amino acid sequence

GGSHPVGLLARGGGHPVGLLARGGGHPVGLLARGS.

32. The IFN proprotein of any one of embodiments 1 to 26, which comprises two or four PCLs comprising or consisting of the amino acid sequence

GGSHPVGLLARGGGHPVGLLARGGSGRSAGGSGRSA.

33. The IFN proprotein of any one of embodiments 1 to 32, wherein the first and third linkers are identical and/or the third and fourth linkers are identical.

34. The IFN proprotein of embodiment 33, wherein the first, second, third and fourth linkers are identical.

35. The IFN proprotein of any one of embodiments 1 to 33, wherein (i) the first and third linkers are non-cleavable linkers or (ii) the second and fourth linkers are non-cleavable linkers.

36. The IFN proprotein of embodiment 35, wherein the non-cleavable linkers comprise or consist of any of the NCL sequences set forth in Table E.

37. The IFN proprotein of any one of embodiments 1 to 33, wherein the first Fc domain and/or the second Fc domain comprises a hinge domain.

38. The IFN proprotein of any one of embodiments 1 to 37, which further comprises one or more targeting moieties that bind to one or more target molecules.

39. The IFN proprotein of embodiment 38, which comprises a first targeting moiety and a second targeting moiety.

40. The IFN proprotein of embodiment 39, wherein the first targeting moiety and second targeting moiety are antibodies or antigen-binding fragments thereof.

41. The IFN proprotein of embodiment 40, wherein the first targeting moiety and second targeting moiety are Fabs.

42. The IFN proprotein of embodiment 40 or embodiment 41, wherein the first targeting moiety and second targeting moiety comprise Fab domains.

43. The IFN proprotein of embodiment 42, wherein the first targeting moiety and second targeting moiety further comprise hinge sequences.

44. The IFN proprotein of embodiment 43, wherein the first targeting moiety and second targeting moiety further comprise Fc domains, each comprising a CH2 domain and a CH3 domain.

45. The IFN proprotein of embodiment 44, wherein the Fc domain of the first targeting moiety and the Fc domain of the second targeting moiety are associated with one another.

46. The IFN proprotein of any one of embodiments 39 to 45, wherein the first and second targeting moieties are N-terminal to the first and third linkers, respectively.

47. The IFN proprotein of any one of embodiments 38 to 46, wherein the first immunoglobulin constant domain is part of the first targeting moiety and the second immunoglobulin constant domain is part of the second targeting moiety.

48. The IFN proprotein of embodiment 40, wherein the first immunoglobulin constant domain is a CH3 domain.

49. The IFN proprotein of embodiment 40, wherein the first immunoglobulin constant domain is a CH1 domain.

50. The IFN proprotein of any one of embodiments 1 to 49, which is configured as illustrated in FIG. 1A, FIG. 2A or FIG. 2D.

51. The IFN proprotein of embodiment 50, which comprises the first polypeptide chain, the second polypeptide chain, a third polypeptide chain and a fourth polypeptide chain, wherein:
(a) the first polypeptide chain comprises:
   (i) a first VH1 domain;
   (ii) a first CH1 domain;
   (iii) a third Fc domain comprising a hinge domain, a CH2 domain and a CH3 domain, wherein the CH3 domain is the first immunoglobulin constant domain;
   (iv) the first linker;
   (v) the first Type I interferon (IFN) moiety;
   (vi) the second linker; and
   (vii) the first Fc domain;
(b) the second polypeptide chain comprises;
   (i) a second VH1 domain;
   (ii) a second CH1 domain;
   (iii) a fourth Fc domain comprising a hinge domain, a CH2 domain and a CH3 domain, wherein the CH3 domain is the second immunoglobulin constant domain;
   (iv) the third linker;
   (v) the second Type I interferon (IFN) moiety;
   (vi) the fourth linker; and
   (vii) the third Fc domain;
(c) the third polypeptide chain comprises:
   (i) a first VL domain;
   (ii) a first CL domain; and
(d) the fourth polypeptide chain comprises:
   (i) a second VL domain; and
   (ii) a second CL domain;
wherein the first polypeptide chain is associated with the third polypeptide chain such that the first VH, CH1, VL and CL form the first targeting moiety and wherein the second polypeptide chain is associated with the fourth polypeptide chain such that the second VH, CH1, VL and CL form the second targeting moiety.

52. The IFN proprotein of embodiment 51, wherein the first, second, third and fourth linkers are protease-cleavable linkers (PCLs).

53. The IFN proprotein of embodiment 51, wherein the first and third linkers are non-cleavable linkers (NCLs) and the second and fourth linkers are protease-cleavable linkers (PCLs).

54. The IFN proprotein of embodiment 51, wherein the first and third linkers are protease-cleavable linkers (PCLs) and the second and fourth linkers are non-cleavable linkers (NCLs).

55. The IFN proprotein of any one of embodiments 1 to 49, which is configured as illustrated in FIG. 1B, FIG. 2B or FIG. 2E.

56. The IFN proprotein of embodiment 55, wherein:
(a) the first polypeptide chain comprises:
   (i) a first VH1 domain;
   (ii) a first CH1 domain;
   (iii) the first linker;
   (iv) the first Type I interferon (IFN) moiety;
   (v) the second linker; and
   (vi) the first Fc domain;
(b) the second polypeptide chain comprises;
   (i) a second VH1 domain;
   (ii) a second CH1 domain;
   (iii) the third linker;
   (iv) the second Type I interferon (IFN) moiety;
   (v) the fourth linker; and
   (vi) the second Fc domain;
(c) the third polypeptide chain comprises:
   (i) a first VL domain;
   (ii) a second CL domain; and
(d) the fourth polypeptide chain comprises:
   (i) a second VL domain;
   (ii) a second CL domain;
wherein the first polypeptide chain is associated with the third polypeptide chain such that the first VH, CH1, VL and CL form the first targeting moiety and wherein the second polypeptide chain is associated with the fourth polypeptide chain such that the second VH, CH1, VL and CL form the second targeting moiety.

57. The IFN proprotein of embodiment 56, wherein the first, second, third and fourth linkers are protease-cleavable linkers (PCLs).

58. The IFN proprotein of embodiment 56, wherein the first and third linkers are non-cleavable linkers (NCLs) and the second and fourth linkers are protease-cleavable linkers (PCLs).

59. The IFN proprotein of embodiment 56, wherein the first and third linkers are protease-cleavable linkers (PCLs) and the second and fourth linkers are non-cleavable linkers (NCLs).

60. The IFN proprotein of any one of embodiments 1 to 49, which is configured as illustrated in FIG. 1C, FIG. 2C or FIG. 2F.

61. The IFN proprotein of embodiment 60, which comprises the first polypeptide chain, the second polypeptide chain, a third polypeptide chain and a fourth polypeptide chain, wherein:
(a) the first polypeptide chain comprises:
   (i) a first VH1 domain;
   (ii) a first CH1 domain;
   (iii) a first hinge domain;
   (iv) the first linker;
   (v) the first Type I interferon (IFN) moiety;
   (vi) the second linker; and
   (vii) the first Fc domain;
(b) the second polypeptide chain comprises;
   (i) a second VH1 domain;
   (ii) a second CH1 domain;
   (iii) a second hinge domain;
   (iv) the third linker;
   (v) the second Type I interferon (IFN) moiety;
   (vi) the fourth linker; and
   (vii) the second Fc domain;
(c) the third polypeptide chain comprises:
   (i) a first VL domain;
   (ii) a first CL domain; and
(d) the fourth polypeptide chain comprises:
   (i) a second VL domain; and
   (ii) a second CL domain;
wherein the first polypeptide chain is associated with the third polypeptide chain such that the first VH, CH1, VL and CL form the first targeting moiety and wherein the second polypeptide chain is associated with the fourth polypeptide chain such that the second VH, CH1, VL and CL form the second targeting moiety.

62. The IFN proprotein of embodiment 61, wherein the first, second, third and fourth linkers are protease-cleavable linkers (PCLs).

63. The IFN proprotein of embodiment 61, wherein the first and third linkers are non-cleavable linkers (NCLs) and the second and fourth linkers are protease-cleavable linkers (PCLs).

64. The IFN proprotein of embodiment 61, wherein the first and third linkers are protease-cleavable linkers (PCLs) and the second and fourth linkers are non-cleavable linkers (NCLs).

65. The IFN proprotein of any one of embodiments 38 to 61, herein the first targeting moiety and/or second targeting moiety is capable of binding to an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

66. The IFN proprotein of any one of embodiments 38 to 65, wherein the first targeting moiety and/or second targeting moiety is capable of binding to any target molecule identified in Section 6.7.

67. The IFN proprotein of any one of embodiments 38 to 66, wherein the first targeting moiety and/or second targeting moiety (a) comprises the (i) CDR or (ii) VH and VL sequences of antibody set forth in Table F or (b) competes with the antibody set forth in Table F for binding to the target molecule.

68. The IFN proprotein of any one of embodiments 38 to 66, wherein the first targeting moiety and/or second targeting moiety is capable of binding to an ECM antigen which is optionally selected from syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

69. The IFN proprotein of embodiment 68, wherein the first targeting moiety and/or second targeting moiety is capable of binding to a nectin, e.g., nectin 4.

70. The IFN proprotein of embodiment 68, wherein the first targeting moiety and/or second targeting moiety is capable of binding to a collagen, e.g., collagen X.

71. The IFN proprotein of any one of embodiments 38 to 66, wherein the first targeting moiety and/or second targeting moiety is capable of binding to a cell surface molecule of tumor or viral lymphocytes.

72. The IFN proprotein of embodiment 71, wherein the antigen is a T-cell co-stimulatory protein.

73. The IFN proprotein of embodiment 72, wherein the T-cell co-stimulatory protein is CD27, CD28, 4-1BB (CD137), OX40, CD30, CD40, ICOS, lymphocyte function-associated antigen-1 (LFA-1), CD2, CD7, LIGHT, NKG2C, or B7-H3.

74. The IFN proprotein of embodiment 73, wherein the T-cell co-stimulatory protein is B7-H3.

75. The IFN proprotein of any one of embodiments 38 to 66, wherein the first targeting moiety and/or second targeting moiety is capable of binding to a checkpoint inhibitor.

76. The IFN proprotein of embodiment 75, wherein the checkpoint inhibitor is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.

77. The IFN proprotein of embodiment 76, wherein the checkpoint inhibitor is PDL1.

78. The IFN proprotein of embodiment 76, wherein the checkpoint inhibitor is PD1.

79 The IFN proprotein of embodiment 76, wherein the checkpoint inhibitor is LAG3.

80. The IFN proprotein of any one of embodiments 38 to 66, wherein the first targeting moiety and/or second targeting moiety is capable of binding to a tumor-associated antigen (TAA).

81. The IFN proprotein of embodiment 80, wherein the first targeting moiety and/or second targeting moiety is capable of binding to AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvlll, ErbB2/Her2, ErbB3, ErbB4, ETV6-AML, EpCAM, EphA2, Fra-1, FOLR1, a GAGE protein (e.g., GAGE-1 or -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

82. The IFN proprotein of embodiment 81, wherein the TAA is EGFR.

83. The IFN proprotein of embodiment 81, wherein the TAA is HER2.

84. The IFN proprotein of embodiment 81, wherein the TAA is EPCAM.

85. The IFN proprotein of embodiment 81, wherein the TAA is CEACAM5.

86. The IFN proprotein of embodiment 81, wherein the TAA is CD20.

87. The IFN proprotein of any one of embodiments 38 to 66, wherein the first targeting moiety and/or second targeting moiety is capable of binding to a dendritic cell (DC) antigen which is optionally selected from XCR1, Clec9a, CD1c, CD11c, CD14, PDL1, macrophage mannose receptor (CD206), and DEC-205.

88. The IFN proprotein of embodiment 87, wherein the dendritic cell antigen is XCR1.

89. The IFN proprotein of embodiment 87, wherein the dendritic cell antigen is Clec9a.

90. The IFN proprotein of embodiment 87, wherein the dendritic cell antigen is DEC-205.

91. The IFN proprotein of any one of embodiments 38 to 66, wherein the first targeting moiety and/or second targeting moiety is capable of binding to a natural killer (NK) cell antigen.

92. The IFN proprotein of any one of embodiments 1 to 37, which further comprises one or more targeting moieties each comprising means for binding to one or more target molecules.

93. The IFN proprotein of embodiment 92, which comprises a first targeting moiety and a second targeting moiety, each comprising means for binding to a target molecule.

94. The IFN proprotein of embodiment 93, wherein the first targeting moiety and second targeting moiety are antibodies or antigen-binding fragments thereof.

95. The IFN proprotein of embodiment 94, wherein the first targeting moiety and second targeting moiety are Fabs.

96. The IFN proprotein of embodiment 94, wherein the first targeting moiety and second targeting moiety comprise Fab domains.

97. The IFN proprotein of any one of embodiments 92 to 96, wherein the first targeting moiety and second targeting moiety further comprise hinge sequences.

98. The IFN proprotein of embodiment 97, wherein the first targeting moiety and second targeting moiety further comprise Fc domains, each comprising a CH2 domain and a CH3 domain.

99. The IFN proprotein of embodiment 98, wherein the Fc domain of the first targeting moiety and the Fc domain of the second targeting moiety are associated with one another.

100. The IFN proprotein of any one of embodiments 92 to 99, wherein the first and second targeting moieties are N-terminal to the first and third linkers, respectively.

101. The IFN proprotein of any one of embodiments 92 to 100, wherein the first immunoglobulin constant domain is part of the first targeting moiety and the second immunoglobulin constant domain is part of the second targeting moiety.

102. The IFN proprotein of embodiment 101, wherein the first immunoglobulin constant domain is a CH3 domain.

103. The IFN proprotein of embodiment 101, wherein the first immunoglobulin constant domain is a CH1 domain.

104. The IFN proprotein of any one of embodiments 92 to 103, which is configured as illustrated in FIG. 1A, FIG. 2A or FIG. 2D.

105. The IFN proprotein of embodiment 104, which comprises the first polypeptide chain, the second polypeptide chain, a third polypeptide chain and a fourth polypeptide chain, wherein:
(a) the first polypeptide chain comprises:
   (i) a first VH1 domain;
   (ii) a first CH1 domain;
   (iii) a third Fc domain comprising a hinge domain, a CH2 domain and a CH3 domain, wherein the CH3 domain is the first immunoglobulin constant domain;
   (iv) the first linker;
   (v) the first Type I interferon (IFN) moiety;
   (vi) the second linker; and
   (vii) the first Fc domain;
(b) the second polypeptide chain comprises;
   (i) a second VH1 domain;
   (ii) a second CH1 domain;
   (iii) a fourth Fc domain comprising a hinge domain, a CH2 domain and a CH3 domain, wherein the CH3 domain is the second immunoglobulin constant domain;
   (iv) the third linker;
   (v) the second Type I interferon (IFN) moiety;
   (vi) the fourth linker; and
   (vii) the third Fc domain;
(c) the third polypeptide chain comprises:
   (i) a first VL domain;
   (ii) a first CL domain; and
(d) the fourth polypeptide chain comprises:
   (i) a second VL domain; and
   (ii) a second CL domain;
wherein the first polypeptide chain is associated with the third polypeptide chain such that the first VH, CH1, VL and CL form the first targeting moiety and wherein the second polypeptide chain is associated with the fourth polypeptide chain such that the second VH, CH1, VL and CL form the second targeting moiety.

106. The IFN proprotein of embodiment 105, wherein the first, second, third and fourth linkers are protease-cleavable linkers (PCLs).

107. The IFN proprotein of embodiment 105, wherein the first and third linkers are non-cleavable linkers (NCLs) and the second and fourth linkers are protease-cleavable linkers (PCLs).

108. The IFN proprotein of embodiment 105, wherein the first and third linkers are protease-cleavable linkers (PCLs) and the second and fourth linkers are non-cleavable linkers (NCLs).

109. The IFN proprotein of any one of embodiments 92 to 103, which is configured as illustrated in FIG. 1B, FIG. 2B or FIG. 2E.

110. The IFN proprotein of embodiment 109, wherein:
(a) the first polypeptide chain comprises:
   (i) a first VH1 domain;
   (ii) a first CH1 domain;
   (iii) the first linker;
   (iv) the first Type I interferon (IFN) moiety;
   (v) the second linker; and
   (vi) the first Fc domain;
(b) the second polypeptide chain comprises;
   (i) a second VH1 domain;
   (ii) a second CH1 domain;
   (iii) the third linker;
   (iv) the second Type I interferon (IFN) moiety;
   (v) the fourth linker; and
   (vi) the second Fc domain;
(c) the third polypeptide chain comprises:
   (i) a first VL domain;
   (ii) a second CL domain; and
(d) the fourth polypeptide chain comprises:
   (i) a second VL domain;
   (ii) a second CL domain;
wherein the first polypeptide chain is associated with the third polypeptide chain such that the first VH, CH1, VL and CL form the first targeting moiety and wherein the second polypeptide chain is associated with the fourth polypeptide chain such that the second VH, CH1, VL and CL form the second targeting moiety.

111. The IFN proprotein of embodiment 110, wherein the first, second, third and fourth linkers are protease-cleavable linkers (PCLs).

112. The IFN proprotein of embodiment 110, wherein the first and third linkers are non-cleavable linkers (NCLs) and the second and fourth linkers are protease-cleavable linkers (PCLs).

113. The IFN proprotein of embodiment 110, wherein the first and third linkers are protease-cleavable linkers (PCLs) and the second and fourth linkers are non-cleavable linkers (NCLs).

114. The IFN proprotein of any one of embodiments 92 to 103, which is configured as illustrated in FIG. 1C, FIG. 2C or FIG. 2F.

115. The IFN proprotein of embodiment 114, which comprises the first polypeptide chain, the second polypeptide chain, a third polypeptide chain and a fourth polypeptide chain, wherein:
(a) the first polypeptide chain comprises:
   (i) a first VH1 domain;
   (ii) a first CH1 domain;
   (iii) a first hinge domain;
   (iv) the first linker;
   (v) the first Type I interferon (IFN) moiety;
   (vi) the second linker; and
   (vii) the first Fc domain;
(b) the second polypeptide chain comprises;
   (i) a second VH1 domain;
   (ii) a second CH1 domain;
   (iii) a second hinge domain;
   (iv) the third linker;
   (v) the second Type I interferon (IFN) moiety;
   (vi) the fourth linker; and
   (vii) the second Fc domain;
(c) the third polypeptide chain comprises:
   (i) a first VL domain;
   (ii) a first CL domain; and
(d) the fourth polypeptide chain comprises:
   (i) a second VL domain; and
   (ii) a second CL domain;
wherein the first polypeptide chain is associated with the third polypeptide chain such that the first VH, CH1, VL and CL form the first targeting moiety and wherein the second polypeptide chain is associated with the fourth polypeptide chain such that the second VH, CH1, VL and CL form the second targeting moiety.

116. The IFN proprotein of embodiment 115, wherein the first, second, third and fourth linkers are protease-cleavable linkers (PCLs).

117. The IFN proprotein of embodiment 115, wherein the first and third linkers are non-cleavable linkers (NCLs) and the second and fourth linkers are protease-cleavable linkers (PCLs).

118. The IFN proprotein of embodiment 115, wherein the first and third linkers are protease-cleavable linkers (PCLs) and the second and fourth linkers are non-cleavable linkers (NCLs).

119. The IFN proprotein of any one of embodiments 92 to 119, wherein the first targeting moiety and/or the second targeting moiety comprises means for binding to an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

120. The IFN proprotein of any one of embodiments 92 to 119, wherein the first targeting moiety and/or second targeting moiety comprises means for binding a target molecule identified in Section 6.6.

121. The IFN proprotein of any one of embodiments 92 to 119, wherein the first targeting moiety and/or second targeting moiety comprises means for binding to an ECM antigen which is optionally selected from syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

122. The IFN proprotein of embodiment 121, wherein the first targeting moiety and/or second targeting moiety comprises means for binding to a nectin, e.g., nectin 4.

123. The IFN proprotein of embodiment 121, wherein the first targeting moiety and/or second targeting moiety comprises means for binding to a collagen, e.g., collagen X.

124. The IFN proprotein of any one of embodiments 92 to 119, wherein the first targeting moiety and/or second targeting moiety comprises means for binding to a cell surface molecule of tumor or viral lymphocytes.

125. The IFN proprotein of embodiment 124, wherein the cell surface molecule is a T-cell co-stimulatory protein.

126. The IFN proprotein of embodiment 125, wherein the T-cell co-stimulatory protein is CD27, CD28, 4-1BB (CD137), OX40, CD30, CD40, ICOS, lymphocyte function-associated antigen-1 (LFA-1), CD2, CD7, LIGHT, NKG2C, or B7-H3.

127. The IFN proprotein of embodiment 126, wherein the T-cell co-stimulatory protein is B7-H3.

128. The IFN proprotein of any one of embodiments 92 to 119, wherein the first targeting moiety and/or second targeting moiety comprises means for binding to a checkpoint inhibitor.

129. The IFN proprotein of embodiment 128, wherein the checkpoint inhibitor is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.

130. The IFN proprotein of embodiment 129, wherein the checkpoint inhibitor is PDL1.

131. The IFN proprotein of embodiment 129, wherein the checkpoint inhibitor is PD1.

132. The IFN proprotein of embodiment 129, wherein the checkpoint inhibitor is LAG3.

133. The IFN proprotein of any one of embodiments 92 to 119, wherein the first targeting moiety and/or second targeting moiety comprises means for binding to a tumor-associated antigen (TAA).

134. The IFN proprotein of embodiment 133, wherein the TAA is AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, a GAGE protein (e.g., GAGE-1 or -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

135. The IFN proprotein of embodiment 134, wherein the TAA is EGFR.

136. The IFN proprotein of embodiment 134, wherein the TAA is HER2.

137. The IFN proprotein of embodiment 134, wherein the TAA is EPCAM.

138. The IFN proprotein of embodiment 134, wherein the TAA is CEACAM5.

139. The IFN proprotein of embodiment 134, wherein the TAA is CD20.

140. The IFN proprotein of any one of embodiments 92 to 119, wherein the first targeting moiety and/or second targeting moiety comprises means for binding to a dendritic cell (DC) antigen which is optionally selected from XCR1, Clec9a, CD1c, CD11c, CD14, PDL1, macrophage mannose receptor (CD206), and DEC-205.

141. The IFN proprotein of embodiment 140, wherein the dendritic cell antigen is XCR1.

142. The IFN proprotein of embodiment 140, wherein the dendritic cell antigen is Clec9a.

143. The IFN proprotein of embodiment 140, wherein the dendritic cell antigen is DEC-205.

144. The IFN proprotein of any one of embodiments 92 to 119, wherein the first targeting moiety and/or second targeting moiety comprises means for binding to a natural killer (NK) cell antigen.

145. The IFN proprotein of any one of embodiments 1 to 144, wherein the Fc region is homodimeric.

146. The IFN proprotein of any one of embodiments 1 to 144, wherein the Fc region is heterodimeric.

147. A nucleic acid or plurality of nucleic acids encoding the IFN proprotein of any one of embodiments 1 to 146.

148. A host cell engineered to express the IFN proprotein of any one of embodiments 1 to 146 or the nucleic acid(s) of embodiment 147.

149. A method of producing the IFN proprotein of any one of embodiments 1 to 146, comprising culturing the host cell of embodiment 148 and recovering the IFN proprotein expressed thereby.

150. A pharmaceutical composition comprising the IFN proprotein of any one of embodiments 1 to 146 and an excipient.

151. A method of treating cancer, comprising administering to a subject in need thereof the IFN proprotein of any one of embodiments 1 to 146 or the pharmaceutical composition of embodiment 150.

152. The method of embodiment 151, wherein the IFN proprotein comprises at least one targeting moiety that is capable of binding to a target molecule.

153. The method of embodiment 151, wherein the IFN proprotein comprises at least one means for binding a target molecule.

154. The method of any one of embodiments 150 to 153, wherein the cancer is associated with expression of the target molecule, e.g., a TAA and associated cancer as set forth in Table I.

155. The method of any one of embodiments 150 to 154, wherein an activated IFN protein comprising the IFN moiety is produced by cleavage of one or more protease-cleavable linkers in the IFN proprotein by one or more proteases expressed by the cancer tissue.

156. The method of embodiment 155, wherein the IFN protein is selectively activated in the cancer tissue.

157. A method of localized delivery of an IFN protein, comprising administering to a subject an IFN proprotein according to any one of embodiments 1 to 146 (or a pharmaceutical composition comprising the IFN proprotein and an excipient) which has one or more protease-cleavable linkers, each comprising one or more substrates for one or more proteases expressed by a tissue to which the IFN protein is to be locally delivered.

158. The method of embodiment 157, wherein the IFN proprotein comprises one or more targeting moieties that recognize a target molecule expressed by the tissue.

159. The method of embodiment 158, wherein the IFN proprotein comprises two targeting moieties that each recognize a target molecule expressed by the tissue.

160. The method of embodiment 157, wherein the IFN proprotein comprises one or more means for binding a target molecule expressed by the tissue.

161. The method of embodiment 160, wherein the IFN proprotein comprises two means for binding a target molecule expressed by the tissue.

162. The method of any one of embodiments 157 to 161, wherein the tissue is cancer tissue.

163. The method of embodiment 162, wherein the target molecule expressed by the tissue is an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

164. The method of any one of embodiments 157 to 163, wherein an activated IFN protein comprising the IFN moiety is produced by cleavage of one or more protease-cleavable linkers in the IFN proprotein by one or more proteases in the tissue.

165. A method of treating cancer with an IFN protein that is selectively activated in cancer tissue, comprising administering to a subject in need thereof an IFN proprotein according to any one of embodiments 1 to 146 (or a pharmaceutical composition comprising the IFN proprotein and an excipient) which has one or more protease-cleavable linkers, each comprising one or more substrates for one or more proteases expressed by cancer tissue to which the IFN protein is targeted.

166. The method of embodiment 165, wherein the IFN proprotein comprises one or more targeting moieties that recognize a target molecule expressed by the cancer tissue or associated immune cells.

167. The method of embodiment 166, wherein the IFN proprotein comprises two targeting moieties that each recognize a target molecule expressed by the cancer tissue or associated immune cells.

168. The method of embodiment 165, wherein the IFN proprotein comprises one or more means for binding a target molecule expressed by the cancer tissue or associated immune cells.

169. The method of embodiment 168, wherein the IFN proprotein comprises two means for binding a target molecule expressed by the cancer tissue or associated immune cells.

170. The method of any one of embodiments 165 to 169, wherein the target molecule expressed by the cancer tissue or associated immune cells is an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

171. The method of any one of embodiments 165 to 170, wherein an activated IFN protein comprising the IFN moiety is produced by cleavage of one or more protease-cleavable linkers in the IFN proprotein by one or more proteases in the cancer tissue.

172. A method of administering to the subject IFN therapy with reduced systemic exposure and/or reduced systemic toxicity, comprising administering to a subject the IFN therapy in the form of an IFN proprotein according to any one of embodiments 1 to 146 (or a pharmaceutical composition comprising the IFN proprotein and an excipient) which has one or more protease-cleavable linkers, each comprising one or more substrates for one or more proteases expressed by a tissue for which IFN therapy is desirable and/or intended.

173. The method of embodiment 172, wherein the IFN proprotein comprises one or more targeting moieties that recognize a target molecule expressed by the tissue.

174. The method of embodiment 173, wherein the IFN proprotein comprises two targeting moieties that each recognize a target molecule expressed by the tissue.

175. The method of embodiment 172, wherein the IFN proprotein comprises one or more means for binding a target molecule expressed by the tissue.

176. The method of embodiment 175, wherein the IFN proprotein comprises two means for binding a target molecule expressed by the tissue.

177. The method of any one of embodiments 172 to 176, wherein the tissue is cancer tissue or associated immune cells.

178. The method of embodiment 177, wherein the target molecule expressed by the tissue is an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

179. The method of any one of embodiments 172 to 178, wherein an activated IFN protein comprising the IFN moiety is produced by cleavage of one or more protease-cleavable linkers in the IFN proprotein by one or more proteases in the tissue.

180. A method of treating cancer with an IFN protein that is selectively activated in cancer tissue, comprising administering to a subject in need thereof an IFN proprotein according to any one of embodiments 1 to 146 (or a pharmaceutical composition comprising the IFN proprotein and an excipient) which has one or more protease-cleavable linkers, each comprising one or more substrates for one or more proteases expressed by the cancer tissue.

181. The method of embodiment 180, wherein the IFN proprotein comprises one or more targeting moieties that recognize a target molecule expressed by the cancer tissue or associated immune cells.

182. The method of embodiment 181, wherein the IFN proprotein comprises two targeting moieties that each recognize a target molecule expressed by the cancer tissue or associated immune cells.

183. The method of embodiment 180, wherein the IFN proprotein comprises one or more means for binding a target molecule expressed by the cancer tissue or associated immune cells.

184. The method of embodiment 183, wherein the IFN proprotein comprises two means for binding a target molecule expressed by the cancer tissue or associated immune cells.

185. The method of any one of embodiments 180 to 184, wherein the target molecule expressed by the cancer tissue or associated immune cells is an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

186. The method of any one of embodiments 179 to 184, wherein an activated IFN protein comprising the IFN moiety is produced by cleavage of one or more protease-cleavable linkers in the IFN proprotein by one or more proteases in the cancer tissue.

187. A method of targeted delivery of an activated IFN protein to cancer tissue, comprising administering to a subject an IFN proprotein according to any one of embodiments 1 to 146 (or a pharmaceutical composition comprising the IFN proprotein and an excipient), wherein the IFN proprotein:
(a) comprises (i) one or more targeting moieties that recognize a target molecule expressed by the cancer tissue or associated immune cells, or (ii) means for binding a target molecule expressed by the cancer tissue or associated immune cells; and
(b) has one or more protease-cleavable linkers, each comprising one or more substrates for one or more proteases expressed in a tissue for which IFN therapy is desirable and/or intended.

188. The method of embodiment 187, wherein the IFN proprotein comprises (i) two targeting moieties that each recognize a target molecule expressed by the cancer tissue or associated immune cells, or (ii) two means for binding a target molecule expressed by the cancer tissue or associated immune cells.

189. The method of embodiment 187 or 188, wherein the target molecule expressed by the cancer tissue or associated immune cells is an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

190. The method of any one of embodiments 187 to 189, wherein an activated IFN protein comprising the IFN moiety is produced by cleavage of one or more protease-cleavable linkers in the IFN proprotein by one or more proteases in the cancer tissue.

191. A method of locally inducing an immune response in a target tissue, comprising administering to a subject an IFN proprotein according to any one of embodiments 1 to 146 (or a pharmaceutical composition comprising the IFN proprotein and an excipient) which has (i) one or more targeting moieties capable of binding a target molecule expressed in the target tissue or (ii) one or more means for binding a target molecule expressed in the target tissue and one or more protease-cleavable linkers, each protease-cleavable linker comprising one or more substrates for one or more proteases expressed in the target tissue.

192. The method of embodiment 191, wherein the IFN proprotein comprises (i) two targeting moieties that each recognize a target molecule expressed in the target tissue or associated immune cells or (ii) two means for binding a target molecule expressed in the target tissue or associated immune cells.

193. The method of embodiment 191 or 192, wherein the target tissue is cancer tissue.

194. The method of any one of embodiments 191 to 193, wherein the target molecule expressed in the target tissue or associated immune cells is an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

195. The method of any one of embodiments 190 to 194, wherein an activated IFN protein comprising the IFN moiety is produced by cleavage of one or more protease-cleavable linkers in the IFN proprotein by one or more proteases in the target tissue.

196. The method of embodiment 195, wherein the IFN protein induces the immune response against at least one cell type in the target tissue.

197. A method of enhancing an immune response against an antigen, comprising administering to a subject an immunogenic agent that elicits an immune response against the antigen together with an IFN proprotein (e.g., an IFN receptor agonist) according to any one of claims 1 to 146 (or a pharmaceutical composition comprising the IFN receptor agonist and an excipient) or a nucleic acid encoding such IFN proprotein (e.g., IFN receptor agonist), e.g., as described in Section 6.10.1).

198. The method of embodiment 197, wherein the administration of the immunogenic agent and the IFN proprotein (e.g., IFN receptor agonist) are concurrent, separate but simultaneous, or sequential.

199. The method of embodiment 197 or 198 or embodiment 1, wherein the immunogenic agent is a vaccine, optionally wherein the vaccine is a cancer vaccine or a vaccine against an infectious agent.

200. The method of any one of embodiments 151 to 199, wherein the administration is non-local.

201. The method of embodiment 200, wherein the administration is systemic.

### 8. EXAMPLES

### 8.1. Proprotein Construct Sequences

Table 3 below provides sequences of IFN proprotein and control constructs utilized in the studies described herein.

TABLE 3

| Construct | Sequence |
|---|---|
| Anti-PD1-PCL-IFN-PCL-Fc | Chain 1: |
| Chain 1: anti-hPD1-VH- | QVQLVQSGAEVKRPGSSVKVSCKVSGVTFRNFAIIWVRQAPGQGLE |
| CH1-CH2-CH3-PCL-IFN- | WMGGIIPFFSAANYAQSFQGRVTITPDESTSTAFMELASLRSEDTAVY |
| PCL-CH2-CH3 | YCAREGERGHTYGFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | PVAGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFY |
| | PSDIAVEWESNGQPENNYKTTPPVLDSDGSFFLYSRLTVDKSRWQE |
| | GNVFSCSVMHEALHNHYTQKSLSLSLGKISSGLLSGRSDNHCDLPQT |
| | HSLGSRRTLMLLAQMRRISLFSCLKDRHDFGFPQEEFGNQFQKAETI |
| | PVLHEMIQQIFNLFSTKDSSAAWDETLLDKFYTELYQQLNDLEACVIQ |
| | GVGVTETPLMKEDSILAVRKYFQRITLYLKEKKYSPCAWEVVRAEIMR |
| | SFSLSTNLQESLRSKEISSGLLSGRSDNHESKYGPPCPPCPAPPVAG |
| | PSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPS |
| | SIEKTISKAKGQPREPQVYTLPPSQEEMTKNQVSLTCLVKGFYPSDIA |
| | VEWESNGQPENNYKTTPPVLDSDGSFFLYSRLTVDKSRWQEGNVF |
| | SCSVMHEALHNHYTQKSLSLSLGK (SEQ ID NO: 385) |
| Chain 2: anti-hPD1-VH- | Chain 2: |
| CH1-CH2-CH3-PCL-IFN- | QVQLVQSGAEVKRPGSSVKVSCKVSGVTFRNFAIIWVRQAPGQGLE |
| PCL-CH2-CH3 | WMGGIIPFFSAANYAQSFQGRVTITPDESTSTAFMELASLRSEDTAVY |
| | YCAREGERGHTYGFDYWGQGTLVTVSS |
| | ASTKGPSVFPLAPCSRSTSESTAALGCLVKDYFPEPVTVSWNSGALT |
| | SGVHTFPAVLQSSGLYSLSSVVTVPSSSLGTKTYTCNVDHKPSNTKV |
| | DKRVESKYGPPCPPCPAPPVAGPSVFLFPPKPKDTLMISRTPEVTCV |
| | VVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGKI |
| | SSGLLSGRSDNHCDLPQTHSLGSRRTLMLLAQMRRISLFSCLKDRHD |
| | FGFPQEEFGNQFQKAETIPVLHEMIQQIFNLFSTKDSSAAWDETLLDK |
| | FYTELYQQLNDLEACVIQGVGVTETPLMKEDSILAVRKYFQRITLYLKE |
| | KKYSPCAWEVVRAEIMRSFSLSTNLQESLRSKEISSGLLSGRSDNHE |
| | SKYGPPCPPCPAPPVAGPSVFLFPPKPKDTLMISRTPEVTCVVVDVS |
| | QEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFL |
| | YSRLTVDKSRWQEGNVFSCSVMHEALHNHYTQKSLSLSLGK |
| | (SEQ ID NO: 385) |
| Chain 3: anti-hPD1 light | Chain 3: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Chain 4: anti-hPD1 light | Chain 4: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Anti-PD1-VH-CH1-PCL- | Chain 1: |
| IFN-PCL-Hinge-Fc | QVQLVQSGAEVKRPGSSVKVSCKVSGVTFRNFAIIWVRQAPGQGLE |
| Chain 1: Anti-PD1-VH- | WMGGIIPFFSAANYAQSFQGRVTITPDESTSTAFMELASLRSEDTAVY |
| CH1-PCL-IFN-PCL- | YCAREGERGHTYGFDYWGQGTLVTVSSASTKGPSVFPLAPCSRSTS |
| Hinge-CH2-CH3 | ESTAALGCLVKDYFPEPVTVSWNSGALTSGVHTFPAVLQSSGLYSLS |
| | SVVTVPSSSLGTKTYTCNVDHKPSNTKVDKRVESKYISSGLLSGRSD |
| | NHCDLPQTHSLGSRRTLMLLAQMRRISLFSCLKDRHDFGFPQEEFG |
| | NQFQKAETIPVLHEMIQQIFNLFSTKDSSAAWDETLLDKFYTELYQQL |
| | NDLEACVIQGVGVTETPLMKEDSILAVRKYFQRITLYLKEKKYSPCAW |
| | EVVRAEIMRSFSLSTNLQESLRSKEISSGLLSGRSDNHGPPCPPCPA |
| | PPVAGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSN |
| | KGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQ |
| | EGNVFSCSVMHEALHNHYTQKSLSLSLGK (SEQ ID NO: 387) |
| Chain 1: Anti-PD1-VH- | Chain 2: |
| CH1-PCL-IFN-PCL- | QVQLVQSGAEVKRPGSSVKVSCKVSGVTFRNFAIIWVRQAPGQGLE |
| Hinge-CH2-CH3 | WMGGIIPFFSAANYAQSFQGRVTITPDESTSTAFMELASLRSEDTAVY |
| | YCAREGERGHTYGFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSSLGTKTYTCNVDHKPSNTKVDKRVESKYISSGLLSGRSD |
| | NHCDLPQTHSLGSRRTLMLLAQMRRISLFSCLKDRHDFGFPQEEFG |
| | NQFQKAETIPVLHEMIQQIFNLFSTKDSSAAWDETLLDKFYTELYQQL |
| | NDLEACVIQGVGVTETPLMKEDSILAVRKYFQRITLYLKEKKYSPCAW |
| | EVVRAEIMRSFSLSTNLQESLRSKEISSGLLSGRSDNHGPPCPPCPA |
| | PPVAGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSN |
| | KGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQ |
| | EGNVFSCSVMHEALHNHYTQKSLSLSLGK (SEQ ID NO: 387) |
| Chain 3: anti-hPD1 light | Chain 3: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Chain 4: anti-hPD1 light | Chain 4: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Anti-PD1-VH-CH1-PCL- | Chain 1: |
| ΔN-IFN-ΔC-PCL-Hinge-Fc | QVQLVQSGAEVKRPGSSVKVSCKVSGVTFRNFAIIWVRQAPGQGLE |
| Chain 1: Anti-PD1-VH- | WMGGIIPFFSAANYAQSFQGRVTITPDESTSTAFMELASLRSEDTAVY |
| CH1-PCL-ΔN-IFN-ΔC- | YCAREGERGHTYGFDYWGQGTLVTVSSASTKGPSVFPLAPCSRSTS |
| PCL-Hinge-CH2-CH3 | ESTAALGCLVKDYFPEPVTVSWNSGALTSGVHTFPAVLQSSGLYSLS |
| | SVVTVPSSSLGTKTYTCNVDHKPSNTKVDKRVESKYISSGLLSGRSD |
| | NHTHSLGSRRTLMLLAQMRRISLFSCLKDRHDFGFPQEEFGNQFQK |
| | AETIPVLHEMIQQIFNLFSTKDSSAAWDETLLDKFYTELYQQLNDLEA |
| | CVIQGVGVTETPLMKEDSILAVRKYFQRITLYLKEKKYSPCAWEVVRA |
| | EIMRSFSLSTNLQISSGLLSGRSDNHGPPCPPCPAPPVAGPSVFLFP |
| | PKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWES |
| | NGQPENNYKTTPPVLDSDGSFFLYSRLTVDKSRWQEGNVFSCSVM |
| | HEALHNHYTQKSLSLSLGK (SEQ ID NO: 388) |
| Chain 2: Anti-PD1-VH- | Chain 2: |
| CH1-PCL-ΔN-IFN-ΔC- | QVQLVQSGAEVKRPGSSVKVSCKVSGVTFRNFAIIWVRQAPGQGLE |
| PCL-Hinge-CH2-CH3 | WMGGIIPFFSAANYAQSFQGRVTITPDESTSTAFMELASLRSEDTAVY |
| | YCAREGERGHTYGFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSSLGTKTYTCNVDHKPSNTKVDKRVESKYISSGLLSGRSD |
| | NHTHSLGSRRTLMLLAQMRRISLFSCLKDRHDFGFPQEEFGNQFQK |
| | AETIPVLHEMIQQIFNLFSTKDSSAAWDETLLDKFYTELYQQLNDLEA |
| | CVIQGVGVTETPLMKEDSILAVRKYFQRITLYLKEKKYSPCAWEVVRA |
| | EIMRSFSLSTNLQISSGLLSGRSDNHGPPCPPCPAPPVAGPSVFLFP |
| | PKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWES |
| | NGQPENNYKTTPPVLDSDGSFFLYSRLTVDKSRWQEGNVFSCSVM |
| | HEALHNHYTQKSLSLSLGK (SEQ ID NO: 388) |
| Chain 3: anti-hPD1 light | Chain 3: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Chain 4: anti-hPD1 light | Chain 4: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Anti-PD1-VH-CH1-PCL- | Chain 1: |
| IFN-ΔC-PCL-Hinge-Fc | QVQLVQSGAEVKRPGSSVKVSCKVSGVTFRNFAIIWVRQAPGQGLE |
| Chain 1: Anti-PD1-VH- | WMGGIIPFFSAANYAQSFQGRVTITPDESTSTAFMELASLRSEDTAVY |
| CH1-PCL-IFN-ΔC-PCL- | YCAREGERGHTYGFDYWGQGTLVTVSSASTKGPSVFPLAPCSRSTS |
| Hinge-CH2-CH3 | ESTAALGCLVKDYFPEPVTVSWNSGALTSGVHTFPAVLQSSGLYSLS |
| | SVVTVPSSSLGTKTYTCNVDHKPSNTKVDKRVESKYISSGLLSGRSD |
| | NHCDLPQTHSLGSRRTLMLLAQMRRISLFSCLKDRHDFGFPQEEFG |
| | NQFQKAETIPVLHEMIQQIFNLFSTKDSSAAWDETLLDKFYTELYQQL |
| | NDLEACVIQGVGVTETPLMKEDSILAVRKYFQRITLYLKEKKYSPCAW |
| | EVVRAEIMRSFSLSTNLQISSGLLSGRSDNHGPPCPPCPAPPVAGPS |
| | VFLFPPKPKDTLMISRTPEVTCVVVDVSQEDPEVQFNWYVDGVEVH |
| | NAKTKPREEQFNSTYRVVSVLTVLHQDWLNGKEYKCKVSNKGLPSSI |
| | EKTISKAKGQPREPQVYTLPPSQEEMTKNQVSLTCLVKGFYPSDIAV |
| | EWESNGQPENNYKTTPPVLDSDGSFFLYSRLTVDKSRWQEGNVFS |
| | CSVMHEALHNHYTQKSLSLSLGK (SEQ ID NO: 389) |
| Chain 2: Anti-PD1-VH- | Chain 2: |
| CH1-PCL-IFN-ΔC-PCL- | QVQLVQSGAEVKRPGSSVKVSCKVSGVTFRNFAIIWVRQAPGQGLE |
| Hinge-CH2-CH3 | WMGGIIPFFSAANYAQSFQGRVTITPDESTSTAFMELASLRSEDTAVY |
| | YCAREGERGHTYGFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSSLGTKTYTCNVDHKPSNTKVDKRVESKYISSGLLSGRSD |
| | NHCDLPQTHSLGSRRTLMLLAQMRRISLFSCLKDRHDFGFPQEEFG |
| | NQFQKAETIPVLHEMIQQIFNLFSTKDSSAAWDETLLDKFYTELYQQL |
| | NDLEACVIQGVGVTETPLMKEDSILAVRKYFQRITLYLKEKKYSPCAW |
| | EVVRAEIMRSFSLSTNLQISSGLLSGRSDNHGPPCPPCPAPPVAGPS |
| | VFLFPPKPKDTLMISRTPEVTCVVVDVSQEDPEVQFNWYVDGVEVH |
| | NAKTKPREEQFNSTYRVVSVLTVLHQDWLNGKEYKCKVSNKGLPSSI |
| | EKTISKAKGQPREPQVYTLPPSQEEMTKNQVSLTCLVKGFYPSDIAV |
| | EWESNGQPENNYKTTPPVLDSDGSFFLYSRLTVDKSRWQEGNVFS |
| | CSVMHEALHNHYTQKSLSLSLGK (SEQ ID NO: 389) |
| Chain 3: anti-hPD1 light | Chain 3: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Chain 4: anti-hPD1 light | Chain 4: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Anti-PD1-VH-CH1- | Chain 1: |
| LongHinge-PCL-IFN- | QVQLVQSGAEVKRPGSSVKVSCKVSGVTFRNFAIIWVRQAPGQGLE |
| PCL-LongHinge-Fc | WMGGIIPFFSAANYAQSFQGRVTITPDESTSTAFMELASLRSEDTAVY |
| Chain 1: Anti-PD1-VH- | YCAREGERGHTYGFDYWGQGTLVTVSS |
| CH1-LongHinge-PCL- | ASTKGPSVFPLAPCSRSTSESTAALGCLVKDYFPEPVTVSWNSGALT |
| IFN-PCL-LongHinge- | SGVHTFPAVLQSSGLYSLSSVVTVPSSSLGTKTYTCNVDHKPSNTKV |
| CH2-CH3 | DKRVESKYGPPCPPCPAPPVAISSGLLSGRSDNHCDLPQTHSLGSR |
| | RTLMLLAQMRRISLFSCLKDRHDFGFPQEEFGNQFQKAETIPVLHEMI |
| | QQIFNLFSTKDSSAAWDETLLDKFYTELYQQLNDLEACVIQGVGVTET |
| | PLMKEDSILAVRKYFQRITLYLKEKKYSPCAWEVVRAEIMRSFSLSTN |
| | LQESLRSKEISSGLLSGRSDNHESKYGPPCPPCPAPPVAGPSVFLFP |
| | PKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWES |
| | NGQPENNYKTTPPVLDSDGSFFLYSRLTVDKSRWQEGNVFSCSVM |
| | HEALHNHYTQKSLSLSLGK (SEQ ID NO: 390) |
| Chain 2: Anti-PD1-VH- | Chain 2: |
| CH1-LongHinge-PCL- | QVQLVQSGAEVKRPGSSVKVSCKVSGVTFRNFAIIWVRQAPGQGLE |
| IFN-PCL-LongHinge- | WMGGIIPFFSAANYAQSFQGRVTITPDESTSTAFMELASLRSEDTAVY |
| CH2-CH3 | YCAREGERGHTYGFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | PVAISSGLLSGRSDNHCDLPQTHSLGSRRTLMLLAQMRRISLFSCLK |
| | DRHDFGFPQEEFGNQFQKAETIPVLHEMIQQIFNLFSTKDSSAAWDE |
| | TLLDKFYTELYQQLNDLEACVIQGVGVTETPLMKEDSILAVRKYFQRIT |
| | LYLKEKKYSPCAWEVVRAEIMRSFSLSTNLQESLRSKEISSGLLSGRS |
| | DNHESKYGPPCPPCPAPPVAGPSVFLFPPKPKDTLMISRTPEVTCVV |
| | VDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQE |
| | EMTKNQVSLTCLVKGFYPSDIAVEWESNGQPENNYKTTPPVLDSDG |
| | SFFLYSRLTVDKSRWQEGNVFSCSVMHEALHNHYTQKSLSLSLGK |
| | (SEQ ID NO: 390) |
| Chain 3: anti-hPD1 light | Chain 3: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Chain 4: anti-hPD1 light | Chain 4: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Anti-PD1-VH-CH1- | Chain 1: |
| ShortHinge-PCL-IFN- | QVQLVQSGAEVKRPGSSVKVSCKVSGVTFRNFAIIWVRQAPGQGLE |
| PCL-ShortHinge-Fc | WMGGIIPFFSAANYAQSFQGRVTITPDESTSTAFMELASLRSEDTAVY |
| Chain 1: Anti-PD1-VH- | YCAREGERGHTYGFDYWGQGTLVTVSSASTKGPSVFPLAPCSRSTS |
| CH1-ShortHinge-PCL- | ESTAALGCLVKDYFPEPVTVSWNSGALTSGVHTFPAVLQSSGLYSLS |
| IFN-PCL-ShortHinge- | SVVTVPSSSLGTKTYTCNVDHKPSNTKVDKRVESKYGPPCPPCISSG |
| CH2-CH3 | LLSGRSDNHCDLPQTHSLGSRRTLMLLAQMRRISLFSCLKDRHDFGF |
| | PQEEFGNQFQKAETIPVLHEMIQQIFNLFSTKDSSAAWDETLLDKFYT |
| | ELYQQLNDLEACVIQGVGVTETPLMKEDSILAVRKYFQRITLYLKEKK |
| | YSPCAWEVVRAEIMRSFSLSTNLQESLRSKEISSGLLSGRSDNHCPP |
| | CPAPPVAGPSVFLFPPKPKDTLMISRTPEVTCVVVDVSQEDPEVQFN |
| | WYVDGVEVHNAKTKPREEQFNSTYRVVSVLTVLHQDWLNGKEYKC |
| | KVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKS |
| | RWQEGNVFSCSVMHEALHNHYTQKSLSLSLGK (SEQ ID NO: 391) |
| Chain 2: Anti-PD1-VH- | Chain 2: |
| CH1-ShortHinge-PCL- | QVQLVQSGAEVKRPGSSVKVSCKVSGVTFRNFAIIWVRQAPGQGLE |
| IFN-PCL-ShortHinge- | WMGGIIPFFSAANYAQSFQGRVTITPDESTSTAFMELASLRSEDTAVY |
| CH2-CH3 | YCAREGERGHTYGFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSSLGTKTYTCNVDHKPSNTKVDKRVESKYGPPCPPCISSG |
| | LLSGRSDNHCDLPQTHSLGSRRTLMLLAQMRRISLFSCLKDRHDFGF |
| | PQEEFGNQFQKAETIPVLHEMIQQIFNLFSTKDSSAAWDETLLDKFYT |
| | ELYQQLNDLEACVIQGVGVTETPLMKEDSILAVRKYFQRITLYLKEKK |
| | YSPCAWEVVRAEIMRSFSLSTNLQESLRSKEISSGLLSGRSDNHCPP |
| | CPAPPVAGPSVFLFPPKPKDTLMISRTPEVTCVWVDVSQEDPEVQFN |
| | WYVDGVEVHNAKTKPREEQFNSTYRVVSVLTVLHQDWLNGKEYKC |
| | KVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKS |
| | RWQEGNVFSCSVMHEALHNHYTQKSLSLSLGK (SEQ ID NO: 391) |
| Chain 3: anti-hPD1 light | Chain 3: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |
| Chain 4: anti-hPD1 light | Chain 4: |
| chain | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYSTP |
| | PITFGQGTRLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC* (SEQ ID NO: 386) |

### 8.2. Materials and Methods

### 8.2.1. Production of Type I IFN Constructs

Constructs encoding antibody and sterically attenuated IFN fusion proteins were generated in standard mammalian protein expression DNA vectors (pcDNA3.4 or similar) suitable for high yield protein production and containing standard elements such as promoter sequence, polyA sequence, regulatory elements, and resistance genes. Where applicable, sequences were codon-optimized. A 29-amino acid signal sequence from murine inactive tyrosine-protein kinase transmembrane receptor ROR1 (mROR1) was added to the N-termini of the constructs to serve as a signal for secretion. All IFN fusion proteins were expressed as preproteins containing the signal sequence which is cleaved by intracellular processing to produce a mature protein. The constructs were expressed in Expi293FTM cells by transient transfection (Thermo Fisher Scientific). Proteins in Expi293F supernatant were purified using the ProteinMaker system (Protein BioSolutions, Gaithersburg, MD) with either HiTrap™ Protein G HP or MabSelect SuRe pcc columns (Cytiva). After single step elution, the proteins were neutralized, dialyzed into a final buffer of phosphate buffered saline (PBS) with 5% glycerol, aliquoted and stored at −80 °C. Samples were further analyzed by SE-UPLC to determine the presence of high or low molecular weight species relative to the species of interest.

### 8.2.2. In Vitro Cleavage of IFN Constructs

The protease-cleavable linkers were cleaved enzymatically either by incubating the proprotein constructs with uPA or MMP enzymes. For enzymatic cleavage with uPA, 8 μg of protein constructs were incubated with 100 ng of uPA enzyme in 200 μL volume of uPA buffer (50 mM Tris pH8.5, 0.01% (v/v) Tween20) for 20 hours at 37 °C. For enzymatic cleavage with MMP, 8 μg of protein constructs were incubated with 200 MMP2 and MMP9 each, in 200 μL volume of MMP buffer (50 mM Tris pH7.5, 150 mM NaCl, 10 mM CaCl₂, 0.05% Brij35) for 20 hours at 37 °C.

### 8.2.3. Engineering of Reporter KG-1a Cells

The promyeloblast macrophage cell line KG-1a was transduced with an ISRE-driven luciferase reporter construct and maintained in Iscove's modified Dulbecco's medium supplemented with 2 mM L-Glutamine/Penicillin/Streptomycin+20% FBS+1 μg/mL puromycin. A single cell clone, having high responsiveness to IFNα2b, was identified and renamed KG-1a/ISRE-Luc cl.2F5, and was used for assays as noted.

### 8.2.4. Luciferase Assay Setup

RPMI1640 media supplemented with 2 mM L-Glutamine/Penicillin/Streptomycin+10% FBS was used as assay medium to prepare cell suspensions and fusion protein dilutions.

The day of the assay, cells were centrifuged and resuspended in assay medium at a density of 5×10⁵/mL. IFNα2b and or IFN fusion proteins were diluted 1:5 following a 11-point dilution range (100 nM to 10.2fM range for recombinant Interferons and 500 nM to 51.2fM range for fusion proteins), with the 12th point containing no recombinant protein. 2.5×10⁴ reporter cells were added to 96-well white flat bottom plates and incubated with serially diluted recombinant IFN or IFN fusion protein. Plates were incubated for 5 hours at 37 °C and 5% CO₂, before the addition of 100 μL ONE-Glo™ (Promega) reagent to lyse cells and detect luciferase activity. The emitted light was captured in relative light units (RLU) on a multilabel plate reader Envision (PerkinElmer).

### 8.3. Example 1: SE-UPLC Profiles of Fc-Linked Interferon Molecules

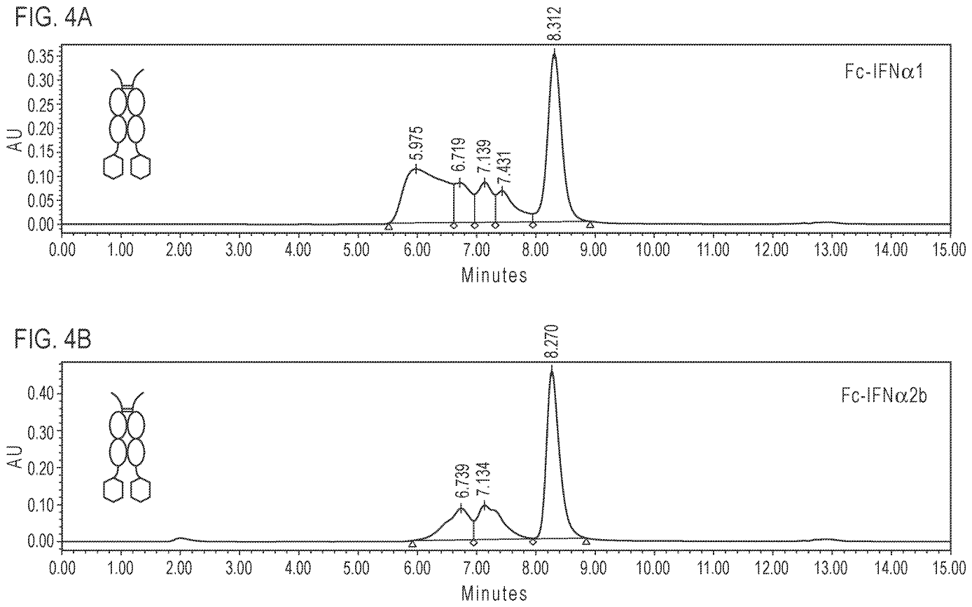

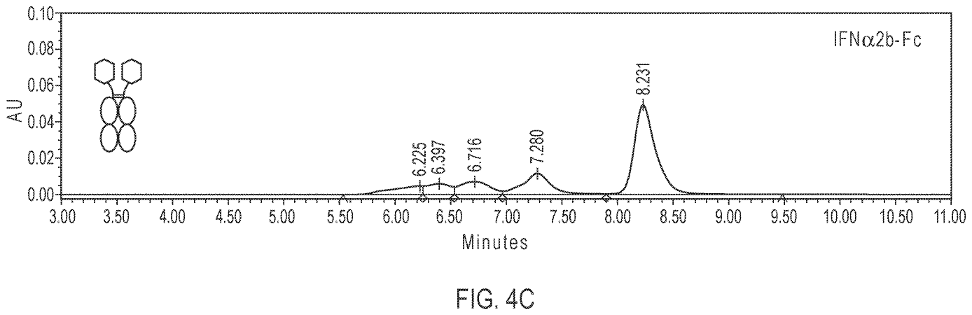

SE-UPLC was conducted to assess the IFN molecules that are linked to Fc domains either on the C-terminus or on the N-terminus. The three exemplary constructs analyzed with SE-UPLC, Fc-IFNα1 (FIG. 4A), Fc-IFNα2b (FIG. 4B), and IFNα2b-Fc (FIG. 4C), displayed discrete main peaks with varying levels of high molecular weight species. The main peak percent area of Fc-IFNα1 was calculated to be 37.43, whereas these percentage values were larger for the Fc-IFNα2b and IFNα2b-Fc, which were calculated to be 57.66 and 56.4, respectively.

### 8.4. Example 2: Activity of Interferon Molecules

An Interferon-Stimulated Response Element (ISRE) driven luciferase reporter was incorporated into the promyeloblast macrophage cell line KG-1a as described in Section 8.2.3 and was used as described in Section 8.2.4 to evaluate ability of IFN receptor agonist constructs to induce ISRE.

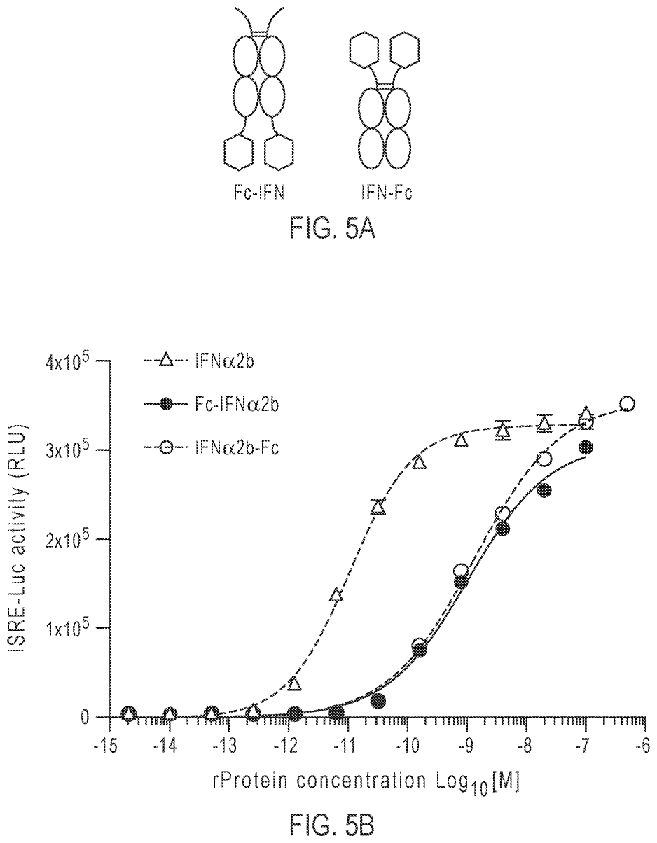

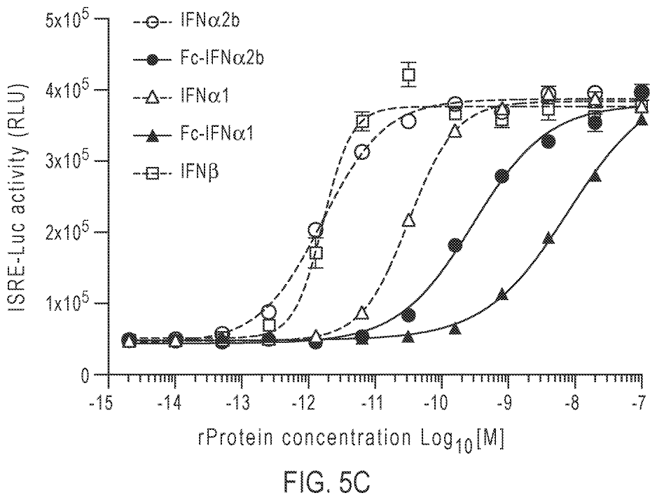

The results, shown in FIG. 5, indicated that recombinant protein and Fc fusions of IFN variants show varying degrees of attenuation in an in vitro luciferase assay of interferon-sensitive response element (ISRE). The first assessment involved testing whether there were differences in activity in IFN molecules that are linked to an Fc molecule either on the N- or the C-terminus (FIG. 5A). Relative to IFNα2b, both Fc-IFNα2b, and IFNα2b-Fc displayed weaker interferon signaling. However, the level of attenuation was similar for both molecules (FIG. 5B). Next, in vitro activity of two Fc-IFN constructs, Fc-IFNα2b and Fc-IFNα1, were compared to the activity of three IFN variants, IFNα2b, IFNα1, and IFNβ (FIG. 5C). Among the IFN variants, the highest level of activity was observed with IFNβ and IFNα2b, whereas the activity of IFNα1 was relatively weaker. Recombinant proteins with Fc fusions displayed an attenuated level of activity compared to the IFN variants. In conclusion, Fc fusion leads to attenuation of interferon signaling relative to free interferon.

### 8.5. Example 3: SE-UPLC Profiles of Mutant IFN Constructs

SE-UPLC was conducted to assess mutant IFN molecules that are linked to Fc domains on the C-terminus. The four exemplary constructs analyzed with SE-UPLC, Fc-IFNα2bR33A (FIG. 6A), Fc-IFNα2bR149A (FIG. 6B), Fc-IFNα2bR120A (FIG. 6C), and Fc-IFNα2bS152A (FIG. 6D), displayed discrete main peaks with varying levels of high molecular weight species.

### 8.6. Example 4: Activity of Mutant IFN Constructs

The ISRE-driven luciferase reporter assay was incorporated into the promyeloblast macrophage cell line KG-1a as described in Section 8.2.3 and was used as described in Section 8.2.4 to evaluate ability of mutant IFN receptor agonist constructs to induce ISRE.

Activity of IFN variants correlates with their affinity to IFNAR. Hence, mutations that affect the IFN-IFNAR binding can influence the activity of Fc-IFN constructs. A series of mutations were introduced to IFNα2b either on its IFNAR1 or IFNAR2 interface (FIGS. 7A and 7B). Relative to wild-type Fc-IFNα2b, most mutations that interfere with IFNAR1 or IFNAR2 binding of Fc-IFNα2b attenuated the ISRE-luciferase activity. Moreover, the degree of this attenuation varied; whereas some mutations caused only a slight attenuation of activity, others led to very high levels of attenuation. Nonetheless, there was minimal distinction between mutations that interfered with IFNAR1 or IFNAR2 binding for these differences in attenuation levels.

### 8.7. Example 5: SE-UPLC Profiles of Exemplary Interferon Proprotein Constructs

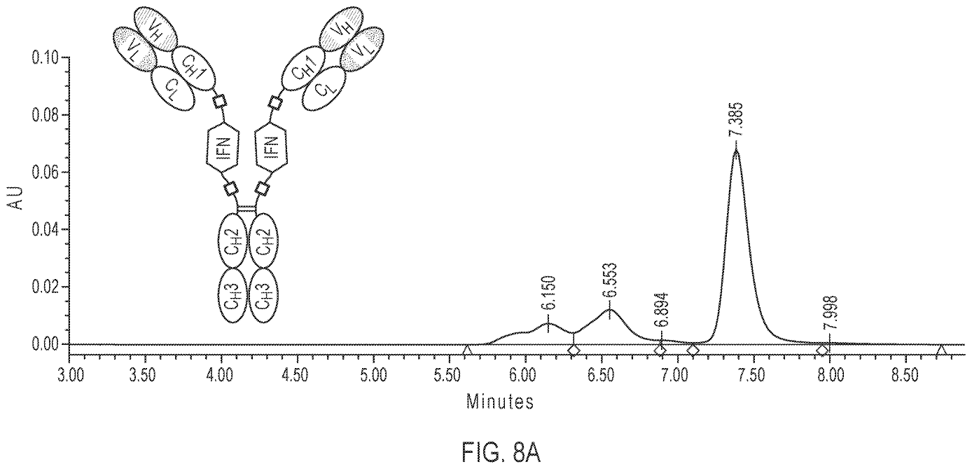

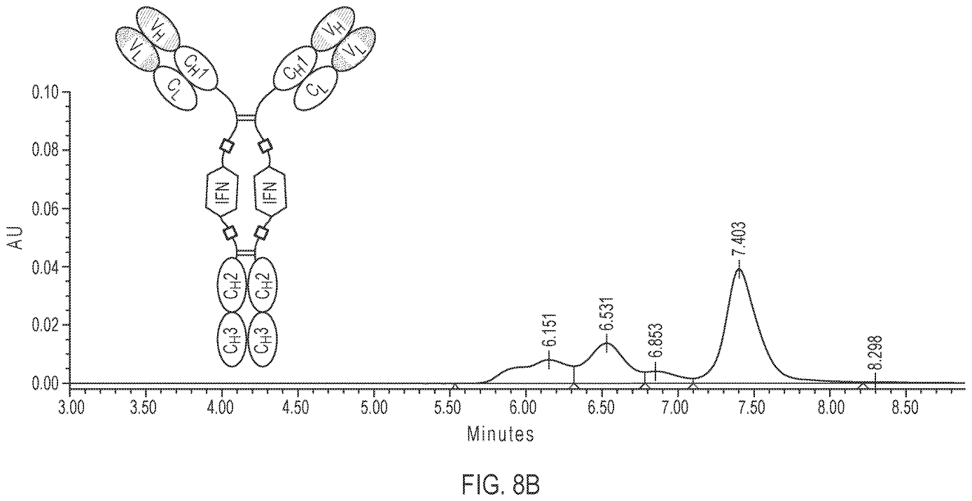

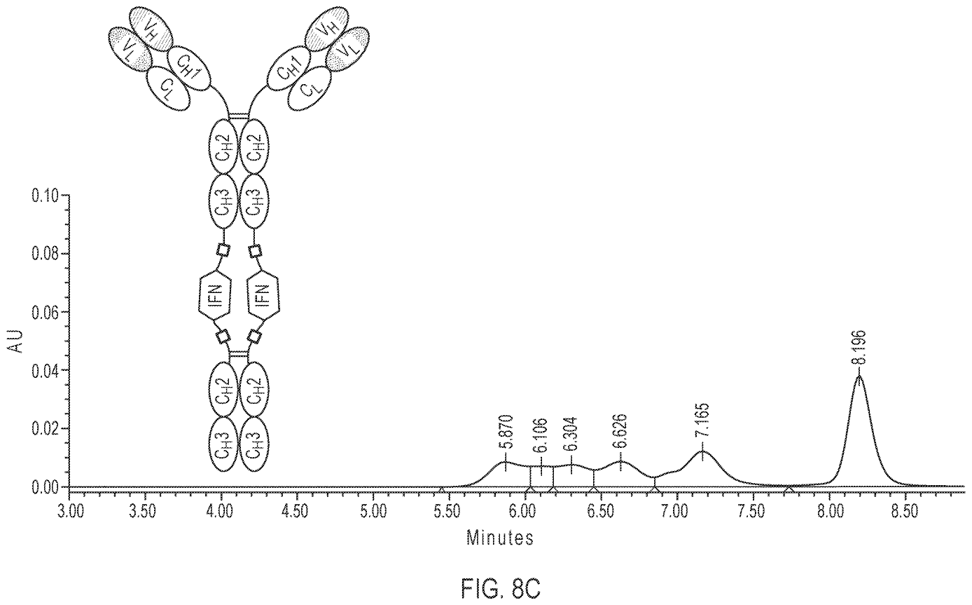

After constructs encoding sterically attenuated IFN fusion proteins were generated, SE-UPLC was conducted to assess the presence of high or low molecular weight species in samples as described in Section 8.2.1. FIG. 8 illustrates the profiles of three exemplary IFN proprotein constructs: a single hinge IFN construct (FIG. 8A), a double hinge IFN construct (FIG. 8B), and an IFN construct with two Fc domains (FIG. 8C). Whereas the single and double hinge constructs displayed discrete main peaks, the SEC profile of the IFN construct with two Fc domains was less clear. Therefore, subsequent analyses were conducted with single and double hinge IFN constructs and excluded the IFN constructs with two Fc domains.

### 8.8. Example 6: Enzymatic Cleavage of IFN Proprotein Constructs

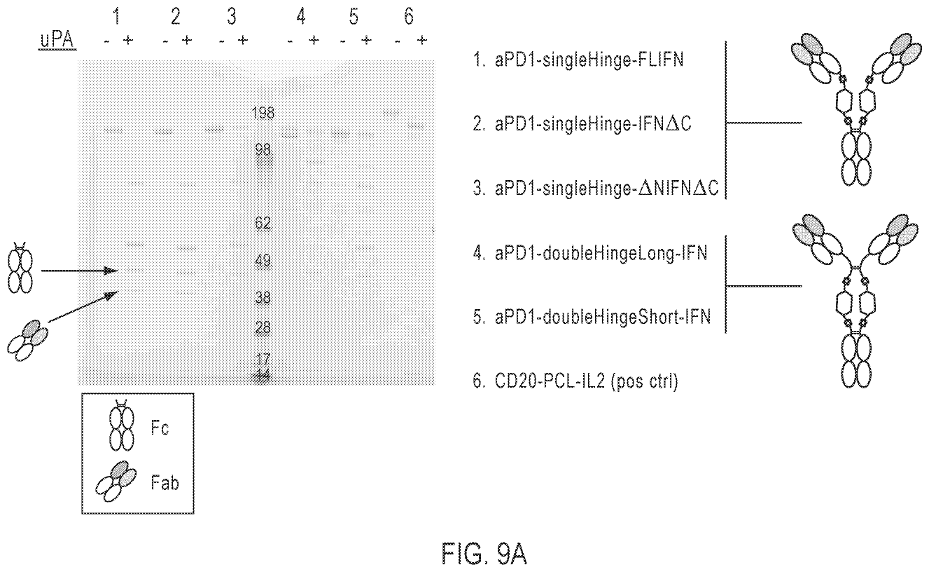

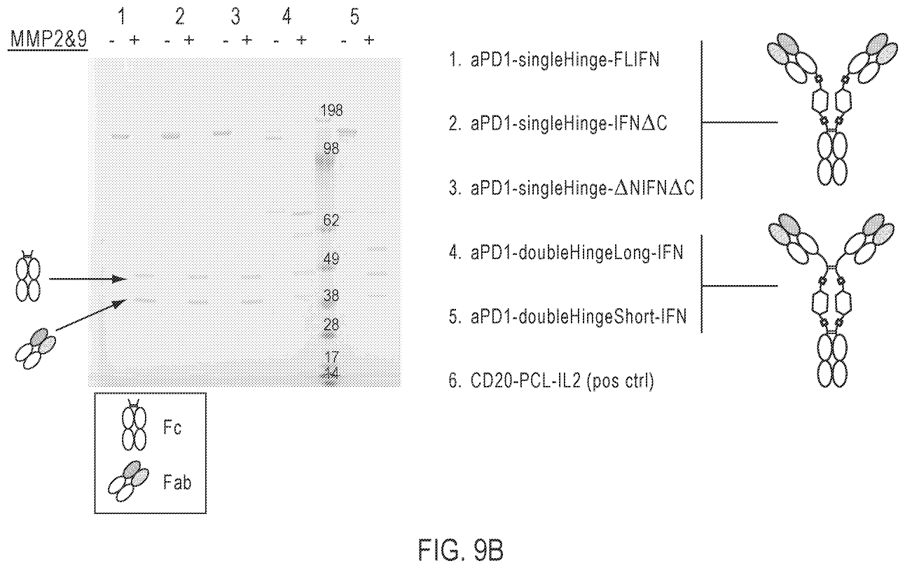

Exemplary IFN proprotein constructs were cleaved with uPA and MMP enzymes as described in Section 8.2.2. (FIG. 9). Cleavage of the proprotein constructs was assessed by the presence of bands that correspond to Fc and Fab components both of which were expected to appear between 38 and 49 kDa. Identity of different fragments was further confirmed by Western blot detecting the Fc portion.

Cleavage of both single hinge and double hinge IFN proprotein constructs by uPA resulted in additional bands in an SDS-PAGE image that appeared at higher molecular weights than the expected bands (FIG. 9A), indicating incomplete cleavage of these constructs by uPA in vitro. In contrast, MMP2/9 completely cleaved single hinge IFN proproteins. However, in the lanes loaded with MMP2/9-cleaved double hinge IFN proproteins, there were additional high molecular weight bands (FIG. 9B), indicating incomplete MMP cleavage of double hinge constructs.

### 8.9. Example 7: Activity of IFN Proprotein Constructs

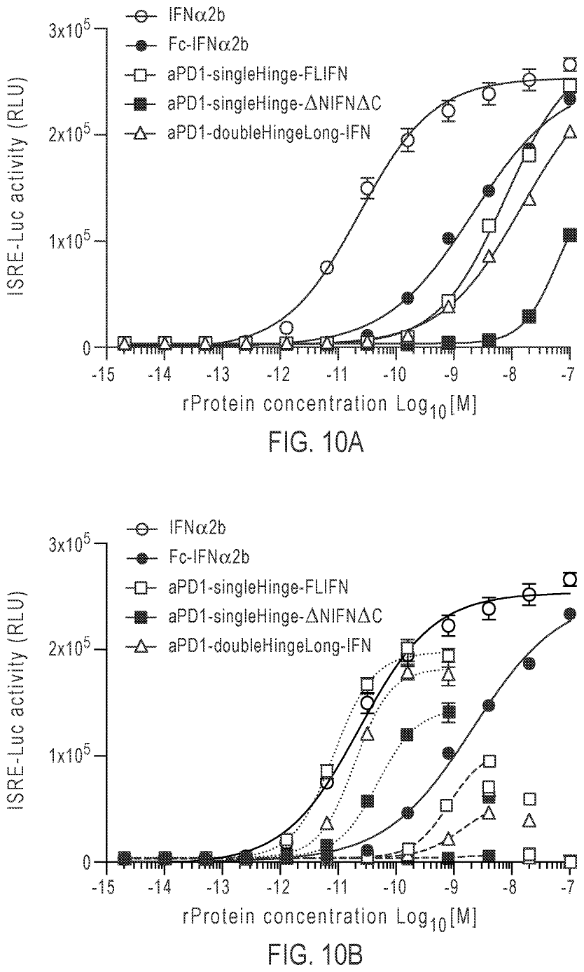

The ISRE-luciferase activity of three IFN proproteins was assessed in comparison to IFNα2b and Fc-IFNα2b. The single hinge full length IFN and the double hinge IFN constructs showed modest levels of attenuation relative to Fc-IFNα2b. However, the best attenuation was associated with the single hinge truncated IFN (FIG. 10A).

To assess whether in vitro cleavage of IFN proprotein constructs restores the activity of IFN, the same three IFN proprotein constructs were first incubated with either MMP buffer control or MMP2 and MMP9 enzymes in MMP buffer as described in Section 8.2.2. The constructs that were incubated only with the MMP buffer attenuated the activity of IFN proprotein constructs (FIG. 10B, dashed lines) which resembled the attenuation of activity by the noncleaved constructs seen in FIG. 10A. The presence of MMP buffer was associated with a general reduction of bioassay signal. Yet, when the constructs were incubated with the MMP enzymes in the same buffer, the release of IFN was associated with increased potency of activity relative to the noncleaved constructs (FIG. 10B, dotted lines). More specifically, the activity of the single hinge full length IFN and the double hinge IFN constructs were similar to that of IFNα2b, whereas the activity of the single hinge truncated IFN was between those of IFNα2b and Fc-IFNα2b.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A Type I interferon (IFN) proprotein, comprising
(a) a first polypeptide chain comprising:
(i) a first immunoglobulin constant domain;
(ii) a first linker;
(iii) a first Type I interferon (IFN) moiety;
(iv) a second linker; and
(v) a first Fc domain;
(b) a second polypeptide chain comprising:
(i) a second immunoglobulin constant domain;
(ii) a third linker;
(iii) a second Type I interferon (IFN) moiety;
(iv) a fourth linker; and
(v) a second Fc domain associated with the first Fc domain to form an Fc region;
wherein at least two of the first linker, second linker, third linker and fourth linker are protease-cleavable linkers (PCLs), optionally wherein the IFN moieties in the IFN proprotein are sterically hindered from binding to an IFN receptor by the Fc domains.

2. The IFN proprotein of claim 1, wherein the first and second IFN moieties each comprise an amino acid sequence having at least about 90%, at least about 95%, or at least about 98% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.

3. The IFN proprotein of claim 1, wherein the first and second IFN moieties each comprise an amino acid sequence having one or more attenuating mutations as compared to mature human IFNα1 or IFNα2b.

4. The IFN proprotein of any one of claims 1 to 3, wherein the first and second IFN moieties each comprise one or more mutations selected from L26A, F27A, R33A, R33K, L30A, D35E, H57Y, E58N, Q61S, H57S, E58S, H57A, E58A, Q61A, Q90A, E96A, R120A, L135A, R144A, R144S, R144T, R144Y, R1441, R144L, A145D, A145H, A145K, A145M, A145V, A145Y, R149A, R149K, S152A, R162A, and E165D.

5. The IFN proprotein of any one of claims 1 to 4, wherein the first linker, second linker, third linker and fourth linker are protease-cleavable linkers (PCLs).

6. The IFN proprotein of any one of claims 1 to 4, wherein the first linker and third linker are protease-cleavable linkers (PCLs), optionally wherein the second linker and fourth linker are non-cleavable linkers (NCLs).

7. The IFN proprotein of any one of claims 1 to 4, wherein the second linker and fourth linker are protease-cleavable linkers (PCLs), optionally wherein the first linker and third linker are non-cleavable linkers (NCLs).

8. The IFN proprotein of any one of claims 1 to 7, wherein the PCLs each comprise a substrate sequence cleavable by any protease set forth in Table A.

9. The IFN proprotein of any one of claims 1 to 8, wherein the PCLs each comprise one or more substrate sequences selected from the substate sequences set forth in Table B.

10. The IFN proprotein of any one of claims 1 to 9, wherein the PCLs each comprise one or more spacer sequences selected from the sequences set forth in Table C.

11. The IFN proprotein of any one of claims 1 to 10, wherein the PCLs each comprise the amino acid sequence of any of the PCL sequences set forth in Table D or a variant thereof with up to 5 amino acid substitutions.

12. The IFN proprotein of any one of claims 1 to 11, wherein the first and third linkers are identical and/or the third and fourth linkers are identical.

13. The IFN proprotein of any one of claims 1 to 12, wherein the first Fc domain and/or the second Fc domain comprises a hinge domain.

14. The IFN proprotein of any one of claims 1 to 13, which further comprises a first targeting moiety and a second targeting moiety.

15. The IFN proprotein of claim 14, wherein the first targeting moiety and second targeting moiety are antibodies or antigen-binding fragments thereof.

16. The IFN proprotein of claim 15, wherein the first targeting moiety and second targeting moiety comprise Fab domains.

17. The IFN proprotein of any one of claims 14 to 16, wherein the first and second targeting moieties are N-terminal to the first and third linkers, respectively.

18. The IFN proprotein of any one of claims 14 to 17, wherein the first immunoglobulin constant domain is part of the first targeting moiety and the second immunoglobulin constant domain is part of the second targeting moiety.

19. The IFN proprotein of claim 18, wherein the first immunoglobulin constant domain is a CH3 domain or a CH1 domain.

20. The IFN proprotein of any one of claims 1 to 19, which is configured as illustrated in FIG. 1A, FIG. 2A or FIG. 2D.

21. The IFN proprotein of any one of claims 1 to 19, which is configured as illustrated in FIG. 1B, FIG. 2B or FIG. 2E.

22. The IFN proprotein of any one of claims 1 to 19, which is configured as illustrated in FIG. 1C, FIG. 2C or FIG. 2F.

23. The IFN proprotein of any one of claims 14 to 22, herein the first targeting moiety and/or second targeting moiety is capable of binding to an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

24. The IFN proprotein of any one of claims 14 to 23, wherein the first targeting moiety and/or second targeting moiety (a) comprises the (i) CDR or (ii) VH and VL sequences of antibody set forth in Table F or (b) competes with the antibody set forth in Table F for binding to the target molecule.

25. The IFN proprotein of any one of claims 14 to 23, wherein the first targeting moiety and/or second targeting moiety is capable of binding to an ECM antigen which is optionally selected from syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

26. The IFN proprotein of any one of claims 14 to 23, wherein the first targeting moiety and/or second targeting moiety is capable of binding to a cell surface molecule of tumor or viral lymphocytes.

27. The IFN proprotein of claim 26, wherein the antigen is a T-cell co-stimulatory protein, optionally selected from CD27, CD28, 4-1BB (CD137), OX40, CD30, CD40, ICOS, lymphocyte function-associated antigen-1 (LFA-1), CD2, CD7, LIGHT, NKG2C, and B7-H3.

28. The IFN proprotein of any one of claims 14 to 23, wherein the first targeting moiety and/or second targeting moiety is capable of binding to a checkpoint inhibitor.

29. The IFN proprotein of claim 28, wherein the checkpoint inhibitor is PDL1.

30. The IFN proprotein of claim 28, wherein the checkpoint inhibitor is PD1.

31. The IFN proprotein of any one of claims 14 to 23, wherein the first targeting moiety and/or second targeting moiety is capable of binding to a tumor-associated antigen (TAA), optionally selected from AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvlll, ErbB2/Her2, ErbB3, ErbB4, ETV6-AML, EpCAM, EphA2, Fra-1, FOLR1, a GAGE protein (e.g., GAGE-1 or-2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

32. The IFN proprotein of any one of claims 14 to 23, wherein the first targeting moiety and/or second targeting moiety is capable of binding to a dendritic cell (DC) antigen which is optionally selected from XCR1, Clec9a, CD1c, CD11c, CD14, PDL1, macrophage mannose receptor (CD206), and DEC-205.

33. The IFN proprotein of any one of claims 14 to 23, wherein the first targeting moiety and/or second targeting moiety is capable of binding to a natural killer (NK) cell antigen.

34. The IFN proprotein of any one of claims 1 to 33, wherein the Fc region is homodimeric.

35. A nucleic acid or plurality of nucleic acids encoding the IFN proprotein of any one of claims 1 to 34.

36. A host cell engineered to express the IFN proprotein of any one of claims 1 to 34 or the nucleic acid(s) of claim 35.

37. A method of producing the IFN proprotein of any one of claims 1 to 34, comprising culturing the host cell of claim 36 and recovering the IFN proprotein expressed thereby.

38. A pharmaceutical composition comprising the IFN proprotein of any one of claims 1 to 34 and an excipient.

39. A method of treating cancer, comprising administering to a subject in need thereof the IFN proprotein of any one of claims 1 to 34 or the pharmaceutical composition of claim 38.

40. The method of claim 39, wherein the IFN proprotein comprises at least one targeting moiety that is capable of binding to a target molecule.

41. The method of claim 39, wherein the cancer is associated with expression of the target molecule, e.g., a TAA and associated cancer as set forth in Table I.

42. The method of any one of claims 39 to 41, wherein an activated IFN protein comprising the IFN moiety is produced by cleavage of one or more protease-cleavable linkers in the IFN proprotein by one or more proteases expressed by the cancer tissue.

43. A method of localized delivery of an IFN protein, comprising administering to a subject an IFN proprotein according to any one of claims 1 to 34 (or a pharmaceutical composition comprising the IFN proprotein and an excipient) which has one or more protease-cleavable linkers, each comprising one or more substrates for one or more proteases expressed by a tissue to which the IFN protein is to be locally delivered.

44. A method of treating cancer with an IFN protein that is selectively activated in cancer tissue, comprising administering to a subject in need thereof an IFN proprotein according to any one of claims 1 to 34 (or a pharmaceutical composition comprising the IFN proprotein and an excipient) which has one or more protease-cleavable linkers, each comprising one or more substrates for one or more proteases expressed by cancer tissue to which the IFN protein is targeted.

45. A method of administering to the subject IFN therapy with reduced systemic exposure and/or reduced systemic toxicity, comprising administering to a subject the IFN therapy in the form of an IFN proprotein according to any one of claims 1 to 34 (or a pharmaceutical composition comprising the IFN proprotein and an excipient) which has one or more protease-cleavable linkers, each comprising one or more substrates for one or more proteases expressed by a tissue for which IFN therapy is desirable and/or intended.

46. A method of treating cancer with an IFN protein that is selectively activated in cancer tissue, comprising administering to a subject in need thereof an IFN proprotein according to any one of claims 1 to 34 (or a pharmaceutical composition comprising the IFN proprotein and an excipient) which has one or more protease-cleavable linkers, each comprising one or more substrates for one or more proteases expressed by the cancer tissue.

47. A method of targeted delivery of an activated IFN protein to cancer tissue, comprising administering to a subject an IFN proprotein according to any one of claims 1 to 34 (or a pharmaceutical composition comprising the IFN proprotein and an excipient), wherein the IFN proprotein:
(a) comprises one or more targeting moieties that recognize a target molecule expressed by the cancer tissue or associated immune cells; and
(b) has one or more protease-cleavable linkers, each comprising one or more substrates for one or more proteases expressed in a tissue for which IFN therapy is desirable and/or intended.

48. A method of locally inducing an immune response in a target tissue, comprising administering to a subject an IFN proprotein according to any one of claims 1 to 34 (or a pharmaceutical composition comprising the IFN proprotein and an excipient) which has one or more targeting moieties capable of binding a target molecule expressed in the target tissue and one or more protease-cleavable linkers, each comprising one or more substrates for one or more proteases expressed in the target tissue.

49. A method of enhancing an immune response against an antigen, comprising administering to a subject an immunogenic agent that elicits an immune response against the antigen together with an IFN proprotein according to any one of claims 1 to 34 (or a pharmaceutical composition comprising the IFN proprotein and an excipient) or a nucleic acid encoding such IFN proprotein, e.g., as described in Section 6.10.1).

50. The method of any one of claims 39 to 49, wherein the administration is non-local.


SEQUENCE LISTING (396 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 166 aa, synthetic construct
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 4 - protein, 166 aa, synthetic construct
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNVDSILAVKKYFRRITLYLTEKKYSPCAWEVVRAEIMRSLSLSTNLQERLRRKE

SEQ ID NO: 5 - protein, 165 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 6 - protein, 165 aa, synthetic construct [sequence omitted]

SEQ ID NO: 7 - protein, 165 aa, synthetic construct
CDLPQTHSLGSRRTLMLLAQMRRISLFSCLKDAHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 8 - protein, 165 aa, synthetic construct
CDLPQTHSLGSRRTLMLLAQMRRISLFSCLKDKHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 9 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYAQLNDLEACVIQGVGVTETPLMKEDSILAVRKYFQRITLYLKEKKYSPCAWEVVRAEIMRSFSLSTNLQESLRSKE

SEQ ID NO: 10 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLAACVIQGVGVTETPLMKEDSILAVRKYFQRITLYLKEKKYSPCAWEVVRAEIMRSFSLSTNLQESLRSKE

SEQ ID NO: 11 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVAKYFQRITLYLKEKKYSPCAWEVVRAEIMRSFSLSTNLQESLRSKE

SEQ ID NO: 12 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRMEIMRSFSLSTNLQESLRSKE

SEQ ID NO: 13 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 14 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMKSFSLSTNLQESLRSKE

SEQ ID NO: 15 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFALSTNLQESLRSKE

SEQ ID NO: 16 - protein, 153 aa, synthetic construct
THSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQ

SEQ ID NO: 17 - protein, 158 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQ

SEQ ID NO: 18 - protein, 19 aa, synthetic construct
YNLLGFLQRSSNFQCQKLL

SEQ ID NO: 19 - protein, 19 aa, synthetic construct
KEDAALTIYEMLQNIFAIF

SEQ ID NO: 20 - protein, 26 aa, synthetic construct
ETIVENLLANVYHQINHLKTVLEEKL

SEQ ID NO: 21 - protein, 18 aa, synthetic construct
SSLHLKRYYGRILHYLKA

SEQ ID NO: 22 - protein, 22 aa, synthetic construct
HCAWTIVRVEILRNFYFINRLT

SEQ ID NO: 23 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 24 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 25 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 26 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDKNENEDMKEMKENEMKPSEARVPQLSSLELRRYFHRIDNFLKEKKYSDCAWEIVRVEIRRCLYYFYKFTALFRRK

SEQ ID NO: 27 - protein, 40 aa, synthetic construct
DEDEDEDEDEDEDEDERPLALWRSDRDRDRDRDRDRDRDR

SEQ ID NO: 28 - protein, 8 aa, synthetic construct
AARGPAIH

SEQ ID NO: 29 - protein, 8 aa, synthetic construct
AAYHLVSQ

SEQ ID NO: 30 - protein, 8 aa, synthetic construct
AGLGISST

SEQ ID NO: 31 - protein, 8 aa, synthetic construct
AGLGVVER

SEQ ID NO: 32 - protein, 4 aa, synthetic construct
ALAL

SEQ ID NO: 33 - protein, 8 aa, synthetic construct
ALFFSSPP

SEQ ID NO: 34 - protein, 8 aa, synthetic construct
ALFKSSFP

SEQ ID NO: 35 - protein, 7 aa, synthetic construct
ALLLALL

SEQ ID NO: 36 - protein, 8 aa, synthetic construct
AQFVLTEG

SEQ ID NO: 37 - protein, 8 aa, synthetic construct
AQNLLGMV

SEQ ID NO: 38 - protein, 8 aa, synthetic construct
AVGLLAPP

SEQ ID NO: 39 - protein, 4 aa, synthetic construct
DAFK

SEQ ID NO: 40 - protein, 4 aa, synthetic construct
DEVD

SEQ ID NO: 41 - protein, 5 aa, synthetic construct
DEVDP

SEQ ID NO: 42 - protein, 6 aa, synthetic construct
DPRSFL

SEQ ID NO: 43 - protein, 8 aa, synthetic construct
DVAQFVLT

SEQ ID NO: 44 - protein, 4 aa, synthetic construct
DVLK

SEQ ID NO: 45 - protein, 8 aa, synthetic construct
DWLYWPGI

SEQ ID NO: 46 - protein, 7 aa, synthetic construct
EDDDDKA

SEQ ID NO: 47 - protein, 6 aa, synthetic construct
  SITE 3: note = Citrulline
  SITE 5: note = Hypofluorous acid modified tyrosine
EPXGYL

SEQ ID NO: 48 - protein, 8 aa, synthetic construct
EPQALAMS

SEQ ID NO: 49 - protein, 8 aa, synthetic construct
ESLPVVAV

SEQ ID NO: 50 - protein, 8 aa, synthetic construct
ESPAYYTA

SEQ ID NO: 51 - protein, 10 aa, synthetic construct
FPRPLGITGL

SEQ ID NO: 52 - protein, 8 aa, synthetic construct
FRLLDWQW

SEQ ID NO: 53 - protein, 4 aa, synthetic construct
GFLG

SEQ ID NO: 54 - protein, 10 aa, synthetic construct
GGAANLVRGG

SEQ ID NO: 55 - protein, 5 aa, synthetic construct
GGGRR

SEQ ID NO: 56 - protein, 8 aa, synthetic construct
GGPRGLPG

SEQ ID NO: 57 - protein, 10 aa, synthetic construct
GGQPSGMWGW

SEQ ID NO: 58 - protein, 7 aa, synthetic construct
GGSIDGR

SEQ ID NO: 59 - protein, 8 aa, synthetic construct
GGWHTGRN

SEQ ID NO: 60 - protein, 5 aa, synthetic construct
GIAGQ

SEQ ID NO: 61 - protein, 6 aa, synthetic construct
GKAFRR

SEQ ID NO: 62 - protein, 8 aa, synthetic construct
GPAGLYAQ

SEQ ID NO: 63 - protein, 8 aa, synthetic construct
GPAGMKGL

SEQ ID NO: 64 - protein, 8 aa, synthetic construct
GPEGLRVG

SEQ ID NO: 65 - protein, 8 aa, synthetic construct
GPLGIAGI

SEQ ID NO: 66 - protein, 7 aa, synthetic construct
GPLGVRG

SEQ ID NO: 67 - protein, 8 aa, synthetic construct
GPQGIAGQ

SEQ ID NO: 68 - protein, 8 aa, synthetic construct
GPQGLLGA

SEQ ID NO: 69 - protein, 6 aa, synthetic construct
GPRSFG

SEQ ID NO: 70 - protein, 7 aa, synthetic construct
GPRSFGL

SEQ ID NO: 71 - protein, 8 aa, synthetic construct
GPSHLVLT

SEQ ID NO: 72 - protein, 10 aa, synthetic construct
GVSQNYPIVG

SEQ ID NO: 73 - protein, 10 aa, synthetic construct
GVVQASCRLA

SEQ ID NO: 74 - protein, 6 aa, synthetic construct
GWEHDG

SEQ ID NO: 75 - protein, 6 aa, synthetic construct
HSSKLQ

SEQ ID NO: 76 - protein, 9 aa, synthetic construct
HSSKLQEDA

SEQ ID NO: 77 - protein, 7 aa, synthetic construct
HSSKLQL

SEQ ID NO: 78 - protein, 8 aa, synthetic construct
HTGRSGAL

SEQ ID NO: 79 - protein, 4 aa, synthetic construct
IDGR

SEQ ID NO: 80 - protein, 4 aa, synthetic construct
IEGR

SEQ ID NO: 81 - protein, 8 aa, synthetic construct
ILPRSPAF

SEQ ID NO: 82 - protein, 8 aa, synthetic construct
IPVSLRSG

SEQ ID NO: 83 - protein, 5 aa, synthetic construct
ISSGL

SEQ ID NO: 84 - protein, 6 aa, synthetic construct
ISSGLL

SEQ ID NO: 85 - protein, 7 aa, synthetic construct
ISSGLLS

SEQ ID NO: 86 - protein, 8 aa, synthetic construct
ISSGLLSS

SEQ ID NO: 87 - protein, 7 aa, synthetic construct
ISSGLSS

SEQ ID NO: 88 - protein, 8 aa, synthetic construct
KGSGDVEG

SEQ ID NO: 89 - protein, 9 aa, synthetic construct
KQEQNPGST

SEQ ID NO: 90 - protein, 8 aa, synthetic construct
KRALGLPG

SEQ ID NO: 91 - protein, 8 aa, synthetic construct
LAAPLGLL

SEQ ID NO: 92 - protein, 9 aa, synthetic construct
LAPLGLQRR

SEQ ID NO: 93 - protein, 8 aa, synthetic construct
LAQKLKSS

SEQ ID NO: 94 - protein, 8 aa, synthetic construct
LAQRLRSS

SEQ ID NO: 95 - protein, 5 aa, synthetic construct
LEATA

SEQ ID NO: 96 - protein, 8 aa, synthetic construct
LKAAPRWA

SEQ ID NO: 97 - protein, 8 aa, synthetic construct
LLAPSHRA

SEQ ID NO: 98 - protein, 8 aa, synthetic construct
LPGGLSPW

SEQ ID NO: 99 - protein, 8 aa, synthetic construct
LSGRSANI

SEQ ID NO: 100 - protein, 8 aa, synthetic construct
LSGRSANP

SEQ ID NO: 101 - protein, 8 aa, synthetic construct
LSGRSDDH

SEQ ID NO: 102 - protein, 8 aa, synthetic construct
LSGRSDIH

SEQ ID NO: 103 - protein, 8 aa, synthetic construct
LSGRSDNH

SEQ ID NO: 104 - protein, 8 aa, synthetic construct
LSGRSDNI

SEQ ID NO: 105 - protein, 8 aa, synthetic construct
LSGRSDNP

SEQ ID NO: 106 - protein, 8 aa, synthetic construct
LSGRSDQG

SEQ ID NO: 107 - protein, 8 aa, synthetic construct
LSGRSDQH

SEQ ID NO: 108 - protein, 8 aa, synthetic construct
LSGRSDTH

SEQ ID NO: 109 - protein, 8 aa, synthetic construct
LSGRSDYH

SEQ ID NO: 110 - protein, 8 aa, synthetic construct
LSGRSGNH

SEQ ID NO: 111 - protein, 10 aa, synthetic construct
LVLASSSFGY

SEQ ID NO: 112 - protein, 8 aa, synthetic construct
MDAFLESS

SEQ ID NO: 113 - protein, 8 aa, synthetic construct
MGLFSEAG

SEQ ID NO: 114 - protein, 8 aa, synthetic construct
MIAPVAYR

SEQ ID NO: 115 - protein, 8 aa, synthetic construct
MVLGRSLL

SEQ ID NO: 116 - protein, 12 aa, synthetic construct
NTLSGRSENHSG

SEQ ID NO: 117 - protein, 12 aa, synthetic construct
NTLSGRSGNHGS

SEQ ID NO: 118 - protein, 8 aa, synthetic construct
PAGLWLDP

SEQ ID NO: 119 - protein, 8 aa, synthetic construct
PGGPAGIG

SEQ ID NO: 120 - protein, 5 aa, synthetic construct
  SITE 4: note = Ethyl phenylalanine
PICFF

SEQ ID NO: 121 - protein, 6 aa, synthetic construct
  SITE 5: note = Methyl alanine
PLGCAG

SEQ ID NO: 122 - protein, 4 aa, synthetic construct
PLGL

SEQ ID NO: 123 - protein, 6 aa, synthetic construct
PLGLAG

SEQ ID NO: 124 - protein, 6 aa, synthetic construct
  VARIANT 6: note = Any amino acid
PLGLAX

SEQ ID NO: 125 - protein, 6 aa, synthetic construct
PLGLWA

SEQ ID NO: 126 - protein, 7 aa, synthetic construct
PLGLWSQ

SEQ ID NO: 127 - protein, 8 aa, synthetic construct
PLTGRSGG

SEQ ID NO: 128 - protein, 5 aa, synthetic construct
PMAKK

SEQ ID NO: 129 - protein, 6 aa, synthetic construct
PPRSFL

SEQ ID NO: 130 - protein, 8 aa, synthetic construct
PRFRIIGG

SEQ ID NO: 131 - protein, 8 aa, synthetic construct
PVGYTSSL

SEQ ID NO: 132 - protein, 8 aa, synthetic construct
PVQPIGPQ

SEQ ID NO: 133 - protein, 8 aa, synthetic construct
QALAMSAI

SEQ ID NO: 134 - protein, 8 aa, synthetic construct
QGRAITFI

SEQ ID NO: 135 - protein, 8 aa, synthetic construct
QNQALRMA

SEQ ID NO: 136 - protein, 4 aa, synthetic construct
RGPA

SEQ ID NO: 137 - protein, 8 aa, synthetic construct
RGPAFNPM

SEQ ID NO: 138 - protein, 8 aa, synthetic construct
RGPATPIM

SEQ ID NO: 139 - protein, 14 aa, synthetic construct
RKSSIIIRMRDVVL

SEQ ID NO: 140 - protein, 7 aa, synthetic construct
RLQLKAC

SEQ ID NO: 141 - protein, 6 aa, synthetic construct
RLQLKL

SEQ ID NO: 142 - protein, 8 aa, synthetic construct
RMHLRSLG

SEQ ID NO: 143 - protein, 8 aa, synthetic construct
RPSPMWAY

SEQ ID NO: 144 - protein, 8 aa, synthetic construct
RQARVVNG

SEQ ID NO: 145 - protein, 8 aa, synthetic construct
SAGFSLPA

SEQ ID NO: 146 - protein, 8 aa, synthetic construct
SAPAVESE

SEQ ID NO: 147 - protein, 8 aa, synthetic construct
SARGPSRW

SEQ ID NO: 148 - protein, 9 aa, synthetic construct
SGEPAYYTA

SEQ ID NO: 149 - protein, 8 aa, synthetic construct
SGGPLGVR

SEQ ID NO: 150 - protein, 10 aa, synthetic construct
SGRIGFLRTA

SEQ ID NO: 151 - protein, 5 aa, synthetic construct
SGRSA

SEQ ID NO: 152 - protein, 9 aa, synthetic construct
SGRSANPRG

SEQ ID NO: 153 - protein, 8 aa, synthetic construct
SMLRSMPL

SEQ ID NO: 154 - protein, 8 aa, synthetic construct
SPLPLRVP

SEQ ID NO: 155 - protein, 8 aa, synthetic construct
SPLTGRSG

SEQ ID NO: 156 - protein, 8 aa, synthetic construct
SPRSIMLA

SEQ ID NO: 157 - protein, 8 aa, synthetic construct
SSRGPAYL

SEQ ID NO: 158 - protein, 9 aa, synthetic construct
SSRHRRALD

SEQ ID NO: 159 - protein, 15 aa, synthetic construct
SSSFDKGKYKKGDDA

SEQ ID NO: 160 - protein, 15 aa, synthetic construct
SSSFDKGKYKRGDDA

SEQ ID NO: 161 - protein, 8 aa, synthetic construct
STFPFGMF

SEQ ID NO: 162 - protein, 8 aa, synthetic construct
TARGPSFK

SEQ ID NO: 163 - protein, 8 aa, synthetic construct
TGRGPSWV

SEQ ID NO: 164 - protein, 8 aa, synthetic construct
TSGRSANP

SEQ ID NO: 165 - protein, 12 aa, synthetic construct
TSTSGRSANPRG

SEQ ID NO: 166 - protein, 8 aa, synthetic construct
VAGRSMRP

SEQ ID NO: 167 - protein, 8 aa, synthetic construct
VAQFVLTE

SEQ ID NO: 168 - protein, 10 aa, synthetic construct
VHMPLGFLGP

SEQ ID NO: 169 - protein, 8 aa, synthetic construct
VPLSLYSG

SEQ ID NO: 170 - protein, 8 aa, synthetic construct
VVPEGRRS

SEQ ID NO: 171 - protein, 8 aa, synthetic construct
WATPRPMR

SEQ ID NO: 172 - protein, 8 aa, synthetic construct
YGAGLGVV

SEQ ID NO: 173 - protein, 8 aa, synthetic construct
HPVGLLAR

SEQ ID NO: 174 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 175 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 176 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 177 - protein, 20 aa, synthetic construct
  VARIANT 1..20: note = This sequence may encompass 1-10 GS repeating units
GSGSGSGSGSGSGSGSGSGS

SEQ ID NO: 178 - protein, 50 aa, synthetic construct
  VARIANT 1..50: note = This sequence may encompass 1-10 GSGGS repeating units
GSGGSGSGGSGSGGSGSGGSGSGGSGSGGSGSGGSGSGGSGSGGSGSGGS

SEQ ID NO: 179 - protein, 10 aa, synthetic construct
GGGGSGGGGS

SEQ ID NO: 180 - protein, 15 aa, synthetic construct
GGGGSGGGGSGGGGS

SEQ ID NO: 181 - protein, 20 aa, synthetic construct
GGGGSGGGGSGGGGSGGGGS

SEQ ID NO: 182 - protein, 15 aa, synthetic construct
GGGKSGGGKSGGGKS

SEQ ID NO: 183 - protein, 15 aa, synthetic construct
GGGKSGGKGSGKGGS

SEQ ID NO: 184 - protein, 4 aa, synthetic construct
GGGS

SEQ ID NO: 185 - protein, 5 aa, synthetic construct
GGGSG

SEQ ID NO: 186 - protein, 15 aa, synthetic construct
GGKGSGGKGSGGKGS

SEQ ID NO: 187 - protein, 13 aa, synthetic construct
GGSGGGGSGGGGS

SEQ ID NO: 188 - protein, 6 aa, synthetic construct
GGSGGS

SEQ ID NO: 189 - protein, 11 aa, synthetic construct
GGSGGSGGSGS

SEQ ID NO: 190 - protein, 5 aa, synthetic construct
GSGGG

SEQ ID NO: 191 - protein, 5 aa, synthetic construct
GSGSG

SEQ ID NO: 192 - protein, 4 aa, synthetic construct
GSSG

SEQ ID NO: 193 - protein, 10 aa, synthetic construct
GSSGGSGGSG

SEQ ID NO: 194 - protein, 11 aa, synthetic construct
GSSGGSGGSGG

SEQ ID NO: 195 - protein, 12 aa, synthetic construct
GSSGGSGGSGGS

SEQ ID NO: 196 - protein, 13 aa, synthetic construct
GSSGGSGGSGGSG

SEQ ID NO: 197 - protein, 16 aa, synthetic construct
GSSGGSGGSGGSGGGS

SEQ ID NO: 198 - protein, 11 aa, synthetic construct
GSSGGSGGSGS

SEQ ID NO: 199 - protein, 5 aa, synthetic construct
GSSGT

SEQ ID NO: 200 - protein, 5 aa, synthetic construct
GSSSG

SEQ ID NO: 201 - protein, 6 aa, synthetic construct
QGQSGQ

SEQ ID NO: 202 - protein, 7 aa, synthetic construct
QGQSGQG

SEQ ID NO: 203 - protein, 6 aa, synthetic construct
QGQSGS

SEQ ID NO: 204 - protein, 4 aa, synthetic construct
QSGQ

SEQ ID NO: 205 - protein, 5 aa, synthetic construct
QSGQG

SEQ ID NO: 206 - protein, 4 aa, synthetic construct
QSGS

SEQ ID NO: 207 - protein, 4 aa, synthetic construct
SGQG

SEQ ID NO: 208 - protein, 10 aa, synthetic construct
  VARIANT 1..10: note = This sequence may encompass 1-10 residues
GGGGGGGGGG

SEQ ID NO: 209 - protein, 35 aa, synthetic construct
GGGISSGLLSGRSDNHGGGISSGLLSGRSDNHGGS

SEQ ID NO: 210 - protein, 35 aa, synthetic construct
GGSGGSIPVSLRSGGGISSGLLSGRSDNHGGSGGS

SEQ ID NO: 211 - protein, 35 aa, synthetic construct
GGSGGSVPLSLYSGGGISSGLLSGRSDNHGGSGGS

SEQ ID NO: 212 - protein, 35 aa, synthetic construct
GGSHPVGLLARGGGHPVGLLARGGGHPVGLLARGS

SEQ ID NO: 213 - protein, 36 aa, synthetic construct
GGSHPVGLLARGGGHPVGLLARGGSGRSAGGSGRSA

SEQ ID NO: 214 - protein, 18 aa, synthetic construct
AVGLLAPPGGLSGRSANI

SEQ ID NO: 215 - protein, 18 aa, synthetic construct
AVGLLAPPGGLSGRSANP

SEQ ID NO: 216 - protein, 18 aa, synthetic construct
AVGLLAPPGGLSGRSDDH

SEQ ID NO: 217 - protein, 18 aa, synthetic construct
AVGLLAPPGGLSGRSDIH

SEQ ID NO: 218 - protein, 18 aa, synthetic construct
AVGLLAPPGGLSGRSDNH

SEQ ID NO: 219 - protein, 18 aa, synthetic construct
AVGLLAPPGGLSGRSDNI

SEQ ID NO: 220 - protein, 18 aa, synthetic construct
AVGLLAPPGGLSGRSDNP

SEQ ID NO: 221 - protein, 18 aa, synthetic construct
AVGLLAPPGGLSGRSDQH

SEQ ID NO: 222 - protein, 18 aa, synthetic construct
AVGLLAPPGGLSGRSDTH

SEQ ID NO: 223 - protein, 18 aa, synthetic construct
AVGLLAPPGGLSGRSDYH

SEQ ID NO: 224 - protein, 22 aa, synthetic construct
AVGLLAPPGGTSTSGRSANPRG

SEQ ID NO: 225 - protein, 17 aa, synthetic construct
AVGLLAPPSGRSANPRG

SEQ ID NO: 226 - protein, 18 aa, synthetic construct
AVGLLAPPTSGRSANPRG

SEQ ID NO: 227 - protein, 30 aa, synthetic construct
GGALFKSSFPGPAGLYAQPLAQKLKSSGGK

SEQ ID NO: 228 - protein, 33 aa, synthetic construct
GGGGSGGGGSGGGGSFVGGTGGGGSGGGGSGGS

SEQ ID NO: 229 - protein, 34 aa, synthetic construct
GGGGSGGGGSGGGGSISSGLLSGRSDNHGGSGGS

SEQ ID NO: 230 - protein, 34 aa, synthetic construct
GGGGSGGGGSGGGGSVPLSLYSGGGSGGSGGSGS

SEQ ID NO: 231 - protein, 35 aa, synthetic construct
GGGGSGGGGSGPLGLWSQGGGGSGGGGSGGGGSGG

SEQ ID NO: 232 - protein, 33 aa, synthetic construct
GGGGSGGGGSKKAAPGGGGSGGGGSGGGGSGGS

SEQ ID NO: 233 - protein, 33 aa, synthetic construct
GGGGSGGGGSKKAAPVNGGGGGSGGGGSGGGGS

SEQ ID NO: 234 - protein, 33 aa, synthetic construct
GGGGSGGGGSPMAKKGGGGSGGGGSGGGGSGGS

SEQ ID NO: 235 - protein, 33 aa, synthetic construct
GGGGSGGGGSPMAKKVNGGGGGSGGGGSGGGGS

SEQ ID NO: 236 - protein, 33 aa, synthetic construct
GGGGSGGGGSQARAKGGGGSGGGGSGGGGSGGS

SEQ ID NO: 237 - protein, 33 aa, synthetic construct
GGGGSGGGGSQARAKVNGGGGGSGGGGSGGGGS

SEQ ID NO: 238 - protein, 33 aa, synthetic construct
GGGGSGGGGSRQARVVNGGGGGSGGGGSGGGGS

SEQ ID NO: 239 - protein, 41 aa, synthetic construct
GGGGSGGGGSRQARVVNGGGGGSVPLSLYSGGGGGSGGGGS

SEQ ID NO: 240 - protein, 36 aa, synthetic construct
GGGGSGGGGSRQARVVNSVPLSLYSGGGGGSGGGGS

SEQ ID NO: 241 - protein, 33 aa, synthetic construct
GGGGSGGGGSVHMPLGFLGPGGGGSGGGGSGGS

SEQ ID NO: 242 - protein, 28 aa, synthetic construct
GGGGSVHMPLGFLGPGRSRGSFPGGGGS

SEQ ID NO: 243 - protein, 33 aa, synthetic construct
GGGGSVHMPLGFLGPPMAKKGGGGSGGGGSGGS

SEQ ID NO: 244 - protein, 32 aa, synthetic construct
GGGGSVHMPLGFLGPRQARVVNGGGGSGGGGS

SEQ ID NO: 245 - protein, 34 aa, synthetic construct
GGGGSVHMPLGFLGPRQARVVNGGGGSGGGGSGG

SEQ ID NO: 246 - protein, 30 aa, synthetic construct
GGPLAQKLKSSALFKSSFPGPAGLYAQGGR

SEQ ID NO: 247 - protein, 19 aa, synthetic construct
GLSGRSDNHGGAVGLLAPP

SEQ ID NO: 248 - protein, 21 aa, synthetic construct
GLSGRSDNHGGVHMPLGFLGP

SEQ ID NO: 249 - protein, 13 aa, synthetic construct
ISSGLLSGRSANI

SEQ ID NO: 250 - protein, 13 aa, synthetic construct
ISSGLLSGRSANP

SEQ ID NO: 251 - protein, 15 aa, synthetic construct
ISSGLLSGRSANPRG

SEQ ID NO: 252 - protein, 13 aa, synthetic construct
ISSGLLSGRSDDH

SEQ ID NO: 253 - protein, 13 aa, synthetic construct
ISSGLLSGRSDIH

SEQ ID NO: 254 - protein, 13 aa, synthetic construct
ISSGLLSGRSDNH

SEQ ID NO: 255 - protein, 13 aa, synthetic construct
ISSGLLSGRSDNI

SEQ ID NO: 256 - protein, 13 aa, synthetic construct
ISSGLLSGRSDNP

SEQ ID NO: 257 - protein, 13 aa, synthetic construct
ISSGLLSGRSDQH

SEQ ID NO: 258 - protein, 13 aa, synthetic construct
ISSGLLSGRSDTH

SEQ ID NO: 259 - protein, 13 aa, synthetic construct
ISSGLLSGRSDYH

SEQ ID NO: 260 - protein, 13 aa, synthetic construct
ISSGLLSGRSGNH

SEQ ID NO: 261 - protein, 22 aa, synthetic construct
ISSGLLSSGGSGGSLSGRSDNH

SEQ ID NO: 262 - protein, 22 aa, synthetic construct
ISSGLLSSGGSGGSLSGRSGNH

SEQ ID NO: 263 - protein, 30 aa, synthetic construct
KGGPGGPAGIGPLAQRLRSSALFKSSFPGR

SEQ ID NO: 264 - protein, 30 aa, synthetic construct
KSGPGGPAGIGALFFSSPPLAQKLKSSGGR

SEQ ID NO: 265 - protein, 18 aa, synthetic construct
LSGRSDNHGGAVGLLAPP

SEQ ID NO: 266 - protein, 22 aa, synthetic construct
LSGRSDNHGGSGGSISSGLLSS

SEQ ID NO: 267 - protein, 22 aa, synthetic construct
LSGRSDNHGGSGGSQNQALRMA

SEQ ID NO: 268 - protein, 20 aa, synthetic construct
LSGRSDNHGGVHMPLGFLGP

SEQ ID NO: 269 - protein, 22 aa, synthetic construct
LSGRSGNHGGSGGSISSGLLSS

SEQ ID NO: 270 - protein, 22 aa, synthetic construct
LSGRSGNHGGSGGSQNQALRMA

SEQ ID NO: 271 - protein, 22 aa, synthetic construct
QNQALRMAGGSGGSLSGRSDNH

SEQ ID NO: 272 - protein, 22 aa, synthetic construct
QNQALRMAGGSGGSLSGRSGNH

SEQ ID NO: 273 - protein, 30 aa, synthetic construct
RGGALFKSSFPLAQKLKSSGPAGLYAQGGK

SEQ ID NO: 274 - protein, 30 aa, synthetic construct
RGGGPAGLYAQPLAQKLKSSALFKSSFPGG

SEQ ID NO: 275 - protein, 30 aa, synthetic construct
SGGFPRSGGSFNPRTFGSKRKRRGSRGGGG

SEQ ID NO: 276 - protein, 30 aa, synthetic construct
SGPLAQKLKSSGPAGLYAQALFKSSFPGSK

SEQ ID NO: 277 - protein, 22 aa, synthetic construct
TSTSGRSANPRGGGAVGLLAPP

SEQ ID NO: 278 - protein, 24 aa, synthetic construct
TSTSGRSANPRGGGVHMPLGFLGP

SEQ ID NO: 279 - protein, 20 aa, synthetic construct
VHMPLGFLGPGGLSGRSDNH

SEQ ID NO: 280 - protein, 24 aa, synthetic construct
VHMPLGFLGPGGTSTSGRSANPRG

SEQ ID NO: 281 - protein, 21 aa, synthetic construct
SGRSAGGGSGRSAGGGSGRSA

SEQ ID NO: 282 - protein, 35 aa, synthetic construct
HPVGLLARGGGHPVGLLARGGGSGRSAGGGSGRSA

SEQ ID NO: 283 - protein, 8 aa, synthetic construct
GPLGVRGK

SEQ ID NO: 284 - protein, 8 aa, synthetic construct
VPMSMRGG

SEQ ID NO: 285 - protein, 8 aa, synthetic construct
RPFSMIMG

SEQ ID NO: 286 - protein, 8 aa, synthetic construct
VPLSLTMG

SEQ ID NO: 287 - protein, 8 aa, synthetic construct
IPESLRAG

SEQ ID NO: 288 - protein, 23 aa, synthetic construct
VPLSLYSGGGISSGLLSGRSDNH

SEQ ID NO: 289 - protein, 20 aa, synthetic construct
GGGISSGLLSGRSDNHGGGS

SEQ ID NO: 290 - protein, 15 aa, synthetic construct
GGGHPVGLLARGGGS

SEQ ID NO: 291 - protein, 36 aa, synthetic construct
GGGSGGGSGGGGISSGLLSGRSDNHGGGSGGGSGGS

SEQ ID NO: 292 - protein, 36 aa, synthetic construct
GGGGISSGLLSGRSDNHGGGISSGLLSGRSDNHGGS

SEQ ID NO: 293 - protein, 36 aa, synthetic construct
GGGSGGSIPVSLRSGGGISSGLLSGRSDNHGGSGGS

SEQ ID NO: 294 - protein, 36 aa, synthetic construct
GGGSGGSVPLSLYSGGGISSGLLSGRSDNHGGSGGS

SEQ ID NO: 295 - protein, 36 aa, synthetic construct
GGGSHPVGLLARGGGHPVGLLARGGGHPVGLLARGS

SEQ ID NO: 296 - protein, 36 aa, synthetic construct
GGGSHPVGLLARGGGHPVGLLARGGSGRSAGGSGRS

SEQ ID NO: 297 - protein, 15 aa, synthetic construct
GISSGLLSGRSDNHG

SEQ ID NO: 298 - protein, 21 aa, synthetic construct
GGGSISSGLLSGRSDNHGGGS

SEQ ID NO: 299 - protein, 11 aa, synthetic construct
  VARIANT 1..10: note = This region may encompass 1-10 residues
GGGGGGGGGGS

SEQ ID NO: 300 - protein, 11 aa, synthetic construct
  VARIANT 2..11: note = This region may encompass 1-10 residues
SGGGGGGGGGG

SEQ ID NO: 301 - protein, 5 aa, synthetic construct
GGGGS

SEQ ID NO: 302 - protein, 4 aa, synthetic construct
GGGG

SEQ ID NO: 303 - protein, 5 aa, synthetic construct
GGGGG

SEQ ID NO: 304 - protein, 6 aa, synthetic construct
GGGGGG

SEQ ID NO: 305 - protein, 7 aa, synthetic construct
GGGGGGG

SEQ ID NO: 306 - protein, 8 aa, synthetic construct
GGGGGGGG

SEQ ID NO: 307 - protein, 9 aa, synthetic construct
GGGGGGGGG

SEQ ID NO: 308 - protein, 35 aa, synthetic construct
GGGGSLALGPGGGGGSLALGPGGGGGSLALGPGGS

SEQ ID NO: 309 - protein, 25 aa, synthetic construct
GGGGSGGGGSGGGGSGGGGSGGGGS

SEQ ID NO: 310 - protein, 5 aa, synthetic construct
ADAAP

SEQ ID NO: 311 - protein, 11 aa, synthetic construct
ADAAPTVSIFP

SEQ ID NO: 312 - protein, 12 aa, synthetic construct
ADAAPTVSIFPP

SEQ ID NO: 313 - protein, 6 aa, synthetic construct
AKTTAP

SEQ ID NO: 314 - protein, 13 aa, synthetic construct
AKTTAPSVYPLAP

SEQ ID NO: 315 - protein, 17 aa, synthetic construct
AKTTPKLEEGEFSEARV

SEQ ID NO: 316 - protein, 9 aa, synthetic construct
AKTTPKLGG

SEQ ID NO: 317 - protein, 6 aa, synthetic construct
AKTTPP

SEQ ID NO: 318 - protein, 13 aa, synthetic construct
AKTTPPSVTPLAP

SEQ ID NO: 319 - protein, 6 aa, synthetic construct
ASTKGP

SEQ ID NO: 320 - protein, 26 aa, synthetic construct
ASTKGPSVFPLAPASTKGPSVFPLAP

SEQ ID NO: 321 - protein, 14 aa, synthetic construct
EGKSSGSGSESKST

SEQ ID NO: 322 - protein, 15 aa, synthetic construct
GEGESGEGESGEGES

SEQ ID NO: 323 - protein, 20 aa, synthetic construct
GEGESGEGESGEGESGEGES

SEQ ID NO: 324 - protein, 15 aa, synthetic construct
GEGGSGEGGSGEGGS

SEQ ID NO: 325 - protein, 15 aa, synthetic construct
GENKVEYAPALMALS

SEQ ID NO: 326 - protein, 15 aa, synthetic construct
GGEGSGGEGSGGEGS

SEQ ID NO: 327 - protein, 15 aa, synthetic construct
GGGESGGEGSGEGGS

SEQ ID NO: 328 - protein, 15 aa, synthetic construct
GGGESGGGESGGGES

SEQ ID NO: 329 - protein, 4 aa, synthetic construct
GGSG

SEQ ID NO: 330 - protein, 5 aa, synthetic construct
GGSGG

SEQ ID NO: 331 - protein, 9 aa, synthetic construct
GGSGGGGSG

SEQ ID NO: 332 - protein, 15 aa, synthetic construct
GHEAAAVMQVQYPAS

SEQ ID NO: 333 - protein, 15 aa, synthetic construct
GKGGSGKGGSGKGGS

SEQ ID NO: 334 - protein, 15 aa, synthetic construct
GKGKSGKGKSGKGKS

SEQ ID NO: 335 - protein, 20 aa, synthetic construct
GKGKSGKGKSGKGKSGKGKS

SEQ ID NO: 336 - protein, 15 aa, synthetic construct
GKPGSGKPGSGKPGS

SEQ ID NO: 337 - protein, 21 aa, synthetic construct
GKPGSGKPGSGKPGSGKPSGS

SEQ ID NO: 338 - protein, 15 aa, synthetic construct
GPAKELTPLKEAKVS

SEQ ID NO: 339 - protein, 12 aa, synthetic construct
GSAGSAAGSGEF

SEQ ID NO: 340 - protein, 18 aa, synthetic construct
GSTSGSGKPGSGEGSTKG

SEQ ID NO: 341 - protein, 19 aa, synthetic construct
GTAAAGAGAAGGAAAGAAG

SEQ ID NO: 342 - protein, 19 aa, synthetic construct
GTSGSSGSGSGGSGSGGGG

SEQ ID NO: 343 - protein, 14 aa, synthetic construct
IRPRAIGGSKPRVA

SEQ ID NO: 344 - protein, 18 aa, synthetic construct
KESGSVSSEQLAQFRSLD

SEQ ID NO: 345 - protein, 15 aa, synthetic construct
KTTPKLEEGEFSEAR

SEQ ID NO: 346 - protein, 19 aa, synthetic construct
PRGASKSGSASQTGSAPGS

SEQ ID NO: 347 - protein, 6 aa, synthetic construct
QPKAAP

SEQ ID NO: 348 - protein, 13 aa, synthetic construct
QPKAAPSVTLFPP

SEQ ID NO: 349 - protein, 27 aa, synthetic construct
RADAAAAGGGGSGGGGSGGGGSGGGGS

SEQ ID NO: 350 - protein, 12 aa, synthetic construct
RADAAAAGGPGS

SEQ ID NO: 351 - protein, 6 aa, synthetic construct
RADAAP

SEQ ID NO: 352 - protein, 9 aa, synthetic construct
RADAAPTVS

SEQ ID NO: 353 - protein, 6 aa, synthetic construct
SAKTTP

SEQ ID NO: 354 - protein, 18 aa, synthetic construct
SAKTTPKLEEGEFSEARV

SEQ ID NO: 355 - protein, 10 aa, synthetic construct
SAKTTPKLGG

SEQ ID NO: 356 - protein, 14 aa, synthetic construct
STAGDTHLGGEDFD

SEQ ID NO: 357 - protein, 5 aa, synthetic construct
TVAAP

SEQ ID NO: 358 - protein, 12 aa, synthetic construct
TVAAPSVFIFPP

SEQ ID NO: 359 - protein, 24 aa, synthetic construct
TVAAPSVFIFPPTVAAPSVFIFPP

SEQ ID NO: 360 - protein, 46 aa, synthetic construct
AGSGNSSGSGGSGGSGNSSGSGGSPVPSTPPTPSPSTPPTPSPSAS

SEQ ID NO: 361 - protein, 7 aa, synthetic construct
GGGGSAS

SEQ ID NO: 362 - protein, 12 aa, synthetic construct
GGGGSGGGGSAS

SEQ ID NO: 363 - protein, 17 aa, synthetic construct
GGGGSGGGGSGGGGSAS

SEQ ID NO: 364 - protein, 27 aa, synthetic construct
GGGGSGGGGSGGGGSGGGGSGGGGSAS

SEQ ID NO: 365 - protein, 32 aa, synthetic construct
GGGGSGGGGSGGGGSGGGGSGGGGSGGGGSAS

SEQ ID NO: 366 - protein, 37 aa, synthetic construct
AGGGSGGGGSGGGGSGGGGSGGGGSGGGGSGGGGSAS

SEQ ID NO: 367 - protein, 42 aa, synthetic construct
AGGGSGGGGSGGGGSGGGGSGGGGSGGGGSGGGGSGGGGSAS

SEQ ID NO: 368 - protein, 47 aa, synthetic construct
AGGGSGGGGSGGGGSGGGGSGGGGSGGGGSGGGGSGGGGSGGGGSAS

SEQ ID NO: 369 - protein, 231 aa, unidentified
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 370 - protein, 235 aa, unidentified
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 329 aa, unidentified
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 326 aa, unidentified
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 373 - protein, 329 aa, unidentified
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 374 - protein, 326 aa, unidentified
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 375 - protein, 4 aa, synthetic construct
CPPC

SEQ ID NO: 376 - protein, 4 aa, Homo sapiens
CPSC

SEQ ID NO: 377 - protein, 17 aa, unidentified
ESKYGPPCPPCPAPPVA

SEQ ID NO: 378 - protein, 11 aa, unidentified
ESKYGPPCPPC

SEQ ID NO: 379 - protein, 17 aa, unidentified
ESKYGPPCPPCPAPPCA

SEQ ID NO: 380 - protein, 20 aa, unidentified
EPKSCDKTHTCPPCPAPPVA

SEQ ID NO: 381 - protein, 15 aa, synthetic construct
CPPCPAPGGGGPSVF

SEQ ID NO: 382 - protein, 14 aa, synthetic construct
CPPCPAPGGGPSVF

SEQ ID NO: 383 - protein, 13 aa, synthetic construct
CPPCPAPGGPSVF

SEQ ID NO: 384 - protein, 12 aa, synthetic construct
CPPCPAPGPSVF

SEQ ID NO: 385 - protein, 866 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKISSGLLSGRSDNHCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEISSGLLSGRSDNHESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 386 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 387 - protein, 638 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYISSGLLSGRSDNHCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEISSGLLSGRSDNHGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 388 - protein, 626 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYISSGLLSGRSDNHTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQISSGLLSGRSDNHGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 389 - protein, 631 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYISSGLLSGRSDNHCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQISSGLLSGRSDNHGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 390 - protein, 655 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAISSGLLSGRSDNHCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEISSGLLSGRSDNHESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 391 - protein, 642 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCISSGLLSGRSDNHCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEISSGLLSGRSDNHCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 392 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 394 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 395 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 396 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK